SEQUENCE LISTING (37 sequences)

SEQ ID NO: 1 - DNA, 1424 bp, Cauliflower mosaic virus
  source 1..1424: mol_type = genomic DNA
ggaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaagtagacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgata

SEQ ID NO: 2 - DNA, 1807 bp, Escherichia coli
  source 1..1807: mol_type = genomic DNA
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtacgtatcaccgtttgtgtgaacaacgaactgaactggcagactatcccgccgggaatggtgattaccgacgaaaacggcaagaaaaagcagtcttacttccatgatttctttaactatgccgcgatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 3 - DNA, 265 bp, Agrobacterium tumefaciens
  source 1..265: mol_type = genomic DNA
gtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgac

SEQ ID NO: 4 - DNA, 1505 bp, Escherichia coli
  source 1..1505: mol_type = genomic DNA
cgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcactgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccga

SEQ ID NO: 5 - DNA, 2676 bp, Nicotiana tabacum
  source 1..2676: mol_type = genomic DNA
tgctgccggatgcaatcagtgcaaggtgcccttctcctttcccagttcaatgtctccaccccactcccacccccagtataaaaattcaggggggaaaaagagaagaaaaatatcaaaatttaaggactagttaaagttcagttttggcattttttctgcctaagtgcaatattatcaaaggtgaaaagcgcaaaaaagctctaaagtctattggggctttaagcgcaaagcgcaaataaagcatgggctttaatgcagaaaggctcaaatgaaaatgaaaagaaaaagaaaactacaaatatgtatgtatagtccaagactaatatctataagcatgaataccaaatatatggacaaagacattgaagaaactttattataaagtgaaatatcaattgtttagcatcgcctcttcaggattacgcttattggcaaggaaaagtatgcttttagagccttgatgacaacgctgaagcgcccgctaagcgaggcaaagcgctcaacatgttttgagcctcgcttcagggcttaagcgttatcaaagtgtgttctttggtgcccttgttcaagcagttctctagtatttttaccttggctccaccccactcccaccccctgtataaacgtttagggggggaaaagaaagagaagaaaaattatcaaaagttaagaaagctaagttttggcttttttctgctaggtgtgttctttggtgctcttgttcaagcagttctctagtatttttaccttggaaatgctaataagttgtcgccgtgtttccttagattgggaagcaaattaaaactatcatggaagcttcttagcattactaagtcagaaaaagcaggatatcgcttgacatacgagacaccagaaggagtagtttctcttcaaagtcgaagcattgtcatgactgtgccatcctatgtagcaagcaacatattacgtcctctttcggtgtgtttccattactgtttgctccaaagtcgttattgctattgttcatttggcactctaatatagaaacttggataaaaaaggggatcctttaatttttctttttcatttggactaggaaaagcggctcaaacacatgtataacagtcatatactgtgtataatatgtgtatgcaatataatgtgcagagaaagacttatcggagagaagagaattaaaaaaaagaacatttgtgtcacacggagaattaacatgtacatgcttaatgaggtcaagagcgcaaccgtatcccacccctcatgaaagattgaacctgtatctcatgttattgtgagcttttttcatatataaatttatgagcaattttctttaaatgctttgaccttggatttttcgaaggttactacttctgataattgctttgcaagaaatgcggctcacttaaaatgggaaacgtttatctataggttgtggttcagcttcttctgtttcttgctaaagcaagatgtctcctaaacttatattaaactcaggttgccgcagcagatgcactttcaaatttctactatcccccagttggagcagtcacaatttcatatcctcaagaagctattcgtgatgaacgtctggttgatggtgaactaaagggatttgggcagttgcatccacgtacacagggagtggaaacactaggtagctttctgtttgaaggattcactctaattttaacttcaagaatccatattgaagatggaaaacttttattacgacaattgcaagatgcaactcactgaagtagcattcctctattgcatctgcaggaataatatatagcgtatcagtcttccctgaccatcattacctagaaattttgacagtaccatgcaaatcgaagatgaatgagttcagaacaataacatcaggatttgattcatcttaaatataatacttgcgtttcatatgcaggaacgatatatagttcatcactcttccctaaccgtgccccaaaaggtcgggtgctactcttgaacatgattgcaggagcaaaaaatccggaaattttgtctaaggtaaggcatttaaattgagcgcaacttcaagtcctgccggtattgatttatcataatgaatattcatgggctggccagttggggcatcgcttgaaggttcctatggtaacaaacagaaaactgagaatgaccttatcccctgatctcttttggttgcaaatacgttaaccttgcagtgatgaggagaatgttttcagtgttattttctctagattaatggcgtagttggtttggattacaaaaccgtttaggcttatatgtgattagagtctccaggtttttgatctagcagtaagagcaaaatgtgtgatgttttaaaaaaacagtcaagtgattacaaatactcagaatagtttcaccataaagaactaaagagctaagaagattgtggtttctctttcctctatgttcgactgcattttgtgggttttccatagcatgactttcttttatggtacttgtacgaaagcatttcgctgttattctttatgcttcagggcctagaataaattgctttgagatttgatccctcattcttcttgcagacggagagccaacttgtggaagtagttgatcgtgacctcagaaaaatgcttatagaacccaaagcacaagatccccttgttgtgggtgtgcgagtatggccac

SEQ ID NO: 6 - DNA, 204 bp, Beet curly top virus
  source 1..204: mol_type = genomic DNA
tcctgtactccgatgacgtggcttagcatattaacatatctattggagtattggagtattatatatattagtacaactttcataagggccatccgttataatattaccggatggcccgaaaaaaatgggcacccaatcaaaacgtgacacgtggaaggggactgttgaatgatgtgacgtttttgagcgggaaacttcctgaag

SEQ ID NO: 7 - DNA, 1649 bp, Beet curly top virus
  source 1..1649: mol_type = genomic DNA
atgcctcctactaaaagatttcgtattcaagcaaaaaacatatttcttacatatcctcagtgttctctttcaaaagaagaagctcttgagcaaattcaaagaatacaactttcatctaataaaaaatatattaaaattgccagagagctacacgaagatgggcaacctcatctccacgtcctgcttcaactcgaaggaaaagttcagatcacaaatatcagattattcgacctggtatccccaaccaggtcagcacatttccatccaaacattcagagagctaaatccagctccgacgtcaagtcctacgtagacaaggacggagacacaattgaatggggagaattccagatcgacggtagaagtgctagaggaggtcaacagacagctaacgactcatatgccaaggcgttaaacgcaacttctcttgaccaagcacttcaaatattgaaggaagaacaaccaaaggattacttccttcaacatcacaatcttttgaacaatgctcaaaagatatttcagaggccacctgatccatggactccactatttcctctgtcctcattcacaaacgttcctgaggaaatgcaagaatgggctgatgcatatttcggggttgatgccgctgcgcggcctttaagatataatagtatcatagtagagggtgattcaagaacagggaagactatgtgggctagatctttaggggcccacaattacatcacagggcacttagattttagccctagaacgtattatgatgaagtggaatacaacgtcattgatgacgtagatcccacttacttaaagatgaaacactggaaacaccttattggagcacaaaaggagtggcagacaaacttaaagtatggaaaaccacgtgtcattaaaggtggtatcccctgcattatattatgcaatccaggacctgagagctcataccaacaatttcttgaaaaaccagaaaatgaagcccttaagtcctggacattacataattcaaccttctgcaaactccaaggtccgctctttaataaccaagcagcagcatcctcgcaaggtgactctaccctgtaactgccacttcacaatacaccatgaatgtaatagaggattttcgcacaggggaacctattactctccatcaggcaacaaattccgtcgaattcgagaatgtaccgaatccactgtatatgaaactcctatggttcgagagatacgggccaatctatcaactgaagatacaaatcagattcaactacaacctccggagagcgttgaatcttcacaagtgctggatagagctgacgataactggatcgaacaggatattgactggaccccgtttcttgaaggtcttgaaaaagagactagagatatacttggataatctaggtttaatttgtattaataatgtaattagaggtttaaatcatgtcctgtatgaagaatttacttttgtatcaagtgtaattcagaaccagagtgttgcaatgaacttgtactaatttcattattaataataaatattattaataaaaatagcatctacaattgccaaataatgtggcatacatattagtattatccgtattatcattaacaacaacatagagtaaagcattctccttcacgtcttcatacttccc

SEQ ID NO: 8 - DNA, 4140 bp, Streptococcus pyogenes
  source 1..4140: mol_type = genomic DNA
atggacaagaagtacagcatcggcctggacatcggcacgaactcggtgggctgggcggtgatcacggacgagtacaaggtgccctccaagaagttcaaggtgctgggcaacaccgaccgccactcgatcaagaagaacctgatcggcgccctgctgttcgactccggcgagaccgccgaggcgacgcgcctgaagcgcaccgcgcgtcgccgctacacgcgtcgcaagaaccgcatctgctacctgcaggagatcttcagcaacgagatggccaaggtggacgactcgttcttccaccgcctggaggagtccttcctggtggaggaagacaagaagcacgagcgccaccccatcttcggcaacatcgtggacgaggtggcctaccacgagaagtacccgacgatctaccacctgcgcaagaagctggtggacagcaccgacaaggcggacctgcgcctgatctacctggccctggcgcacatgatcaagttccgcggccacttcctgatcgagggcgacctgaaccccgacaactcggacgtggacaagctgttcatccagctggtgcagacctacaaccagctgttcgaggagaacccgatcaacgcctccggcgtggacgccaaggcgatcctgagcgcgcgcctgtccaagagccgtcgcctggagaacctgatcgcccagctgcccggcgagaagaagaacggcctgttcggcaacctgatcgcgctgtcgctgggcctgacgccgaacttcaagtccaacttcgacctggccgaggacgcgaagctgcagctgagcaaggacacctacgacgacgacctggacaacctgctggcccagatcggcgaccagtacgcggacctgttcctggccgcgaagaacctgtcggacgccatcctgctgtccgacatcctgcgcgtgaacaccgagatcacgaaggcccccctgtcggcgtccatgatcaagcgctacgacgagcaccaccaggacctgaccctgctgaaggcgctggtgcgccagcagctgccggagaagtacaaggagatcttcttcgaccagagcaagaacggctacgccggctacatcgacggcggcgcgtcgcaagaggagttctacaagttcatcaagcccatcctggagaagatggacggcacggaggagctgctggtgaagctgaaccgcgaggacctgctgcgcaagcagcgcaccttcgacaacggcagcatcccccaccagatccacctgggcgagctgcacgccatcctgcgtcgccaagaggacttctacccgttcctgaaggacaaccgcgagaagatcgagaagatcctgacgttccgcatcccctactacgtgggcccgctggcccgcggcaacagccgcttcgcgtggatgacccgcaagtcggaggagaccatcacgccctggaacttcgaggaagtggtggacaagggcgccagcgcgcagtcgttcatcgagcgcatgaccaacttcgacaagaacctgcccaacgagaaggtgctgccgaagcactccctgctgtacgagtacttcaccgtgtacaacgagctgacgaaggtgaagtacgtgaccgagggcatgcgcaagcccgccttcctgagcggcgagcagaagaaggcgatcgtggacctgctgttcaagaccaaccgcaaggtgacggtgaagcagctgaaagaggactacttcaagaagatcgagtgcttcgacagcgtggagatctcgggcgtggaggaccgcttcaacgccagcctgggcacctaccacgacctgctgaagatcatcaaggacaaggacttcctggacaacgaggagaacgaggacatcctggaggacatcgtgctgaccctgacgctgttcgaggaccgcgagatgatcgaggagcgcctgaagacgtacgcccacctgttcgacgacaaggtgatgaagcagctgaagcgtcgccgctacaccggctggggccgcctgagccgcaagctgatcaacggcatccgcgacaagcagtccggcaagaccatcctggacttcctgaagagcgacggcttcgcgaaccgcaacttcatgcagctgatccacgacgactcgctgaccttcaaagaggacatccagaaggcccaggtgtcgggccagggcgactccctgcacgagcacatcgccaacctggcgggctcccccgcgatcaagaagggcatcctgcagaccgtgaaggtggtggacgagctggtgaaggtgatgggccgccacaagccggagaacatcgtgatcgagatggcccgcgagaaccagaccacgcagaagggccagaagaacagccgcgagcgcatgaagcgcatcgaggaaggcatcaaggagctgggctcgcagatcctgaaggagcaccccgtggagaacacccagctgcagaacgagaagctgtacctgtactacctgcagaacggccgcgacatgtacgtggaccaggagctggacatcaaccgcctgtccgactacgacgtggaccacatcgtgccccagagcttcctgaaggacgactcgatcgacaacaaggtgctgacccgcagcgacaagaaccgcggcaagagcgacaacgtgccgtcggaggaagtggtgaagaagatgaagaactactggcgccagctgctgaacgccaagctgatcacgcagcgcaagttcgacaacctgaccaaggccgagcgcggtggcctgtcggagctggacaaggcgggcttcatcaagcgccagctggtggagacccgccagatcacgaagcacgtggcgcagatcctggactcccgcatgaacacgaagtacgacgagaacgacaagctgatccgcgaggtgaaggtgatcaccctgaagtccaagctggtcagcgacttccgcaaggacttccagttctacaaggtgcgcgagatcaacaactaccaccacgcccacgacgcgtacctgaacgccgtggtgggcaccgcgctgatcaagaagtaccccaagctggagagcgagttcgtgtacggcgactacaaggtgtacgacgtgcgcaagatgatcgccaagtcggagcaggagatcggcaaggccaccgcgaagtacttcttctactccaacatcatgaacttcttcaagaccgagatcacgctggccaacggcgagatccgcaagcgcccgctgatcgagaccaacggcgagacgggcgagatcgtgtgggacaagggccgcgacttcgcgaccgtgcgcaaggtgctgagcatgccccaggtgaacatcgtgaagaagaccgaggtgcagacgggcggcttctccaaggagagcatcctgccgaagcgcaactcggacaagctgatcgcccgcaagaaggactgggaccccaagaagtacggcggcttcgactccccgaccgtggcctacagcgtgctggtggtggcgaaggtggagaagggcaagtccaagaagctgaagagcgtgaaggagctgctgggcatcaccatcatggagcgcagctcgttcgagaagaaccccatcgacttcctggaggccaagggctacaaagaggtgaagaaggacctgatcatcaagctgccgaagtactcgctgttcgagctggagaacggccgcaagcgcatgctggcctccgcgggcgagctgcagaagggcaacgagctggccctgcccagcaagtacgtgaacttcctgtacctggcgtcccactacgagaagctgaagggctcgccggaggacaacgagcagaagcagctgttcgtggagcagcacaagcactacctggacgagatcatcgagcagatctcggagttctccaagcgcgtgatcctggccgacgcgaacctggacaaggtgctgagcgcctacaacaagcaccgcgacaagcccatccgcgagcaggcggagaacatcatccacctgttcaccctgacgaacctgggcgccccggccgcgttcaagtacttcgacaccacgatcgaccgcaagcgctacacctccacgaaagaggtgctggacgcgaccctgatccaccagagcatcaccggcctgtacgagacgcgcatcgacctgagccagctgggcggcgactcccgcgcggacccgaagaagaagcgcaaggtgtaa

SEQ ID NO: 9 - DNA, 201 bp, Arabidopsis thaliana
  source 1..201: mol_type = genomic DNA
catcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagt

SEQ ID NO: 10 - DNA, 166 bp, synthetic construct
  misc_feature 1..166: note = sgRNA2.0 with MS2 aptamer hairpins
  source 1..166: mol_type = other DNA
cagttcgttgttcacacaagttttagagctaggccaacatgaggatcacccatgtctgcagggcctagcaagttaaaataaggctagtccgttatcaacttggccaacatgaggatcacccatgtctgcagggccaagtggcaccgagtcggtgcttttttttttt

SEQ ID NO: 11 - DNA, 164 bp, synthetic construct
  misc_feature 1..164: note = sgRNA2.0-1 for tobacco PPOX1 gene
  source 1..164: mol_type = other DNA
attactaagtcagaaaagttttagagctaggccaacatgaggatcacccatgtctgcagggcctagcaagttaaaataaggctagtccgttatcaacttggccaacatgaggatcacccatgtctgcagggccaagtggcaccgagtcggtgcttttttttttt

SEQ ID NO: 12 - DNA, 167 bp, synthetic construct
  misc_feature 1..167: note = sgRNA2.0-2 for tobacco PPOX1 gene
  source 1..167: mol_type = other DNA
tgctactcttgaactacatggttttagagctaggccaacatgaggatcacccatgtctgcagggcctagcaagttaaaataaggctagtccgttatcaacttggccaacatgaggatcacccatgtctgcagggccaagtggcaccgagtcggtgcttttttttttt

SEQ ID NO: 13 - DNA, 406 bp, Agrobacterium tumefaciens
  source 1..406: mol_type = genomic DNA
gaattaacagaggtggatggacagacccgttcttacaccggactgggcgcgggataggatattcagattgggatgggattgagcttaaagccggcgctgagaccatgctcaaggtaggcaatgtcctcagcgtcgagcccggcatctatgtcgagggcattggtggagcgcgcttcggggataccgtgcttgtaactgagaccggatatgaggccctcactccgcttgatcttggcaaagatatttgacgcatttattagtatgtgttaattttcatttgcagtgcagtattttctattcgatctttatgtaattcgttacaattaataaatattcaaatcagattattgactgtcatttgtatcaaatcgtgtttaatggatatttttattataatattgatgat

SEQ ID NO: 14 - DNA, 6024 bp, synthetic construct
  misc_feature 1..6024: note = cas9-B-rep fusion gene
  source 1..6024: mol_type = other DNA
atggacaagaagtacagcatcggcctggacatcggcacgaactcggtgggctgggcggtgatcacggacgagtacaaggtgccctccaagaagttcaaggtgctgggcaacaccgaccgccactcgatcaagaagaacctgatcggcgccctgctgttcgactccggcgagaccgccgaggcgacgcgcctgaagcgcaccgcgcgtcgccgctacacgcgtcgcaagaaccgcatctgctacctgcaggagatcttcagcaacgagatggccaaggtggacgactcgttcttccaccgcctggaggagtccttcctggtggaggaagacaagaagcacgagcgccaccccatcttcggcaacatcgtggacgaggtggcctaccacgagaagtacccgacgatctaccacctgcgcaagaagctggtggacagcaccgacaaggcggacctgcgcctgatctacctggccctggcgcacatgatcaagttccgcggccacttcctgatcgagggcgacctgaaccccgacaactcggacgtggacaagctgttcatccagctggtgcagacctacaaccagctgttcgaggagaacccgatcaacgcctccggcgtggacgccaaggcgatcctgagcgcgcgcctgtccaagagccgtcgcctggagaacctgatcgcccagctgcccggcgagaagaagaacggcctgttcggcaacctgatcgcgctgtcgctgggcctgacgccgaacttcaagtccaacttcgacctggccgaggacgcgaagctgcagctgagcaaggacacctacgacgacgacctggacaacctgctggcccagatcggcgaccagtacgcggacctgttcctggccgcgaagaacctgtcggacgccatcctgctgtccgacatcctgcgcgtgaacaccgagatcacgaaggcccccctgtcggcgtccatgatcaagcgctacgacgagcaccaccaggacctgaccctgctgaaggcgctggtgcgccagcagctgccggagaagtacaaggagatcttcttcgaccagagcaagaacggctacgccggctacatcgacggcggcgcgtcgcaagaggagttctacaagttcatcaagcccatcctggagaagatggacggcacggaggagctgctggtgaagctgaaccgcgaggacctgctgcgcaagcagcgcaccttcgacaacggcagcatcccccaccagatccacctgggcgagctgcacgccatcctgcgtcgccaagaggacttctacccgttcctgaaggacaaccgcgagaagatcgagaagatcctgacgttccgcatcccctactacgtgggcccgctggcccgcggcaacagccgcttcgcgtggatgacccgcaagtcggaggagaccatcacgccctggaacttcgaggaagtggtggacaagggcgccagcgcgcagtcgttcatcgagcgcatgaccaacttcgacaagaacctgcccaacgagaaggtgctgccgaagcactccctgctgtacgagtacttcaccgtgtacaacgagctgacgaaggtgaagtacgtgaccgagggcatgcgcaagcccgccttcctgagcggcgagcagaagaaggcgatcgtggacctgctgttcaagaccaaccgcaaggtgacggtgaagcagctgaaagaggactacttcaagaagatcgagtgcttcgacagcgtggagatctcgggcgtggaggaccgcttcaacgccagcctgggcacctaccacgacctgctgaagatcatcaaggacaaggacttcctggacaacgaggagaacgaggacatcctggaggacatcgtgctgaccctgacgctgttcgaggaccgcgagatgatcgaggagcgcctgaagacgtacgcccacctgttcgacgacaaggtgatgaagcagctgaagcgtcgccgctacaccggctggggccgcctgagccgcaagctgatcaacggcatccgcgacaagcagtccggcaagaccatcctggacttcctgaagagcgacggcttcgcgaaccgcaacttcatgcagctgatccacgacgactcgctgaccttcaaagaggacatccagaaggcccaggtgtcgggccagggcgactccctgcacgagcacatcgccaacctggcgggctcccccgcgatcaagaagggcatcctgcagaccgtgaaggtggtggacgagctggtgaaggtgatgggccgccacaagccggagaacatcgtgatcgagatggcccgcgagaaccagaccacgcagaagggccagaagaacagccgcgagcgcatgaagcgcatcgaggaaggcatcaaggagctgggctcgcagatcctgaaggagcaccccgtggagaacacccagctgcagaacgagaagctttacctgtactacctgcagaacggccgcgacatgtacgtggaccaggagctggacatcaaccgcctgtccgactacgacgtggaccacatcgtgccccagagcttcctgaaggacgactcgatcgacaacaaggtgctgacccgcagcgacaagaaccgcggcaagagcgacaacgtgccgtcggaggaagtggtgaagaagatgaagaactactggcgccagctgctgaacgccaagctgatcacgcagcgcaagttcgacaacctgaccaaggccgagcgcggtggcctgtcggagctggacaaggcgggcttcatcaagcgccagctggtggagacccgccagatcacgaagcacgtggcgcagatcctggactcccgcatgaacacgaagtacgacgagaacgacaagctgatccgcgaggtgaaggtgatcaccctgaagtccaagctggtcagcgacttccgcaaggacttccagttctacaaggtgcgcgagatcaacaactaccaccacgcccacgacgcgtacctgaacgccgtggtgggcaccgcgctgatcaagaagtaccccaagctggagagcgagttcgtgtacggcgactacaaggtgtacgacgtgcgcaagatgatcgccaagtcggagcaggagatcggcaaggccaccgcgaagtacttcttctactccaacatcatgaacttcttcaagaccgagatcacgctggccaacggcgagatccgcaagcgcccgctgatcgagaccaacggcgagacgggcgagatcgtgtgggacaagggccgcgacttcgcgaccgtgcgcaaggtgctgagcatgccccaggtgaacatcgtgaagaagaccgaggtgcagacgggcggcttctccaaggagagcatcctgccgaagcgcaactcggacaagctgatcgcccgcaagaaggactgggaccccaagaagtacggcggcttcgactccccgaccgtggcctacagcgtgctggtggtggcgaaggtggagaagggcaagtccaagaagctgaagagcgtgaaggagctgctgggcatcaccatcatggagcgcagctcgttcgagaagaaccccatcgacttcctggaggccaagggctacaaagaggtgaagaaggacctgatcatcaagctgccgaagtactcgctgttcgagctggagaacggccgcaagcgcatgctggcctccgcgggcgagctgcagaagggcaacgagctggccctgcccagcaagtacgtgaacttcctgtacctggcgtcccactacgagaagctgaagggctcgccggaggacaacgagcagaagcagctgttcgtggagcagcacaagcactacctggacgagatcatcgagcagatctcggagttctccaagcgcgtgatcctggccgacgcgaacctggacaaggtgctgagcgcctacaacaagcaccgcgacaagcccatccgcgagcaggcggagaacatcatccacctgttcaccctgacgaacctgggcgccccggccgcgttcaagtacttcgacaccacgatcgaccgcaagcgctacacctccacgaaagaggtgctggacgcgaccctgatccaccagagcatcaccggcctgtacgagacgcgcatcgacctgagccagctgggcggcgactcccgcgcggacccgaagaagaagcgcaaggtgagcgctggaggaggtggaagcggaggaggaggaagcggaggaggaggtagcggatccatgcctcctactaaaagatttcgtattcaagcaaaaaacatatttcttacatatcctcagtgttctctttcaaaagaagaagctcttgagcaaattcaaagaatacaactttcatctaataaaaaatatattaaaattgccagagagctacacgaagatgggcaacctcatctccacgtcctgcttcaactcgaaggaaaagttcagatcacaaatatcagattattcgacctggtatccccaaccaggtaattttcatctttgtttggccttccaagtgctttttttgctgtttacgggtggaacttcagtaaaaatgggatcaaaacatcatatggcataaataaattttaagaatggcgaactcggggttaccgaatatggcttcctttttcagtgtttcttagtccattgtacttatgagattgcaggtcagcacatttccatccaaacattcagagagctaaatccagctccgacgtcaagtcctacgtagacaaggacggagacacaattgaatggggagaattccagatcgacggtagaagtgctagaggaggtcaacagacagctaacgactcatatgccaaggcgttaaacgcaacttctcttgaccaagcacttcaaatattgaaggaagaacaaccaaaggattacttccttcaacatcacaatcttttgaacaatgctcaaaagatatttcagaggccacctgatccatggactccactatttcctctgtcctcattcacaaacgttcctgaggaaatgcaagaatgggctgatgcatatttcggggttgatgccgctgcgcggcctttaagatataatagtatcatagtagagggtgattcaagaacagggaagactatgtgggctagatctttaggggcccacaattacatcacagggcacttagattttagccctagaacgtattatgatgaagtggaatacaacgtcattgatgacgtagatcccacttacttaaagatgaaacactggaaacaccttattggagcacaaaaggagtggcagacaaacttaaagtatggaaaaccacgtgtcattaaaggtggtatcccctgcattatattatgcaatccaggacctgagagcacataccaacaatttcttgaaaaaccagaaaatgaagcccttaagtcctggacattacataattcaaccttctgcaaactccaaggtccgctctttaataaccaagcagcagcatcctcgcaaggtgactctaccctgtaactgccacttcacaatacaccatgaatgtaatagaggattttcgcacaggggaacctattactctccatcaggcaacaaattccgtcgaattcgagaatgtaccgaatccactgtatatgaaactcctatggttcgagagatacgggccaatctatcaactgaagatacaaatcagattcaactacaacctccggagagcgttgaatcttcacaagtgctggatagagctgacgataactggatcgaacaggatattgactggaccccgtttcttgaaggtaagaaatcttttcccatcttgaagtcacctcaaaccgaacgttaggaaattccaaaatgttttgatagtagtctacttagtttcaagttttgggtttgtgtatactttcactaataatatgcgtggaaacattgcaggtcttgaaaaagagactagagatatacttggataatctaggtttaatttgtattaataatgtaattagaggtttaaatcatgtcctgtatgaagaatttacttttgtatcaagtgtaattcagaaccagagtgttgcaatgaacttgtactaa

SEQ ID NO: 15 - DNA, 681 bp, Bacteriophage lambda
  source 1..681: mol_type = genomic DNA
atgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatga

SEQ ID NO: 16 - DNA, 155 bp, synthetic construct
  misc_feature 1..155: note = sgRNA comprising multiple stem-loops
  source 1..155: mol_type = other DNA
gttttagagctaggccaacatgaggatcacccatgtctgcagggcctagcaagttaaaataaggctagtccgttatcaactttaaggagtttatatggaaacccttaaagtggcaccgagtcggtgctaccgccgacaacgcggttttttttttt

SEQ ID NO: 17 - DNA, 3237 bp, synthetic construct
  misc_feature 1..3237: note = Triple fusion of E.coli recA gene
  source 1..3237: mol_type = other DNA
atggacgagaacaagaagcgcgccctggccgcggccctgggacagatcgaacgccaattcggcaaaggcgcggtcatgcgcatgggcgaccatgagcgccaggcgatcccggccatctccaccggctccctgggtctggacatcgccctcggcatcggcggcctgcccaagggccggatcgtcgagatctacggtccggaatcctcgggcaagaccaccctgaccctctcggtgatcgccgaggcccagaaacagggcgccacctgtgccttcgtcgacgccgagcacgcgctcgatcccgactatgccggcaagctgggcgtcaacgtcgacgacctgctggtctcccagccggacaccggcgagcaggccctggaaatcaccgacatgctggtgcgctccaacgcggtcgacgtgatcatcgtcgactccgtggccgcgctggtacccaaggccgagatcgaaggcgagatgggcgacgcccacgtcggcctgcaggcacgcctgatgtcccaggcgctgcgcaagatcaccggcaatatcaagaacgccaactgcctggtcatcttcatcaaccagatccgcatgaagatcggcgtcatgttcggcaacccggaaaccaccaccggcggtaacgcactgaagttctacgcctcggtccgcctggacatccgtcgtaccggcgcggtgaaggaaggcgacgaggtggtgggtagcgaaacccgcgtcaaggtggtgaagaacaaggtttccccgccgttccgccaggccgagttccagatcctctacggtaagggcatctaccgtaccggcgagatcatcgatctgggcgtgcaattgggcctggtcgagaagtccggcgcctggtacagctaccagggcagcaagatcggccagggcaaggcgaacgccgccaagtacctggaagacaatccggaaatcggttcggtactggagaagaccattcgcgaccagttgctggccaagagcggcccggtgaaggccgacgccgaagaagtggctgacgccgaagccgattcagagctcggagaaggtcaaggacagggacaaggtccaggacgaggatacgcatataagcttgacgagaacaagaagcgcgccctggccgcggccctgggacagatcgaacgccaattcggcaaaggcgcggtcatgcgcatgggcgaccatgagcgccaggcgatcccggccatctccaccggctccctgggtctggacatcgccctcggcatcggcggcctgcccaagggccggatcgtcgagatctacggtccggaatcctcgggcaagaccaccctgaccctctcggtgatcgccgaggcccagaaacagggcgccacctgtgccttcgtcgacgccgagcacgcgctcgatcccgactatgccggcaagctgggcgtcaacgtcgacgacctgctggtctcccagccggacaccggcgagcaggccctggaaatcaccgacatgctggtgcgctccaacgcggtcgacgtgatcatcgtcgactccgtggccgcgctggtacccaaggccgagatcgaaggcgagatgggcgacgcccacgtcggcctgcaggcacgcctgatgtcccaggcgctgcgcaagatcaccggcaatatcaagaacgccaactgcctggtcatcttcatcaaccagatccgcatgaagatcggcgtcatgttcggcaacccggaaaccaccaccggcggtaacgcactgaagttctacgcctcggtccgcctggacatccgtcgtaccggcgcggtgaaggaaggcgacgaggtggtgggtagcgaaacccgcgtcaaggtggtgaagaacaaggtttccccgccgttccgccaggccgagttccagatcctctacggtaagggcatctaccgtaccggcgagatcatcgatctgggcgtgcaattgggcctggtcgagaagtccggcgcctggtacagctaccagggcagcaagatcggccagggcaaggcgaacgccgccaagtacctggaagacaatccggaaatcggttcggtactggagaagaccattcgcgaccagttgctggccaagagcggcccggtgaaggccgacgccgaagaagtggctgacgccgaagccgattcaggatccggagaaggtcaaggacagggacaaggtccaggacgaggatacgcatatgcatgcgacgagaacaagaagcgcgccctggccgcggccctgggacagatcgaacgccaattcggcaaaggcgcggtcatgcgcatgggcgaccatgagcgccaggcgatcccggccatctccaccggctccctgggtctggacatcgccctcggcatcggcggcctgcccaagggccggatcgtcgagatctacggtccggaatcctcgggcaagaccaccctgaccctctcggtgatcgccgaggcccagaaacagggcgccacctgtgccttcgtcgacgccgagcacgcgctcgatcccgactatgccggcaagctgggcgtcaacgtcgacgacctgctggtctcccagccggacaccggcgagcaggccctggaaatcaccgacatgctggtgcgctccaacgcggtcgacgtgatcatcgtcgactccgtggccgcgctggtacccaaggccgagatcgaaggcgagatgggcgacgcccacgtcggcctgcaggcacgcctgatgtcccaggcgctgcgcaagatcaccggcaatatcaagaacgccaactgcctggtcatcttcatcaaccagatccgcatgaagatcggcgtcatgttcggcaacccggaaaccaccaccggcggtaacgcactgaagttctacgcctcggtccgcctggacatccgtcgtaccggcgcggtgaaggaaggcgacgaggtggtgggtagcgaaacccgcgtcaaggtggtgaagaacaaggtttccccgccgttccgccaggccgagttccagatcctctacggtaagggcatctaccgtaccggcgagatcatcgatctgggcgtgcaattgggcctggtcgagaagtccggcgcctggtacagctaccagggcagcaagatcggccagggcaaggcgaacgccgccaagtacctggaagacaatccggaaatcggttcggtactggagaagaccattcgcgaccagttgctggccaagagcggcccggtgaaggccgacgccgaagaagtggctgacgccgaagccgattaa

SEQ ID NO: 18 - DNA, 3207 bp, synthetic construct
  misc_feature 1..3207: note = Triple fusion of Arabidopsis rad51 gene
  source 1..3207: mol_type = other DNA
atgacgacgatggagcagcgtagaaaccagaatgctgtccaacaacaagacgatgaagaaacccagcacggacctttccctgtcgaacagcttcaggcagcaggtattgcttctgttgatgtaaagaagcttagggatgctggtctctgtactgttgaaggtgttgcttatactccgaggaaggatctcttgcagattaaaggaattagtgatgccaaggttgacaagattgtagaagcagcttcaaagctagttcctctggggttcacaagtgcgagccagctccatgctcagagacaggaaattattcagattacctctggatcacgggagctcgataaagttctagaaggaggtattgaaactggttccatcacagagttatatggtgagttccgctctggaaagactcagctgtgccatacactgtgtgtgacttgtcaacttcccatggatcaaggaggtggagagggaaaggccatgtacattgatgctgagggaacattcaggccacaaagactcttacagatagctgacaggtttggattaaatggagctgatgtactagaaaacgttgcctatgcgagggcgtataatacagatcatcagtcaaggcttttgcttgaagcagcatcaatgatgattgaaacaaggtttgctctcctgattgtcgatagtgctaccgctctctacagaacagatttctctggaaggggagagctttcggctcgacaaatgcatcttgcaaagttcttgagaagtcttcagaagttagcagatgagtttggtgtggctgttgttataacaaaccaagtagttgcgcaagtagatggttcagctctttttgctggtccccaatttaagccgattggtgggaatatcatggctcatgccaccacaacaaggttggcgttgaggaaaggaagagcagaggagagaatctgtaaagtgataagctcgccatgtttgccagaagcggaagctcgatttcaaatatctacagaaggtgtaacagattgcaaggatgagctcggagaaggtcaaggacagggacaaggtccaggacgaggatacgcatataagcttatgacgacgatggagcagcgtagaaaccagaatgctgtccaacaacaagacgatgaagaaacccagcacggacctttccctgtcgaacagcttcaggcagcaggtattgcttctgttgatgtaaagaagcttagggatgctggtctctgtactgttgaaggtgttgcttatactccgaggaaggatctcttgcagattaaaggaattagtgatgccaaggttgacaagattgtagaagcagcttcaaagctagttcctctggggttcacaagtgcgagccagctccatgctcagagacaggaaattattcagattacctctggatcacgggagctcgataaagttctagaaggaggtattgaaactggttccatcacagagttatatggtgagttccgctctggaaagactcagctgtgccatacactgtgtgtgacttgtcaacttcccatggatcaaggaggtggagagggaaaggccatgtacattgatgctgagggaacattcaggccacaaagactcttacagatagctgacaggtttggattaaatggagctgatgtactagaaaacgttgcctatgcgagggcgtataatacagatcatcagtcaaggcttttgcttgaagcagcatcaatgatgattgaaacaaggtttgctctcctgattgtcgatagtgctaccgctctctacagaacagatttctctggaaggggagagctttcggctcgacaaatgcatcttgcaaagttcttgagaagtcttcagaagttagcagatgagtttggtgtggctgttgttataacaaaccaagtagttgcgcaagtagatggttcagctctttttgctggtccccaatttaagccgattggtgggaatatcatggctcatgccaccacaacaaggttggcgttgaggaaaggaagagcagaggagagaatctgtaaagtgataagctcgccatgtttgccagaagcggaagctcgatttcaaatatctacagaaggtgtaacagattgcaaggatggatccggagaaggtcaaggacagggacaaggtccaggacgaggatacgcatatgcatgcatgacgacgatggagcagcgtagaaaccagaatgctgtccaacaacaagacgatgaagaaacccagcacggacctttccctgtcgaacagcttcaggcagcaggtattgcttctgttgatgtaaagaagcttagggatgctggtctctgtactgttgaaggtgttgcttatactccgaggaaggatctcttgcagattaaaggaattagtgatgccaaggttgacaagattgtagaagcagcttcaaagctagttcctctggggttcacaagtgcgagccagctccatgctcagagacaggaaattattcagattacctctggatcacgggagctcgataaagttctagaaggaggtattgaaactggttccatcacagagttatatggtgagttccgctctggaaagactcagctgtgccatacactgtgtgtgacttgtcaacttcccatggatcaaggaggtggagagggaaaggccatgtacattgatgctgagggaacattcaggccacaaagactcttacagatagctgacaggtttggattaaatggagctgatgtactagaaaacgttgcctatgcgagggcgtataatacagatcatcagtcaaggcttttgcttgaagcagcatcaatgatgattgaaacaaggtttgctctcctgattgtcgatagtgctaccgctctctacagaacagatttctctggaaggggagagctttcggctcgacaaatgcatcttgcaaagttcttgagaagtcttcagaagttagcagatgagtttggtgtggctgttgttataacaaaccaagtagttgcgcaagtagatggttcagctctttttgctggtccccaatttaagccgattggtgggaatatcatggctcatgccaccacaacaaggttggcgttgaggaaaggaagagcagaggagagaatctgtaaagtgataagctcgccatgtttgccagaagcggaagctcgatttcaaatatctacagaaggtgtaacagattgcaaggattaactagtg

SEQ ID NO: 19 - DNA, 34 bp, synthetic construct
  misc_feature 1..34: note = MS2-derived stem-loop for binding
  source 1..34: mol_type = other DNA
ggccaacatgaggatcacccatgtctgcagggcc

SEQ ID NO: 20 - DNA, 25 bp, synthetic construct
  misc_feature 1..25: note = PP7-derived stem-loop for binding
  source 1..25: mol_type = other DNA
taaggagtttatatggaaaccctta

SEQ ID NO: 21 - DNA, 17 bp, synthetic construct
  misc_feature 1..17: note = P22-derived stem-loop for binding B-box
  source 1..17: mol_type = other DNA
accgccgacaacgcggt

SEQ ID NO: 22 - DNA, 385 bp, Bacteriophage MS2
  source 1..385: mol_type = genomic DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggagatcctacctgaacatggaactcactatcccaattttcgctaccaattctgactgtgaactcatcgtgaaggcaatgcaggggctcctcaaagacggcaatcctatcccttccgccatcgccgctaactcaggca

SEQ ID NO: 23 - DNA, 387 bp, Bacteriophage PP7
  source 1..387: mol_type = genomic DNA
atgtccaaaaccatcgttctttcggtcggcgaggctactcgcactctgactgagatccagtccaccgcagaccgtcagatcttcgaagagaaggtcgggcctctggtgggtcggctgcgcctcacggcttcgctccgtcaaaacggagccaagaccgcgtatcgagtcaacctaaaactggatcaggcggacgtcgttgattgctccaccagcgtctgcggcgagcttccgaaagtgcgctacactcaggtatggtcgcacgacgtgacaatcgttgcgaatagcaccgaggcctcgcgcaaatcgttgtacgatttgaccaagtccctcgtcgcgacctcgcaggtcgaagatcttgtcgtcaaccttgtgccgctgggccgttaa

SEQ ID NO: 24 - DNA, 303 bp, Bacteriophage P22
  source 1..303: mol_type = genomic DNA
atgacggttatcacctacgggaagtcaacgtttgcaggcaatgctaaaactcgccgtcatgagcggcgcagaaagctagccatagagcgcgacaccatctgcaatatcatcgattcaatttttggctgcgatgctcctgatgcttctcaggaagttaaagccaaaagaattgaccgtgtcaccaaagccatttcgcttgccggaacgcgtcagaaggaagttgaaggaggatctgtacttcttccaggcgtagcactttacgcggctggtcatcgtaagagcaaacaaataacagcgaggtaa

SEQ ID NO: 25 - DNA, 2753 bp, synthetic construct
  misc_feature 1..2753: note = MSH2 dominant-negative allele gene sequence
  source 1..2753: mol_type = other DNA
atggctgggttaaggcaggatcttagacagcatctgaagcgaatctcagatgttgagaggcttttgcgcagtctcgagagaagaagaggtgggttacagcacattattaaactctatcaggtactttccgcacttcaatctgcttctctcaatgttaacaaaattgcattttcattgtcctaaatgtgtttatgcaactctgaagttataggtatgttattaagttcattactaattaagtcttcatcttttctctgcagtcagctataaggcttcccttcatcaaaacagctatgcaacagtacaccggagaattcgcatcactcatcagcgagaggtacctgaaaaagcttgaggctttatcagatcaagatcaccttggaaagttcatcgatttggttgagtgctctgtagatcttgaccagctagaaaatggagaatacatgatatcttcaaactacgacaccaaattggcatctctgaaagatcagaaagaattgctggagcagcagattcacgaattgcacaaaaagacagcgatagaacttgatcttcaggtcgacaaggctcttaaacttgacaaagcagcgcaatttgggcatgtcttcaggatcacgaagaaggaagagccaaagatcaggaagaagctgacgacacagtttatagtgctggagactcgcaaagacggagtgaagttcacaaacacaaagctaaaaaaactgggcgaccagtaccaaagtgttgtggatgattataggagctgtcaaaaggagctcgttgatcgtgtagttgagactgttaccagcttctctgaggtatgtttagttattcatattaagcattggactgttacagaattggttgtttaaaatcatagtaaactatatgtggaatttatatgtatattgtatggttataggtatttgaggacttagctgggttactttctgaaatggatgttttgttaagctttgctgatttggctgccagttgccctactccatactgtaggccagaaatcacctctttggttagtacaatctcaagttgattattttgttctgaaaatgaatagttttttctttccaagtttatgacataatgttgagagcacggttaataaattgtaggatgctggagatattgtactagaaggaagcagacatccatgtgtagaagctcaagattgggtgaatttcataccaaatgattgcagactcgtaagtattgaatgtggtaaataaactgagacgtctttgtttttcttgtttcccttttgacttgaacaaatacttgtttgccctttactgttctttgaaatcagatgagagggaagagttggtttcaaatagtaacagggcctaacatgggagataagtccactttcatccgccaggtatgatgatttcctctagttcagttttgcttcatagacgtatgactaaagtcggtttccggccattataaatcccaggttggtgtgattgtgctgatggctcaagttggttcctttgttccttgtgataaagcatcaatttccataagagactgcatctttgcccgtgtaggagcaggcgattgccaagtgagtttaagtttagccctcaatgaacgaaaaactgctgatatcctgaacacccttattccaactttttttcctttggtgtgttagctgcgtggagtgtcaacttttatgcaagaaatgcttgaaaccgcatcgatattgaaaggcgctactgataagtcactgataattatcgatgaacttggtcgtggaacatcaacttatgatggttttggttagtttctctgcaatttctcttctttcatttggatgtttttagtaagttttctattatatattcatttttatggtcatatgtgagatttcagtgctcttgacatcatcgtggtgaatatatcaggtttagcttgggctatatgtgagcatctggttcaagtgaaaagagcaccaactctgtttgctactcacttccatgaacttactgccttggctcaagcaaactctgaggtctctggtaacactgttggtgtggcaaacttccatgtcagcgctcacattgacactgaaagccgcaaactcaccatgctttacaaggtctggtttataaattaaaaaattgctgatctgttgcagttaaaagtgtctctgtttttatgtttaatctaaattacttatttgattttcttacaaagatgaaattgaaattaattttgtgtggtgtgttgtttgtctggttaggttgaaccaggggcctgtgaccagagctttgggattcatgtggcggaatttgccaacttccctgaaagcgtcgtggccctcgcaagagagaaagctgcagagctggaagatttctctccctcctcgatgataatcaacaatgaggtcttgattcatttccccctttgtttttggttgatgatggaatcattctatcattcacccattctgcagtttatgctatattattataaatctatgtgacaaagatttaattctcgtattgttgtttgcaggagagtgggaagagaaagagcagagaagatgatccagatgaagtatcaagaggggcagagcgagctcacaagtttctgaaagagtttgcagcgatgccacttgataaaatggagcttaaagattcacttcaacgggtacgtgagatgaaagatgagctagagaaagatgctgcagactgccactggctcaggcagtttctgtgaagaacccctga

SEQ ID NO: 26 - DNA, 474 bp, Bacteriophage T4
  source 1..474: mol_type = genomic DNA
atgttattgactggcaaattatacaaagaagaaaaacagaaattttatgatgcacaaaacggtaaatgcttaatttgccaacgagaactaaatcctgatgttcaagctaatcacctcgaccatgaccatgaattaaatggaccaaaagcaggaaaggtgcgtggattgctttgtaatctatgcaatgctgcagaaggtcaaatgaagcataaatttaatcgttctggcttaaagggacaaggtgttgattatcttgaatggttagaaaatttacttacttatttaaaatccgattacacccaaaataatattcaccctaactttgttggagataaatcaaaggaattttctcgtttaggaaaagaggaaatgatggccgagatgcttcaaagaggatttgaatataatgaatctgacaccaaaacacaattaatagcttcattcaagaagcagcttagaaagagtttaaaatga

SEQ ID NO: 27 - DNA, 1062 bp, Saccharomyces cerevisiae
  source 1..1062: mol_type = genomic DNA
atgtcgacagcacagaaagctaagatattgcaactcatcgattcctgctgccaaaatgcaaaaagcacacaactgaaatctttatcatttgttattggagcagtaaatggcacgacgaaagaagctaaaagaacctacattcaagaacagtgtgaatttttggagaagttacgacaacaaaagataagagagggaagaattaacatattgtctatggatgctggtgtttctaactttgctttctctaagatgcaattgctcaataatgatccgctccctaaagtactagactggcaaaagataaatctagaggagaaattttttcaaaacctcaaaaagttaagcttgaatcctgctgaaacttctgagcttgtatttaaccttacggagtatttatttgaatctatgccgataccagatatgtttacaattgaaaggcaacgtaccagaactatgtcttcgaggcatattttagacccaattttaaaagtgaatattctcgaacagattcttttctctaacttggaaaataaaatgaagtatacgaataaaataccgaatacgtccaagttgaggtatatggtatgttcgtccgatccacatcggatgacttcatattggtgcattccaagagaagagacaccgaccagttcaaaaaagttaaaatctaacaaacatagcaaagattctcgaataaagctagtgaaaaaaatactttcaacctcaatactagaaggtaattcaactagttctacaaaactggtcgagttcataggagtttggaataataggataagaaatgcccttaccaaaaaaaaaagtttcaagctatgtgatatactagagatccaagataattcgggggtgagaaaagatgacgatttggcagattcattcctccattgtttgtcttggatggagtggttaaaaaattatgaaagtattactgaactcttgaattcaaaaacactggttaaaacacagttcggacaggtgtttgaattttgtgaaaataaggtacaaaagctgaaatttttgcagaacacttacaacaatgactaa

SEQ ID NO: 28 - DNA, 1800 bp, Arabidopsis thaliana
  source 1..1800: mol_type = genomic DNA
atgggtgtgggaggcaatttctgggatttgctgagaccatatgctcagcaacaaggctttgattttctcagaaacaaacgagtcgctgttgatctctccttctggatcgttcagcatgaaaccgctgttaagggtttcgtccttaaacctcacctccgactcactttcttccgtactatcaacctcttctcaaagtttggagcgtacccggtttttgtggttgatggaacaccatcacctttgaaatctcaggcgagaatctccaggtttttccgttcttctggaattgatacttgtaatctacctgtgattaaagatggtgtctcggttgagagaaacaagctgttttctgaatgggttagggaatgtgtggagctactcgaattgctcggtattccggtgctgaaagctaatggtgaggctgaagctctctgtgcacagttaaacagccaaggttttgtggatgcttgcattactcctgatagtgatgctttcctttttggtgctatgtgcgtgatcaaagacatcaagcctaattcaagagaaccttttgaatgctaccatatgtcacatatcgagtctggcctcggtttgaagcggaaacacttgattgctatttctctattggtgggaaacgattatgattcaggcggtgttcttgggattggtgtggataaagcactgcgcattgttcgtgagttttctgaagaccaagtacttgaaagactacaggacattggaaatgggttgcaacctgcagttcctggtggaatcaaatccggggatgatggtgaagaattccgctcagagatgaaaaaaagatctcctcactgttcccgttgtggacacctgggcagcaagagaactcattttaagtcctcttgtgagcactgcggttgtgatagtggttgcattaaaaaaccattagggtttagatgtgaatgctccttttgttccaaggatcgagatttaagggaacaaaagaaaaccaatgattggtggatcaaagtctgcgataagattgctctagcgccagagtttcccaacagaaagattattgaactttatctatccgatggtttgatgacaggagatggatcgtcaatgtcttggggaactcctgatactggaatgctagtggatctcatggttttcaaactgcactgggacccatcttatgttagaaaaatgttgcttccgatgctgtcgaccatttatctgagagaaaaggcaagaaacaacacaggatacgctttgttgtgtgatcaatacgaatttcattcaatcaagtgcataaaaactagatatgggcatcagtcctttgtaataaggtggagaaaacccaaatctacaagtggttatagtcatagtcacagcgagccagaagaatcaattgttgtattggaagaagaagaagagtctgttgatccgttggatggtttaaatgaacctcaggtgcaaaatgataatggtgactgcttcttgctaactgatgaatgcataggacttgttcagtctgctttccctgatgaaacagagcattttctacatgagaagaaactgagagagtcgaaaaagaagaatgtttctgaagaagaaacagcaacaccaagagcaacaacaatgggtgtacaaagaagcattaccgatttctaccgttcagcgaagaaagcagcagcaggtcaaagtatagagacaggcgggagttcaaaagcttctgcggaaaagaagagacaggcaacttctactagtagtagtaaccttacaaagtcggtcaggcgtcgtctcttgtttggatag

SEQ ID NO: 29 - DNA, 2675 bp, synthetic construct
  misc_feature 1..2675: note = Selection donor vector for introduction of premature stop codon into the tobacco PDS gene
  source 1..2675: mol_type = other DNA
cttcaggaagtttcccgctcaaaaacgtcacatcattcaacagtccccttccacgtgtcacgttttgattgggtgcccatttttttcgggccatccggtaatattataacggatggcccttatgaaagttgtactaatatatataatactccaatactccaatagatatgttaatatgctaagccacgtcatcggagtacaaacatggaattcctggctgatgctggtcacaaaccgatattgctggaggcaagagatgtcctaggtggaaaggtgaagaatatccatgctttcctttaattttattcctttttcttttgtgtccttccctattgatagtcccttttcaggaaggcttctatttgttttgtttaaaatcatttttcatactctttaaacattcagttgctcaaacaattgcaagggtgttcactattcctatttttgactgtcttcctttctctcagtttagttttattcctctctctctctctctctctatttttggaggaaatagatctgtcctaaaaatttccagctttactactaatagtgttaattgtcgagaaaatagtacagcatattaggtaaaagatatggaaagtatattattattattattattattattattattattattattattattattattattattattattattattattctctattattttaagattgagtcaattttacctgtcctgttggttgcatttctcatataaacattcttttctgtgagatgctatgtgaattagctgatgtttttggtatagagcactatgttagtcagttttatcttactgaagcagtcaccaagaatctagttgtataggctaaaagattgaattagcattaatctttatgtgttctgcacctgaatacttatacctaccttttaggtagctgcatagaaagatgatgatagagattggtatgagactgggttgcacatattctgtaagtttgactcctcaagaatgcatactttaatcttctagtacaacagtttctttcaagatctcttttgtccattaatcagatagctatccctgtttgtcttttgcaaatagccaatatgtcagtcgatctgtattctgccttgcctatctttttttatctgttaatttcgtatggtgactcatacaagttggtgcatctcctttaagttggggcttacccaaatatgcagaacttgtttggagaactagggataaacgatcggttgcagtggaaggaacattcaatgatatttgcgatgcctaacaagccagggggatcccgcagggaggcaaacaatgatatcacaactctcctgacgcgtcatcgtcggctacagcctcgggaattgctacctagctcgagcaagatccaaggagatataacaatggcttcctcctggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaactagtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggttttttgattagagtcccgcaattatacatttaatacgcgatagaaaacaaaatatagcgcgcaaactaggataaattatcgcgcgcggtgtcatctatgttactagatcgacccgggcttcaggaagtttcccgctcaaaaacgtcacatcattcaacagtccccttccacgtgtcacgttttgattgggtgcccatttttttcgggccatccggtaatattataacggatggcccttatgaaagttgtactaatatatataatactccaatactccaatagatatgttaatatgctaagccacgtcatcggagtaca

SEQ ID NO: 30 - DNA, 2619 bp, synthetic construct
  misc_feature 1..2619: note = BOR1-ALS LFS-nptII-ALS RFS BOR2
  source 1..2619: mol_type = other DNA
tcctgtactccgatgacgtggcttagcatattaacatatctattggagtattggagtattatatatattagtacaactttcataagggccatccgttataatattaccggatggcccgaaaaaaatgggcacccaatcaaaacgtgacacgtggaaggggactgttgaatgatgtgacgtttttgagcgggaaacttcctgaaggcgcgccggagagtaaggaaggtaaactgaagttggatttttctgcttggaggcaggagttgacggtgcagaaagtgaagtacccgttgaattttaaaacttttggtgatgctattcctccgcaatatgctatccaggttctagatgagttaactaatgggagtgctattataagtaccggtgttgggcagcaccagatgtgggctgctcaatattataagtacagaaagccacgccaatggttgacatctggtggattaggagcgatgggatttggtttgcccgctgctattggtgcggctgttggaagacctgatgaagttgtggttgacattgatggtgatggcagtttcatcatgaatgtgcaggagctagcaactattaaggtggagaatctcccagttaagattatgttactgaataatcaacacttgggaatggtggttcaattggaggatcggttctataaggctaacagagcacacacatacctggggaatccttctaatgaggcggagatctttcctaatatgttgaaatttgcagaggcttgtggcgtacctgctgcgagagtgacacacagggatgatcttagagcggctattcaaaagatgttagacactcctgggccatacttgttggatgtgattgtacctcatcaggaacatgttctacctatgattcccagtggcggggctttcaaagatgtgatcacagagggtgacagaagttcctatggatcccgcagggaggcaaacaatgatatcacaactctcctgacgcgtcatcgtcggctacagcctcgggaattgctacctagctcgagcaagatccaaggagatataacaatggcttcctcctggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaactagtgtttgagaagctacagagctagttctaggccttgtattatctaaaataaacttctattaaaccaaaaatgttatgtctattagtttgttattagtttttccgtggctttgctcattgtcagtgttgtactattaagtagttgatatttatgtttgctttaagttttgcatcatctcgctttggttttgaatgtgaaggatttcagcaatgtttcattctctattcgcaacatccagtcggtatccggagctctatgtagtatgtctggagattaatttctagtggagtagtttagtgcgataaagttagcttgttccacatttttatttcgtaacctgggtcagattggaacttcctctttaggttggatgcaatccctatttgggctttctcttaatttcattattgaaattgttggcttttaatctgagcaagttgatttgcagctttctctcttgagtcctagcgagcaatacgttatctctgtctcctatttcttagtggataatcttatgatggaaatctgtggagataggaaagcggccgctcctgtactccgatgacgtggcttagcatattaacatatctattggagtattggagtattatatatattagtacaactttcataagggccatccgttataatattaccggatggcccgaaaaaaatgggcacccaatcaaaacgtgacacgtggaaggggactgttgaatgatgtgacgtttttgagcgggaaacttcctgaagccg

SEQ ID NO: 31 - DNA, 3152 bp, synthetic construct
  misc_feature 1..3152: note = BOR1-PPOX1 LFS-nptII-PPOX1 RFS BOR2
  source 1..3152: mol_type = other DNA
tcctgtactccgatgacgtggcttagcatattaacatatctattggagtattggagtattatatatattagtacaactttcataagggccatccgttataatattaccggatggcccgaaaaaaatgggcacccaatcaaaacgtgacacgtggaaggggactgttgaatgatgtgacgtttttgagcgggaaacttcctgaagggcgcgccacaataacatcaggatttgattcatcttaaatataatacttgcgtttcatatgcaggaacgatatatagttcatcactcttccctaaccgtgccccaaaaggtcgggtgctactcttgaacatgattggaggagcaaaaaatccggaaattttgtctaaggtaaggcatttaaattgagcgcaacttcaagtcctgccggtattgatttatcataatgaatattcatgggctggccagttggggcatcgcttgaaggttcctatggtaacaaacagaaaactgagaatgaccttatcccctgatctcttttggttgcaaatacgttaaccttgcagtgatgaggagaatgttttcagtgttattttctctagattaatggcgtagttggtttggattacaaaaccgtttaggcttatatgtgattagagtctccaggtttttgatctagcagtaagagcaaaatgtgtgatgttttaaaaaaacagtcaagtgattacaaatactcagaatagtttcaccataaagaactaaagagctaagaagattgtggtttctctttcctctatgttcgactgcattttgtgggttttccatagcatgactttcttttatggtacttgtacgaaagcatttcgctgttattctttatgcttcagggcctagaataaattgctttgagatttgatccctcattcttcttgcagacggagagccaacttgtggaagtagttgatcgtgacctcagaaaaatgcttatagaacccaaagcacaagatccccttgttgtgggtgtgcgagtatggccacaagctatcccacagtttttggttggtcatctgggtacgctaagtactgcaaaagctgctatgagtgataatgggcttgaagggctgtttcttgggggtaattatgtgtcaggtgtagcattggggaggtgtgttgaaggagcttatgaagttgcatctgaggtaacaggatttctgtctcggtatgcttacaaaggatcccgcagggaggcaaacaatgatatcacaactctcctgacgcgtcatcgtcggctacagcctcgggaattgctacctagctcgagcaagatccaaggagatataacaatggcttcctcctggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaactagtaacctgtctgggggtactgctaggtccaaaccttgttagtaatacgatcatgccttgggaatattggcatgtgcctaaaagttttgctcgttagagttattttagccttggtaaatgatttgtacttgatatcagtcgttttctttgagataaaatgttcctgttcaggaaaatataatgtatatcaattttaaacacttgaatgttgaagatcattttttcccctcagcttacccataaatgtgaaaggtcctttgcttctgcatggtgagactgccgatatattttctccaacttcctatggttaaatatggtttgccttgtcattctttgttttctttgggagattatttattccacgaccagaagtaagggagtataccacattgattcaagggctgatacttgtggcaacaaagactaactgtgcaagggtaacagtgagagtatatttattactcacttctaataaagagcaaagttaagggaattcgatgatttaggagcaaattaggccttttcccttgagttaataaagagatgctattaaaattatacttctttgtatttaaatttgatataggtaatatgttctgtcatacaacatttattaggtgtaattcaatagtacttttaaaaaataaagttttttaacatgaaatcaaaaatagatcaggactgctcttgtatagatctagtctagttaaaataaataactgcataagtgtttgcccacactaatcatataggcacccaaaactctcctcctttttcgtccccaaacgtctcctccctcgctgcggccgctcctgtactccgatgacgtggcttagcatattaacatatctattggagtattggagtattatatatattagtacaactttcataagggccatccgttataatattaccggatggcccgaaaaaaatgggcacccaatcaaaacgtgacacgtggaaggggactgttgaatgatgtgacgtttttgagcgggaaacttcctgaagccg

SEQ ID NO: 32 - DNA, 791 bp, Escherichia coli
  source 1..791: mol_type = genomic DNA
atggaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctg

SEQ ID NO: 33 - DNA, 188 bp, synthetic construct
  misc_feature 1..188: note = Tobacco ALS sgRNA
  source 1..188: mol_type = other DNA
gatgtgatcacagagggtgacgttttagagctaggccaacatgaggatcacccatgtctgcagggcctagcaagttaaaataaggctagtccgttatcaacttggccaacatgaggatcacccatgtctgcagggccaagtggcaccgagtcggtgctttttttttttgcggccatcttgctgaaaaa

SEQ ID NO: 34 - DNA, 168 bp, synthetic construct
  misc_feature 1..168: note = Tobacco PPOX1 sgRNA
  source 1..168: mol_type = other DNA
ggctgcatggaaagatgatgagttttagagctaggccaacatgaggatcacccatgtctgcagggcctagcaagttaaaataaggctagtccgttatcaacttggccaacatgaggatcacccatgtctgcagggccaagtggcaccgagtcggtgcttttttttttt

SEQ ID NO: 35 - DNA, 1077 bp, synthetic construct
  misc_feature 1..1077: note = Tobacco PDS donor for knock out mutagenesis with premature stop codon in the PDS exon
  source 1..1077: mol_type = other DNA
ctggctgatgctggtcacaaaccgatattgctggaggcaagagatgtcctaggtggaaaggtgaagaatatccatgctttcctttaattttattcctttttcttttgtgtccttccctattgatagtcccttttcaggaaggcttctatttgttttgtttaaaatcatttttcatactctttaaacattcagttgctcaaacaattgcaagggtgttcactattcctatttttgactgtcttcctttctctcagtttagttttattcctctctctctctctctctctatttttggaggaaatagatctgtcctaaaaatttccagctttactactaatagtgttaattgtcgagaaaatagtacagcatattaggtaaaagatatggaaagtatattattattattattattattattattattattattattattattattattattattattattattattattctctattattttaagattgagtcaattttacctgtcctgttggttgcatttctcatataaacattcttttctgtgagatgctatgtgaattagctgatgtttttggtatagagcactatgttagtcagttttatcttactgaagcagtcaccaagaatctagttgtataggctaaaagattgaattagcattaatctttatgtgttctgcacctgaatacttatacctaccttttaggtagctgcatagaaagatgatgatagagattggtatgagactgggttgcacatattctgtaagtttgactcctcaagaatgcatactttaatcttctagtacaacagtttctttcaagatctcttttgtccattaatcagatagctatccctgtttgtcttttgcaaatagccaatatgtcagtcgatctgtattctgccttgcctatctttttttatctgttaatttcgtatggtgactcatacaagttggtgcatctcctttaagttggggcttacccaaatatgcagaacttgtttggagaactagggataaacgatcggttgcagtggaaggaacattcaatgatatttgcgatgcctaacaagccaggg

SEQ ID NO: 36 - DNA, 168 bp, synthetic construct; identical to SEQ ID NO: 34
  misc_feature 1..168: note = sgRNA for knock out mutagenesis of tobacco PDS gene
  source 1..168: mol_type = other DNA

SEQ ID NO: 37 - DNA, 4188 bp, synthetic construct
  misc_feature 1..4188: note = Mutated cas9 gene to generate dead nuclease (dCas9)
  misc_feature 28..30: note = Asp to Ala
  misc_feature 2518..2520: note = His to Ala
  source 1..4188: mol_type = other DNA
atggacaagaagtacagcatcggcctggccatcggcacgaactcggtgggctgggcggtgatcacggacgagtacaaggtgccctccaagaagttcaaggtgctgggcaacaccgaccgccactcgatcaagaagaacctgatcggcgccctgctgttcgactccggcgagaccgccgaggcgacgcgcctgaagcgcaccgcgcgtcgccgctacacgcgtcgcaagaaccgcatctgctacctgcaggagatcttcagcaacgagatggccaaggtggacgactcgttcttccaccgcctggaggagtccttcctggtggaggaagacaagaagcacgagcgccaccccatcttcggcaacatcgtggacgaggtggcctaccacgagaagtacccgacgatctaccacctgcgcaagaagctggtggacagcaccgacaaggcggacctgcgcctgatctacctggccctggcgcacatgatcaagttccgcggccacttcctgatcgagggcgacctgaaccccgacaactcggacgtggacaagctgttcatccagctggtgcagacctacaaccagctgttcgaggagaacccgatcaacgcctccggcgtggacgccaaggcgatcctgagcgcgcgcctgtccaagagccgtcgcctggagaacctgatcgcccagctgcccggcgagaagaagaacggcctgttcggcaacctgatcgcgctgtcgctgggcctgacgccgaacttcaagtccaacttcgacctggccgaggacgcgaagctgcagctgagcaaggacacctacgacgacgacctggacaacctgctggcccagatcggcgaccagtacgcggacctgttcctggccgcgaagaacctgtcggacgccatcctgctgtccgacatcctgcgcgtgaacaccgagatcacgaaggcccccctgtcggcgtccatgatcaagcgctacgacgagcaccaccaggacctgaccctgctgaaggcgctggtgcgccagcagctgccggagaagtacaaggagatcttcttcgaccagagcaagaacggctacgccggctacatcgacggcggcgcgtcgcaagaggagttctacaagttcatcaagcccatcctggagaagatggacggcacggaggagctgctggtgaagctgaaccgcgaggacctgctgcgcaagcagcgcaccttcgacaacggcagcatcccccaccagatccacctgggcgagctgcacgccatcctgcgtcgccaagaggacttctacccgttcctgaaggacaaccgcgagaagatcgagaagatcctgacgttccgcatcccctactacgtgggcccgctggcccgcggcaacagccgcttcgcgtggatgacccgcaagtcggaggagaccatcacgccctggaacttcgaggaagtggtggacaagggcgccagcgcgcagtcgttcatcgagcgcatgaccaacttcgacaagaacctgcccaacgagaaggtgctgccgaagcactccctgctgtacgagtacttcaccgtgtacaacgagctgacgaaggtgaagtacgtgaccgagggcatgcgcaagcccgccttcctgagcggcgagcagaagaaggcgatcgtggacctgctgttcaagaccaaccgcaaggtgacggtgaagcagctgaaagaggactacttcaagaagatcgagtgcttcgacagcgtggagatctcgggcgtggaggaccgcttcaacgccagcctgggcacctaccacgacctgctgaagatcatcaaggacaaggacttcctggacaacgaggagaacgaggacatcctggaggacatcgtgctgaccctgacgctgttcgaggaccgcgagatgatcgaggagcgcctgaagacgtacgcccacctgttcgacgacaaggtgatgaagcagctgaagcgtcgccgctacaccggctggggccgcctgagccgcaagctgatcaacggcatccgcgacaagcagtccggcaagaccatcctggacttcctgaagagcgacggcttcgcgaaccgcaacttcatgcagctgatccacgacgactcgctgaccttcaaagaggacatccagaaggcccaggtgtcgggccagggcgactccctgcacgagcacatcgccaacctggcgggctcccccgcgatcaagaagggcatcctgcagaccgtgaaggtggtggacgagctggtgaaggtgatgggccgccacaagccggagaacatcgtgatcgagatggcccgcgagaaccagaccacgcagaagggccagaagaacagccgcgagcgcatgaagcgcatcgaggaaggcatcaaggagctgggctcgcagatcctgaaggagcaccccgtggagaacacccagctgcagaacgagaagctttacctgtactacctgcagaacggccgcgacatgtacgtggaccaggagctggacatcaaccgcctgtccgactacgacgtggacgccatcgtgccccagagcttcctgaaggacgactcgatcgacaacaaggtgctgacccgcagcgacaagaaccgcggcaagagcgacaacgtgccgtcggaggaagtggtgaagaagatgaagaactactggcgccagctgctgaacgccaagctgatcacgcagcgcaagttcgacaacctgaccaaggccgagcgcggtggcctgtcggagctggacaaggcgggcttcatcaagcgccagctggtggagacccgccagatcacgaagcacgtggcgcagatcctggactcccgcatgaacacgaagtacgacgagaacgacaagctgatccgcgaggtgaaggtgatcaccctgaagtccaagctggtcagcgacttccgcaaggacttccagttctacaaggtgcgcgagatcaacaactaccaccacgcccacgacgcgtacctgaacgccgtggtgggcaccgcgctgatcaagaagtaccccaagctggagagcgagttcgtgtacggcgactacaaggtgtacgacgtgcgcaagatgatcgccaagtcggagcaggagatcggcaaggccaccgcgaagtacttcttctactccaacatcatgaacttcttcaagaccgagatcacgctggccaacggcgagatccgcaagcgcccgctgatcgagaccaacggcgagacgggcgagatcgtgtgggacaagggccgcgacttcgcgaccgtgcgcaaggtgctgagcatgccccaggtgaacatcgtgaagaagaccgaggtgcagacgggcggcttctccaaggagagcatcctgccgaagcgcaactcggacaagctgatcgcccgcaagaaggactgggaccccaagaagtacggcggcttcgactccccgaccgtggcctacagcgtgctggtggtggcgaaggtggagaagggcaagtccaagaagctgaagagcgtgaaggagctgctgggcatcaccatcatggagcgcagctcgttcgagaagaaccccatcgacttcctggaggccaagggctacaaagaggtgaagaaggacctgatcatcaagctgccgaagtactcgctgttcgagctggagaacggccgcaagcgcatgctggcctccgcgggcgagctgcagaagggcaacgagctggccctgcccagcaagtacgtgaacttcctgtacctggcgtcccactacgagaagctgaagggctcgccggaggacaacgagcagaagcagctgttcgtggagcagcacaagcactacctggacgagatcatcgagcagatctcggagttctccaagcgcgtgatcctggccgacgcgaacctggacaaggtgctgagcgcctacaacaagcaccgcgacaagcccatccgcgagcaggcggagaacatcatccacctgttcaccctgacgaacctgggcgccccggccgcgttcaagtacttcgacaccacgatcgaccgcaagcgctacacctccacgaaagaggtgctggacgcgaccctgatccaccagagcatcaccggcctgtacgagacgcgcatcgacctgagccagctgggcggcgactcccgcgcggacccgaagaagaagcgcaaggtgagcgctggaggaggtggaagcggaggaggaggaagcggaggaggaggtagc

(19) United States
(12) Patent Application Publication
(10) Pub. No.: US 2025/0051789 A1
(43) Pub. Date: Feb. 13, 2025

(54) Gene Targeting

(71) Applicant: Algentech SAS, Evry Cedex (FR)

(72) Inventors: Alexander Sorokin, Evry Cedex (FR); Isabelle Malcuit, Evry Cedex (FR); Florina Vlad, Evry Cedex (FR)

(73) Assignee: Algentech SAS, Evry Cedex (FR)

(21) Appl. No.: 18/665,659

(22) Filed: May 16, 2024

Related U.S. Application Data
(63) Continuation of application No. 15/733,848, filed on Nov. 30, 2020, now abandoned.

(30) Foreign Application Priority Data
Jun. 1, 2018 (EP) 18175623.0

(51) Int. Cl. C12N 15/82; C12N 9/22; C12N 15/10; C12N 15/90

(52) U.S. Cl. CPC C12N 15/8213; C12N 9/22; C12N 15/8274; C12N 15/902; C07K 2319/85; C12N 15/1024; C12N 2800/80; C12N 2820/00; C12Y 301/11003; C12Y 301/22004

(57) ABSTRACT

Methods, reagents and compositions for providing more accurate and reliable genetic modification are provided. In particular a nucleic acid encoding a fusion protein comprising an endonuclease domain and a binding domain for an origin of replication is described. Also provided are methods, reagents and compositions for in vivo genetic modification of the genome of a non-animal cell or organism. Furthermore, the present application relates to uses of the said methods, reagents and compositions for introducing desirable traits to non-animal organisms or ameliorating or removing non-desirable traits in these organisms including in the treatment of disease.

This application is a continuation that claims the benefit of priority and is entitled to the filing date pursuant to 35 U.S.C. § 120 of U.S. Non-Provisional patent application Ser. No. 15/733,848, filed on Nov. 30, 2020, a U.S. national stage application filed pursuant to 35 U.S.C. § 371 from International Patent Application Serial No. PCT/EP2019/064215, filed on May 31, 2019 which claims the benefit of priority and the filing date of European Patent Application Serial No. 18175623.0, filed on Jun. 1, 2018, the content of each of which is hereby incorporated by reference in its entirety.

Submitted as part of this patent application is a Sequence Listing filed as an XML file named UEALG1-0001USC1-SeqList_ST26.xml having a file size of 90,000 bytes and generated on May 16, 2024, the content of which is hereby expressly incorporated by reference in its entirety.

The present invention relates to methods, reagents and compositions for providing more accurate and reliable genetic modification. The invention further provides methods, reagents and compositions for in vivo genetic modification of the genome of a non-animal cell or organism and selection for modified clones/plants. Furthermore, the present invention relates to uses of the said methods, reagents and compositions for introducing desirable traits to non-animal organisms or ameliorating or removing non-desirable traits in these organisms including in the treatment of diseases and production of transgenic organisms.

In recent times genetic modification by way of random mutagenesis has given way to directed mutagenesis of particular nucleotide sequences using sequence-specific protein complexes.

Examples of such protein complexes include zinc-finger nucleases (ZFN), transcription activator-like effector nucleases (TALENs), complexes derived from the CRISPR-Cas9 system of Streptococcus pyrogenes and other bacteria, and CRISPR-Cpf1.

ZFNs and TALENs are both protein nucleases whose protein structure allows them to interact with and recognise a particular DNA sequence before cutting the DNA at a defined location. Thus cutting a particular DNA sequence requires a uniquely designed ZFN or TALEN protein.

In contrast, the CRISPR-Cas9 and CRISPR-Cpf1 systems use a single protein whose activity is directed by an RNA cofactor whose nucleotide sequence defines the location that the endonuclease will act at to produce a double strand break.

Thus, all of these protein complexes act by making a DNA double strand break at a predefined DNA sequence. This double strand break is then normally repaired by the non-homologous end joining (NHEJ) pathway.

Repair by NHEJ is highly efficient and rapid but is more error-prone than the alternative pathway for repair of DNA double-stranded breaks which is homology-directed repair (HDR). Consequently, a proportion of NHEJ pair of events will cause insertion or deletion of nucleotides at the break site. Such insertion or deletion events are known as ‘indels’.

Alternatively, larger genetic modifications are enabled by the presence of a donor DNA molecule in the vicinity of an artificially-created DNA double-stranded break. In this instance HDR of the induced DSB causes repair of the DSB with using the sequence of the donor molecule. In this way specific modifications can be made and short sequence insertions are also possible. One example of such a donor and a vector for producing large amounts of such donor molecules is disclosed in WO 2010/084331.

Homologous recombination proceeds in several distinct stages: the earliest step is processing of the DNA end to produce 3′ single-stranded DNA (ssDNA). Following 5′ strand resection, the 3′ ssDNA is bound by RecA-type recombinases that catalyze homologous pairing and DNA strand exchange. The 3′ end then primes DNA synthesis, and resolution of Holliday junctions or strand annealing between newly-synthesized ends results in repair of the initial DSB (Seitz et al. 2001, PMID: 11677683).

However, the efficiency of genetic modification using homology-dependent recombination (HDR) is low because most repair of double strand breaks proceeds via the more rapid NHEJ pathway.

Furthermore, while the above-mentioned protein complexes are directed to specific sequences their endonuclease activity has been known to act at other sites. Such “off-site breaks” are particularly a problem as NHEJ is more error prone.

Thus, there exists a need for alternative and preferably improved methods and reagents for sequence-specific modification of nucleic acid sequences and of DNA sequences in particular. Furthermore, there is a need for techniques and reagents that more reliably and efficiently yield the desired genetic modification. Additionally, there is a need for techniques and reagents that reliably allow insertion of longer DNA sequences at a pre-defined locus.

An object of the present invention is to provide reagents and techniques for using these reagents that offer alternatives and preferably allow more reliable, efficient and accurate modification and/or mutation of a target genome at specific loci within the genome.

There are provided herein proteins and protein-nucleic acid complexes that provide improved transformation efficiencies and methods for carrying out such transformations. Furthermore the methods, reagents and compositions herein may be used for introducing desirable traits to plants, algae, bacteria and other non-animal organisms or ameliorating or removing non-desirable traits in these organisms including in the treatment of diseases.

Targeting of donor DNA to the target is a critical factor for homology-dependent recombination (HDR). A number of methods have been developed for donor DNA tethering to the target (Sharma & Mclaughlin, 2002, doi: 10.1021/ja020500n; Aird et al., 2018, doi: 10.1038/s42003-018-0054-2; Savic et al., 2018, doi: 10.7554/eLife.33761). Interestingly, covalent linking of donor DNA to cas9 fusion protein increases efficiency of homology-dependent recombination by 24-30 fold, as indicated by fusion of HUH endonucleases to cas9 (Aird et al., 2018, doi: 10.1038/s42003-018-0054-2) or cas9-SNAP-tag domain fusion (Savic et al., 2018, doi: 10.7554/eLife.33761).

Tethering of proteins to RNAs by bacteriophage proteins has been established for decades (Baron-Benhamou et al., 2004, doi: 10.1385/1-59259-750-5-135; Coller & Wickens, 2007, doi: 10.1016/S0076-6879(07)29014-7; Keryer-Bibens et al., 2008, doi: 10.1042/BC20070067; Tsai et al., 2011, doi: 10.1074/mcp.M110.007385), and such approaches could be also utilised for tethering of donor DNA by DNA binding proteins fused to bacteriophage coat proteins recognising specific stem-loop RNA structures. A number of stem-loops and bacteriophage coat proteins are available for tethering, such as MS2 stem loop-MS2 coat protein (Peabody, 1993, PMID: 8440248), PP7 stem loop-PP7 coat protein (Lim & Peabody, 2002, PMID: 12364592), B-box stem loop-lambda N coat protein (Keryer-Biben et al., 2008, doi: 10.1042/BC20070067).

Tethering customized sgRNA from CRISPR with the bacteriophage coat protein-binding RNA stem-loop was described, where stem-loop RNA structure were introduced inside or at the 3′ end of sgRNA and a potential protein of interest was fused to bacteriophage coat protein (Konenmann et al., 2015, doi: 10.1038/nature14136; Nowak et al., 2016, doi: 10.1093/nar/gkw908; Park et al., 2017, doi: 10.1371/journal.pone0179410; Anton et al., 2018, doi: 10.1093/biomethods/bpy002) for site-specific visualization of genomic elements, transcriptional regulation and epigenetic manipulation.

The tethering of donor DNA to the target is, however, technically challenging, as (i) single-stranded linear DNA (SSIDNA) should be produced in vitro, (ii) ssIDNA delivered to cells is less efficient then dsDNA, (iii) ssIDNA is not stable in vivo and is subject to rapid endonuclease degradation, and as result, (iv) low concentration of donor DNA around the targeted locus significantly reduces HDR.

Thus delivery of ssDNA to cells is challenging. ssDNA is difficult to deliver technically because ssDNA is not naturally imported into cells and is rapidly degraded. Advantageously, the present invention addresses this problem by delivering dsDNA and then producing ssDNA in the desired location from this dsDNA.

To address these issues we utilise e.g. HUH rep proteins from bacteriophages, circoviruses, geminiviruses, rolling circle transposons from bacteria or plants (such as helitrons) preferentially active in plant cells for rolling circle replication, and replicative donor vector containing double-stranded donor DNA flanked by one or two viral origins of replication.

Modification of the target is significantly improved by producing ssDNA in vivo and causing it to accumulate in the vicinity of the locus to be modified. Accumulating the ssDNA in the vicinity of the locus to be modified means that it is available for use in HDR processes for a longer period, which advantageously promotes HDR. Additionally, amplification of the ssDNA copy number allows more of the ssDNA moiety to accumulate close to the locus of interest, which, as noted above, promotes more efficient editing of the target locus.

Our approach allows addressing all problems indicated above by one or more or all of:
(i) producing single-stranded linear (ssIDNA) or single-stranded circular DNA (sscDNA) in cells in vivo from double-stranded DNA, e.g. the more stable double-stranded DNA (dsDNA) of the replicative donor vector:
(ii) tethering of ssIDNA to the target by covalent linkage of donor DNA with rep protein fused to endonuclease, e.g. cas9, or to a bacteriophage coat protein (e.g. MS2 coat protein) in combination with a stem-loop RNA structure (e.g. MS2 stem-loop) introduced into sgRNA;
(iii) enhancing donor DNA accumulation near a targeted locus providing an excess of donor DNA for a longer period of time.

Single-stranded donor DNA can be produced from a linear dsDNA donor replicative vector with one origin of replication fused to the 5′ end of donor DNA, or from a linear or circular dsDNA replicative vector where a donor DNA fragment is flanked by origins of replication on both 5′ and 3′ ends.

Accordingly, the present invention provides a nucleic acid encoding a first fusion protein comprising an endonuclease or bacteriophage coat protein domain and a binding domain for an origin of replication.

Functionally significant domains or regions of different proteins or polypeptides may be combined for expression from an encoding nucleic acid as a fusion protein. For example, particularly advantageous or desirable properties of different proteins or polypeptides may be combined in a hybrid protein, such that the resultant expression product may include fragments of various parent proteins or polypeptides.

In the fusion proteins described herein the domains of the fusion proteins are preferably joined together via linker peptides. The particular choice of linker will depend on the constituent domains of the fusion protein. The suitability and choice of appropriate linker peptides is discussed in Chen et al. (Adv Drug Deliv Rev. 2013; 65 (10): 1357-1369).

The endonuclease may cleave a target nucleic acid molecule in a sequence specific manner. The sequence specific cleavage of the nucleic acid molecule may be double or single stranded (including ‘nicking’ of duplexed nucleic acid molecules; double stranded cleavage may yield blunt ends or overhanging termini (5′ or 3′ overhangs)). The sequence specific nuclease preferably acts as a monomer but may act as a dimer or multimer. For instance a homodimer wherein both monomers make single strand nicks at a target site can yield a double-strand break in the target molecule. Preferably the cleavage event makes a double-stranded break in the target molecule.

Examples of sequence-specific endonucleases include zinc-finger nucleases (ZFN), transcription activator-like effector nucleases (TALENs), complexes derived from the CRISPR-Cas9 system of Streptococcus pyogenes and other bacteria, and CRISPR-Cpf1.

A nucleic acid molecule may comprise double-or single-stranded DNA or RNA. The nucleic acid molecule may also comprise a DNA-RNA duplex. Preferably the nucleic acid molecule is double-stranded DNA. Preferably the cleavage event makes a double-stranded DNA break in the target molecule.

Preferably the endonuclease is a DNA endonuclease and most preferably this is Cas9. This may be Cas9 from Streptococcus pyrogenes or a homologous or functionally equivalent enzyme from another bacteria.

The fusion protein may comprise an endonuclease and a component of the replication initiation complex or replication complex.

The components of the replication initiation complex or replication complex are necessarily associated with origins of replication and may be covalently attached thereto or to the elongating nucleic acid molecule. Suitably the origin of replication is derived from bacteriophages, eukaryotic viruses and various types of transposons, maintaining rolling circle replication function in the targeted cell. The endonuclease for specific origin of replication may first produce a stem loop at dsDNA origin fused to donor, nick single-stranded DNA at the stem loop followed by formation of a covalent phosphotyrosine intermediate, whereby the 5′ end of the DNA strand becomes linked to a specific tyrosine in the HUH-protein. Most suitably for application in plants are geminivirus, plant rolling circle transposons or another family of the rep genes.

Geminivirus Rep protein (GV-Rep) binds to the geminivirus origin of replication and thus becomes covalently linked to the ssDNA strand of donor DNA produced by rolling circle replication initiated at the origin of replication. Thus the newly replicated donor DNA molecule is covalently linked to the first fusion protein and is necessarily brought into close proximity to the site of the double-stranded DNA break caused by the endonuclease.

The invention further provides a nucleic acid encoding a second fusion protein comprising a 5′ to 3′ DNA exonuclease domain and an RNA binding domain. As an alternative to the second fusion protein, the invention also provides for the use of a 5′ to 3′ DNA exonuclease without an RNA binding domain.

Both Zalatan et al. (Cell (2015) 160, 339-350) and the CRISPRainbow system described initially by Ma et al. (Nat Biotechnol. 2016 Apr. 18. doi: 10.1038/nbt.3526) utilise a modified sgRNA containing 3′ RNA hairpin aptamers that bind uniquely labelled RNA binding proteins (SEQ ID NO: 16). Thus the sgRNA is functionalised so that it can be used to locate fusion proteins comprising binding domains for the aptamers in association with the sgRNA and hence the endonuclease it is associated with.

The action of the second fusion protein may be for inhibition of NHEJ during transformation of a cellular genome so as to promote HDR. The effect of such 5′ to 3′ resection on DNA double-strand breaks is to suppress religation of DNA breaks (i.e. by blocking NHEJ), by producing a substrate that is less suitable for NHEJ but is significantly more suitable for HDR. The action of the second fusion protein may be for inhibition of NHEJ during transformation of a genome so as to promote HDR.

The exonuclease may be a dsDNA exonuclease. The exonuclease may be lambda exonuclease (λ-exo). Lambda exonuclease (SEQ ID NO: 15) is a 5′ to 3′ exonuclease and is involved in recombination, double-strand break repair, the MMS2 error-free branch of the post replication repair (PRR) pathway and DNA mismatch repair.

Lambda exonuclease (λ exo) plays an important role in the resection of DNA ends for DNA repair. Lambda exonuclease is a 5′-3′ exonuclease that progressively digests one strand of a duplex DNA molecule to generate a 3′-single stranded-overhang (Carter& Radding, 1971, PMID: 4928646). Because of its robust properties and low cost, λ exo is widely used in multiple biotechnology applications, such as genetic engineering using homologous recombination.

In the complex with DNA, λ exo unwinds two bases at the 5′ end of the substrate strand to pull it into the reaction centre. It hydrolyses double-stranded DNA (dsDNA) 130 times faster than single-stranded DNA (ssDNA) (Little, 1967, PMID: 6017737). A DNA duplex with a 5′ phosphorylated blunt or recessed end is the appropriate substrate for λ exo, while the digestion rate of a dsDNA with a 5′ hydroxyl end or a 5′ phosphorylated overhang is significantly slower (Mitsis & Kwagh, 1999, PMID. 10454600, Tongbo et al., 2018, doi: 10.1093/nar/gkyl54).

Exonucleases with 5′-3′ activities are presented in other organisms. The Cas4 protein is one of the core CRISPR-associated (Cas) proteins implicated in the prokaryotic CRISPR system for antiviral defence. The Cas4 protein is a 5′ to 3′ single stranded DNA exonuclease in vitro and it is involved in DNA duplex strand resection to generate recombinogenic 3′ single stranded DNA overhangs (Zhang et al., (2012) https://doi.org/10.137/journal.pone.0047232).

RecJ from Deinococcus radiodurans, a member of DHH family proteins, is the only 5′ nuclease involved in the RecF recombination pathway, providing the resection of DNA strand with a 5′ end at double-strand breaks as an essential step in recombinational DNA repair. As a processive nuclease. RecJ only degrades ssDNA in a 5′-3′ direction but nuclease alone is capable of digesting DNA with only 5′-ssDNA overhang (Jiao et al., 2012, doi. 10.1016/j.dnarep.2011.11.008).

Genetic studies in Saccharomyces cerevisiae show that end resection takes place in two steps. Initially, a short oligonucleotide tract is removed from the 5′ strand to create an early intermediate with a short 3′ overhang by the highly conserved Mre11-Rad50-Xrs2 (MRX) complex and Sae2. Then in a second step the early intermediate is rapidly processed generating an extensive tract of ssDNA by the exonuclease Exo1 and/or the helicase-topoisomerase complex Sgs1-Top3-Rmi1 with the endonuclease Dna2 (Mimitou& Symington, 2011, doi: 10.1016/j.dnarep.2010.12.004).

In archaea, such as Pyrococcus furiosus the end resection is executed by the bipolar helicase HerA and the 5′-3′ exonuclease NurA (Hopkins&Paull, 2008, doi: 10.1016/j.cell.2008.09.054). Thus, loading or activation of HerA-NurA complex promotes resection of the 5′ strand of the double-stranded DNA break (DSB) and initiate of strand invasion.

For more information on enzymes involved in 5′ end DNA resection and mechanisms of 3′ DNA ends generation in the three domains of life see Blackwood et al., 2013, (doi: 10.1042/BST20120307); Liu&Huang, 2016, (doi: 10.1016/j.gpb.2016.05.002); Raynard et., 2019, (doi/10.1101/gad.1742408); Sharad&You, 2016, (doi: 10.1093/abbs/gmw043); Yin&Petes, 2014, (doi.org/10.1534/genetics. 114.164517).

The invention further provides a nucleic acid encoding a third fusion protein comprising a recombination inducing domain and an RNA binding domain.

The recombination domain may be a protein or polypeptide that interacts with a target or donor nucleic acid molecule in order to catalyse modification of the nucleotide sequence of the target nucleic acid with reference to the nucleotide sequence of the donor nucleic acid molecule.

Modification of the target nucleic acid may be by way of insertion of all or a part of the sequence of the donor nucleic acid molecule or substitution of all or a part of the sequence of the donor nucleic acid molecule for a homologous section of the target nucleic acid molecule. In this way deletions, insertions, frameshift mutations and single nucleotide mutations may be achieved.

The recombination inducing event caused or mediated by the recombination inducing domain may be initiating or catalysing strand exchange between the target and donor nucleic acid molecules.

The recombination domain may be RecA from E. coli or a homologue thereof, Rad51 or a homologue thereof from a plant or another organism, or an annealase from such as bacteriophage λ recombination protein beta (BET; Redβ) or a homologue thereof. Studies of phage lambda in vivo have indicated that bacteriophage λ beta protein can catalyse steps that are central to both the strand annealing and strand invasion pathways of recombination (Matsubara et al., 2013, doi: 10.1371/journal.pone.0078869). A homologous protein in this case may have functional or sequence homology, preferably functional homology.

Preferably the recombination domain is a trimer of RecA (SEQ ID NO: 17) or Rad51 monomers (SEQ ID NO: 18). Most preferably the monomers are joined by peptide linkers. Use of a trimer of monomers for the recombination domain is advantageous because this allows binding of a turn of the nucleic acid helix in order to more efficiently initiate strand exchange and hence HDR.

The invention further provides a nucleic acid encoding a fourth fusion protein comprising a domain comprising an inhibitor of the mismatch repair pathway; and an RNA binding domain.

MSH2 and MSH6 are proteins involved in base mismatch repair and the repair of short insertion/deletion loops. The MSH2 dominant-negative mutant (Sia et al., 2001, doi: 10.1128/MCB.21.23.8157-8167.2001) (SEQ ID NO: 25) competes with MSH2 binding to mismatches thus blocking the ability of the wild-type MSH2 protein to repair these mismatches. A dominant negative allele of MSH6 is also known and may be used in the same way as the dominant negative allele of MSH2 (Bowers et al., 1999, doi: 10.1074/jbc.274.23.16115).

The invention further provides a nucleic acid encoding a fifth fusion protein comprising a domain comprising a Holliday junction resolvase and an RNA binding domain. Suitable resolvases are e.g. a bacteriophage T4 endonuclease VII (T4E7) (SEQ ID NO: 26) a bacteriophage T7 endonuclease I (Babon et al., 2003, doi: 10.1385/MB:23:1:73); CCE1 (SEQ ID NO: 27) a YDC2 resolvase from yeast (Kleff et al., 1992, PMCID:PMC556502; White et al., 1997, doi:10.1128/MCB.17.11.6465); a GEN1 resolvase from human (Ip et al., 2008, doi: 10.1038/nature07470), and an AtGEN1 resolvase from Arabidopsis thaliana (SEQ ID NO: 28), (Bauknecht & Kobbe, 2014, doi: 10.1104/pp.114.237834).

The rearrangement and repair of DNA by homologous recombination involves the creation of Holliday junctions, which are cleaved by a class of junction-specific endonucleases to generate recombinant duplex DNA products.

The formation of DNA joint molecules is a transient process, which usually disrupted at an early stage by anti-recombinogenic helicases such as Srs2, Mph1 or RTEL1 (Gangloff et al., 1994, PMCID: PMC359378; Malkova et al., 2003, PMCID: PMC4493758; Prakash et al., 2009, doi: 10.1101/gad.1737809).

In somatic cells HDR is suppressed by low expression of resolvase and high activities of anti-recombinogenic helicases. The DNA helicase that translocates along single-stranded DNA in the 3′ to 5′ direction displaces annealed DNA fragments and removes Holliday junction intermediates from a crossover-producing repair pathway, thereby reducing crossovers and HDR (Malkova et al., 2003, PMCID: PMC4493758).

In order to improve efficiency of HDR, timely delivery of resolvase to Holliday junctions, formed during donor DNA annealing, may thus be provided to fix the recombination event and translate it into the modification at the target site.

The second, third, fourth and fifth fusion proteins may bind to the RNA component of an RNA-guided endonuclease for use in transformation mediated by the RNA-guided endonuclease. Preferably an RNA component is a tracrRNA molecule or domain for use in transformation using the CRISPR-Cas9 system. Note that reference throughout to a given domain comprising, say, a RNA binding domain includes the given domain both being and comprising that specified domain.

The invention also provides a method of transforming the genome of a non-animal cell comprising the steps of:
a. expressing an RNA-guided endonuclease in the cell or introducing the RNA-guided endonuclease into the cell;
b. expressing in the cell or introducing into the cell a sequence specific guide RNA to direct cleavage by the endonuclease domain to a specific locus; and
c. expressing in the cell the nucleic acid encoding the second fusion protein or introducing the second fusion protein into the cell.

Thus the invention provides a system with multiple features that may be used separately or in concert. These features include one or more or all of:
a. Induction of dsDNA break using the sequence-specific endonuclease of the first fusion protein.
b. Amplification and delivery of donor nucleic acid molecule to within close proximity of the induced DNA break by associating the donor nucleic acid molecule with the origin-binding domain of the first fusion protein.
c. Suppression of repair of DNA breaks using non-homologous end joining (i.e. blocking NHEJ), preferably, as noted above, by 5′ to 3′ resection of double-stranded DNA breaks in order to produce a substrate that is not suitable for NHEJ but is more suitable for HDR.
d. Delivery of recombinase to the induced dsDNA break.
e. Suppression of the mismatch repair pathway in the vicinity of the induced dsDNA breaks by providing an inhibitor of this pathway. As noted above this is preferably a fusion protein comprising a dominant negative suppressor protein of the mismatch repair system.
f. Resolution of Holliday junctions induced by interaction between donor and target DNA.

Features (c), (d), (e) and (f) are suitably supplied to the HDR complex by their being provided in the form of the second, third, fourth and fifth fusion proteins, i.e. each comprises a domain that binds to an aptamer engineered to be part of the sgRNA that guides the endonuclease activity of the first fusion protein (e.g. the sgRNA of SEQ ID NO: 16).

The second, third, fourth and fifth fusion proteins each suitably comprises a domain that binds to an aptamer engineered to be part of the sgRNA that guides the endonuclease activity of an RNA-guided endonuclease. Therefore the second, third, fourth and fifth fusion proteins may be used in concert with an RNA-guided endonuclease other than the first fusion protein, such as Cas9 or Cpf1.

Feature (b) may also be provided comprising a domain that binds to an aptamer engineered to be part of the sgRNA that guides the endonuclease activity of an RNA-guided endonuclease.

One advantage flowing from use of any or all of the first, second, third, fourth and/or fifth fusion proteins of the invention is more reliable and efficient genetic modification.

A further advantage is that use of any or all of the first, second, third, fourth and/or fifth fusion proteins of the invention allows for insertion of longer DNA sequences at a locus or loci acted on by a sequence-guided endonuclease that has previously been reported.

The invention also provides a method of modifying the genome of a non-animal organism or cell comprising:
a. expressing in the cell the nucleic acid encoding the first fusion protein or introducing the first fusion protein into the cell; and
b. expressing in the cell or introducing into the cell a donor nucleic acid molecule comprising an origin of replication.

As will be appreciated, the first fusion protein comprises an endonuclease domain and a binding domain for an origin of replication, wherein the binding domain suitably matches, e.g. binds to, the origin of replication of the donor nucleic acid.

Advantageously, the first fusion protein is capable of performing multiple functions. These functions include one or more of, or all of:
production of ssDNA of donor from dsDNA;
amplification of donor DNA;
tethering of donor DNA to the target; and
accumulation of donor DNA in close proximity to the target.

Particular advantage(s) are yielded by amplifying donor DNA and/or accumulating this in close proximity to the target: accumulation of donor DNA near the locus of the DNA double-strand break promotes repair of the break by HDR. Providing a greater concentration of donor DNA near the target locus promotes HDR. Without wishing to be bound by theory, this is believed to be because the greater availability of a donor with a section homologous to the target means that the less accurate but quicker NHEJ pathway is not favoured under these conditions.

Non-animal organisms in the context of the present disclosure may be prokaryotes (bacteria and archaea), algae, plants or any other non-animal organism including protists and fungi. Preferably, the non-animal organisms are plants along with any part or propagule thereof, seed, selfed or hybrid progeny and descendants. The plants may be monocot or dicot plants. Suitably the plants are Arabidopsis, tobacco, rice or a transgenic crop plant. Examples of suitable transgenic crop plants include tobacco (Nicotiana tabacum) and other Nicotiana species, carrot, vegetable and oilseed Brassicas, melons, Capsicums, grape vines, lettuce, strawberry, sugar beet, wheat, barley, (corn) maize, rice, soya bean, peas, sorghum, sunflower, tomato, cotton, and potato. The non-animal organisms may be algae.

The donor nucleic acid molecule may comprise:
a. a donor nucleic acid sequence;
b. a viral origin of replication sequence located at 5′ end of the donor nucleic acid sequence,
c. viral origins of replication both at 5′ and 3′ ends of the donor DNA sequence, or
d. a viral origin of replication at 5′ end of the donor DNA sequence, and a replication terminator at 3′ end of the donor DNA sequence.

The replication terminator may be a non-functioning origin of replication that is still capable of terminating replication when a replication fork reaches it. It is optionally omitted if linear dsDNA donor is flanked at 5′ end by viral origin of replication. In a specific example, a geminivirus origin of replication is nicked by the Rep protein at a particular location on a stem loop characteristic of the origin of replication. As long as the stem loop is present and correctly nicked then replication may be terminated at that location. Other sequence elements of the origin are not essential for termination and therefore can be omitted from the replication terminator in this example.

However, the nick at the replication terminator derived from such an origin of replication (in, for instance geminiviruses) may still be competent for religation of the nicked stem loops at the active origin of replication and the downstream terminator/origin of replication. In this way a nucleic acid circle with an active origin of replication is provided and may be actively replicated by rolling circle replication or another mode of replication.

Rolling circle replication of the donor DNA acid molecule has the advantage of providing a large amount of donor DNA nucleic acid. Provision of a relatively large amount of donor nucleic acid molecule means that the probability of the successful transformation is raised.

Although modification in desirable locus of the cells can be introduced, recovery of modified clones or plants from such cells is difficult due to competition between modified and non-modified cells. Regeneration of clones or plants from the population of modified and non-modified cells can be tedious and time-consuming.

The method provides a specific replicative donor vector allowing selection for clones/plants with desirable modification.

Accordingly, also provided by the invention is a selection vector comprising first and second viral origins of replication, wherein the first and second viral origins of replication are arranged to flank a donor DNA fragment; and the donor DNA fragment comprises a selectable marker gene that is fused out of frame.

The first and second viral origins of replication may be arranged to flank a DNA sequence comprising a promoter and a donor DNA fragment, and the donor DNA fragment may comprise a selectable marker gene that is out of frame with the promoter.

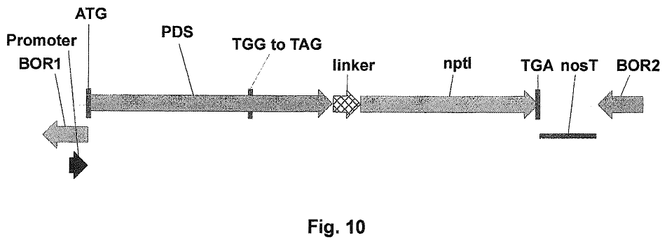

One example of the selection vector for introduction of knock out mutation in the cell and recovery of clones or plants on selection media is presented in Example 3 and FIG. 10.

The introduced selection vector comprises more generally two viral origins of replication flanking a donor DNA fragment and a selectable marker gene fused in frame with respect to translation of the product giving rise to the effect of the marker. The viral origin of replication at the 5′ end of the donor DNA contains a eukaryotic promoter with an ATG translation codon, fused ‘in-frame’ with donor DNA fragment, linker, selectable marker gene (such as nptII, hygromycin or phosphinotricin resistance genes) terminator (such as the nos terminator), followed by 3′ end viral origin of replication (SEQ ID NO: 29). All sequences introduced after the ATG codon represent one translational unit, generating a selectable marker, e.g. resistance to antibiotic, in this example: kanamycin antibiotic.

In order to introduce a knock out mutation into specific gene, a stop codon/deletion/insertion is introduced in the donor DNA fragment. As the stop codon is introduced into the donor fragment in front of a selectable marker gene, which is preferably nptII, no antibiotic resistance generated by the selection donor vector is observed due to premature termination of translational unit on selection vector.

Recombination of the donor DNA fragment with the target transfers the stop codon to the target sequence, while the DNA fragment without a stop codon from the target replaces the donor fragment in the selection donor vector. As result, the translational unit on the donor vector will be restored, and the vector will be amplified, allowing selection on kanamycin supplemented medium. The cells where translational unit of the donor vector was restored by exchange between donor and target DNA strands during recombination process will be resistant to kanamycin selection, and clones or plants can be recovered from such cells on selection medium.

The methods described herein may comprise introducing a double strand break into the genome in the presence of an exogenous donor nucleic acid molecule comprising a donor nucleic acid sequence as a template for modifying the genome or as an exogenous sequence to be integrated into the genome; a DNA repair mechanism modifies the genome via homology-directed repair (HDR).

The method may further comprise the step or effect of suppressing non-homologous end joining (NHEJ) repair of a DNA double-strand break to promote repair of the break by HDR by expressing in the cell a nucleic acid encoding the second fusion protein or introducing the second fusion protein into the cell.

The methods described herein may comprise introducing a double strand break into the genome in the presence of an exogenous donor nucleic acid molecule comprising a donor nucleic acid sequence as a template for modifying the genome or as an exogenous sequence to be integrated into the genome, and wherein a DNA repair mechanism modifies the genome via homology-directed repair (HDR), the method comprising:
suppressing non-homologous end joining (NHEJ) repair of the break to promote repair by HDR by expressing in the cell a nucleic acid encoding the second fusion protein or introducing the second fusion protein into the cell.

The method may further comprise the steps of:
a. expressing in the cell or introducing into the cell a sequence specific guide RNA to direct cleavage by the endonuclease domain to a specific locus; and
b. expressing in the cell one or more nucleic acids of the second, third, fourth and fifth fusion proteins or introducing one or more of the second, third, fourth and fifth fusion proteins into the cell.

The method may further comprise the steps of:
a. expressing in the cell one or more nucleic acids encoding the second, third, fourth and fifth fusion proteins; or
b. introducing into the cell one or more of the second, third, fourth and fifth fusion proteins.

The method may further comprise the steps of expressing in the cell two or more nucleic acids encoding the second, third, fourth and fifth fusion proteins or introducing into the cell two or more of the second, third, fourth and fifth fusion proteins, wherein the RNA binding protein domains of the respective fusion proteins bind to different RNA sequences.

In this way the first fusion protein may be using in concert with the second, third, fourth and fifth fusion proteins for transformation of a non-animal cell or organism in concert with an RNA-guided endonuclease.

Expression of the first, second, third, fourth and fifth fusion proteins during a method of modifying the genome as described herein may be via inducible and/or transient expression.

Various methods for introducing nucleic acids encoding the fusion proteins and nucleic acids of the invention are envisaged; these include electroporation and infiltration in order to introduce proteins, DNA and/or RNA. Also envisaged is the use of delivery systems, including liposomes or lipid nanoparticles (LNP), for directly introducing proteins, DNA and/or RNA, preferably by encapsulation of the proteins, DNA and/or RNA therein.

The invention also provides a first fusion protein comprising an endonuclease and a component of the replication initiation complex or replication complex.

The component of the replication initiation complex or replication complex may be also introduced in fusion with bacteriophage coat protein (MS2 coat protein) in combination with stem loops introduced into the sgRNA of CRISPR system.

The invention further provides a second fusion protein comprising a 5′ to 3′ DNA exonuclease domain and an RNA binding domain.

The invention also provides a third fusion protein comprising a recombination inducing domain and an RNA binding domain.

The invention further provides a fourth fusion protein comprising a domain comprising an inhibitor of the mismatch repair pathway and an RNA binding domain.

The invention further provides for use of the first fusion protein, or a nucleic acid encoding the first fusion protein in transformation of a non-animal organism or cell.

The invention further provides for use of the second fusion protein, or a nucleic acid encoding the second fusion protein in transformation of a non-animal organism or cell using an RNA-guided endonuclease.

The invention also provides for use of the first fusion protein, or a nucleic acid encoding the first fusion protein in concert with the second, third and fourth fusion proteins in transforming a non-animal organism or cell using an RNA-guided endonuclease.

The invention provides for use of the first fusion protein, or a nucleic acid encoding the first fusion protein in concert with the second, third, fourth and fifth fusion proteins in transforming a non-animal organism or cell using an RNA-guided endonuclease.

The invention provides for use of the first fusion protein, or a nucleic acid encoding the first fusion protein in concert with combination of any fusion protein(s) (second, third, fourth and/or fifth) dependent on desirable gene modification in transforming a non-animal organism or cell using an RNA-guided endonuclease.

The invention further provides vectors comprising the nucleic acids of the invention. Such vectors may be suitable for modification in vitro or in vivo and selection for modified clones and plants.

Vectors of the invention capable of expressing products encoded on nucleotides of the invention may also be suitable for expression in a host cell or cell-free system. Suitably the host cell may be a cultured plant cell, yeast cell or bacterial cell, e.g. Escherichia coli. Compositions and products of the invention may be obtained by methods comprising expressing such encoded products in a suitable host cell or cell-free system.

The invention also provides the methods, reagents and compositions disclosed herein for use in the treatment of disease in non-animal organisms.

The invention also provides uses of the methods, reagents and compositions disclosed herein for introducing desirable genetic characteristics to non-animal organisms or ameliorating or removing non-desirable genetic characteristics in these organisms.

The invention also provides uses of the methods, reagents and compositions disclosed herein for introducing desirable heritable characteristics to non-animal organisms or ameliorating or removing non-desirable inherited characteristics in these organisms.

Accordingly, the invention also provides non-animal transgenic organisms, transgenic cells thereof and transgenic non-animal cell lines. Organisms which include a transgenic cell according to the invention are also provided.

The invention further provides methods of treating disease or other conditions of non-animal organisms or cells by utilising the methods, reagents and compositions disclosed herein.

The invention is now illustrated in specific embodiments with reference to the accompanying drawings in which:

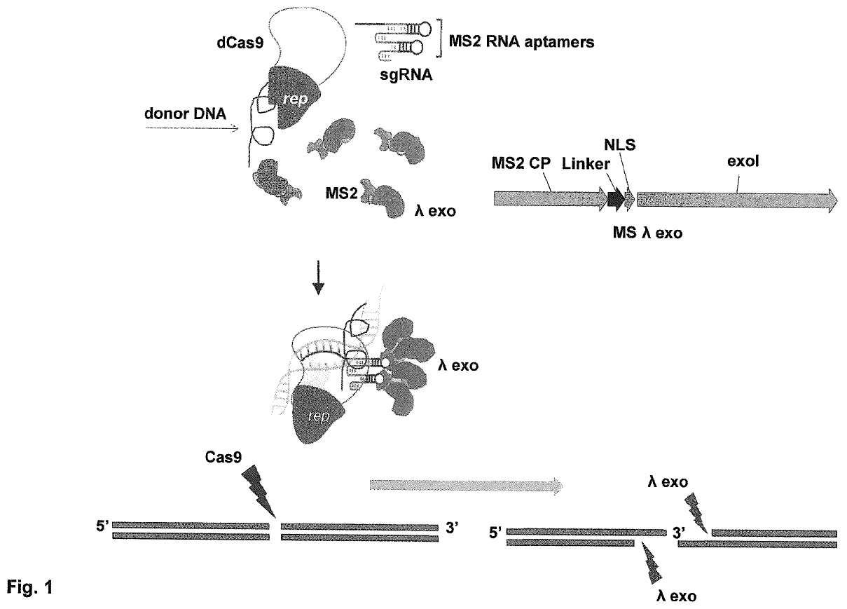

FIG. 1 shows a schematic representation of inducing a DNA double strand break with Cas9 protein (Cas9) and resecting the DNA DSB with exo1. The exo1-MS2 fusion protein is engineered to bind to the single guide RNA (sgRNA) via aptamer loops on the sgRNA that bind to the MS2 domain (part of SEQ ID NO: 22).

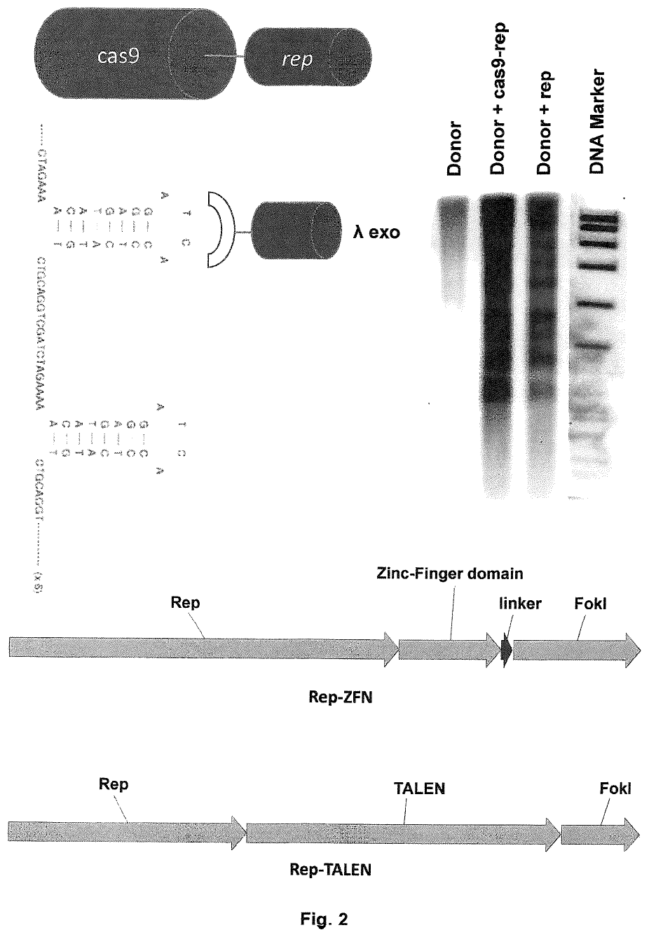

FIG. 2 shows a schematic representation of a cas9-Rep (virus replication associated protein) fusion protein (SEQ ID NO: 14) and an exo1-MS2 fusion protein showing its binding to an aptamer loop (SEQ ID NO: 19). Also shown is an electrophoresis gel demonstrating the activity of the cas9-Rep fusion protein, and examples of other nucleases fused to Rep gene.

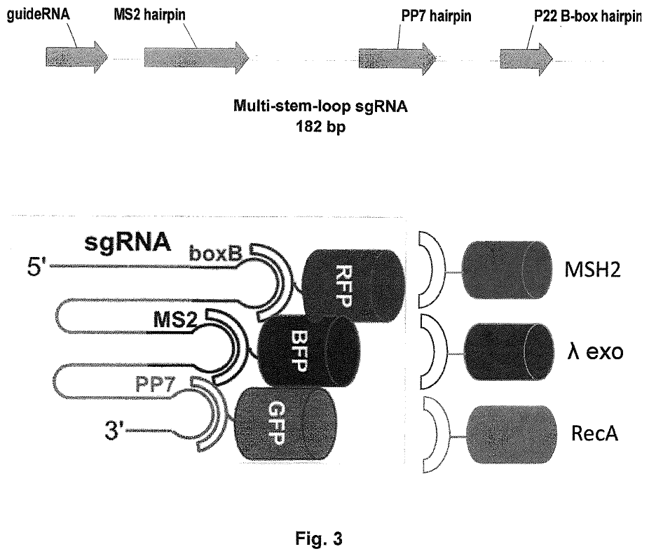

FIG. 3 shows the design of a multi stem-loop sgRNA with hairpins from different bacteriophages (MS2, PP7 and P22 bacteriophages; SEQ ID NO: 19-21).

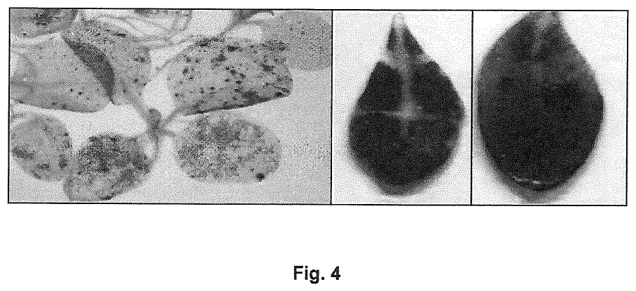

FIG. 4 shows examples of tobacco leaves in which a uidA with a two-base pair frame shift mutation (SEQ ID NO: 2) is repaired. The transgenic lines were assayed for GUS activities as described by McCabe et al., (Nature Biotechnology, 1988, 6, 923-926). Blue colour indicates repair of the mutated uidA gene; the extent of the blue colour indicates the extent of the repair.

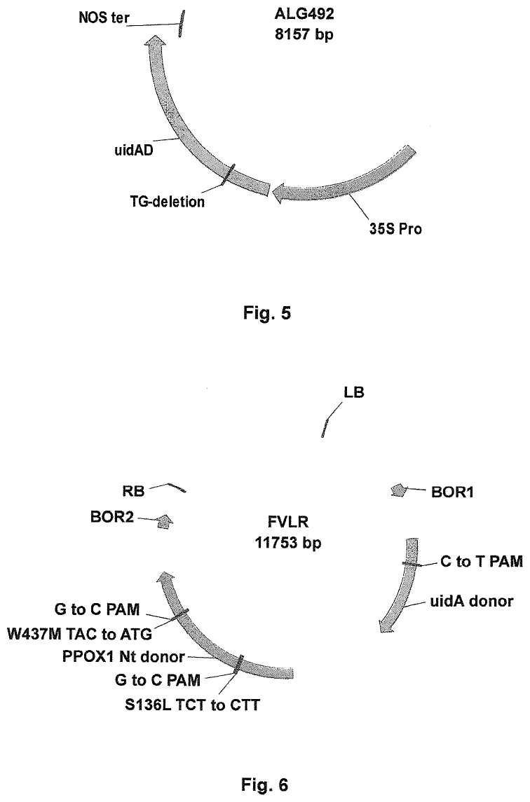

FIG. 5 shows a vector containing mutated uidA gene (SEQ ID NO: 2) from E. coli under the 35S promoter (SEQ ID NO: 1) used for stable transformation of tobacco and subsequent utilisation of stably transformed tobacco lines for gene editing.

FIG. 6 shows a vector for delivery of donor molecules for repair of uidA (SEQ ID NO: 4) in tobacco ALG492 stable transgenic lines, and mutagenesis of PPOX1 gene from tobacco to induce herbicide resistance. BOR1 and BOR2 denote viral origins of replication from beet top curly virus (BCTV) (SEQ ID NO: 7). ‘uidA donor’ contains sequence for repair of a 2 bp deletion with modification in a PAM triplet. ‘PPOX1 Nt donor’ denotes a sequence for generation of mutations in endogenous tobacco PPOX1 gene (SEQ ID NO: 5) to induce herbicide resistance to oxyfluorfen or butafenacil herbicides. Two mutations were designed to introduce herbicide resistance: S136L and W437M. PAM triplets were modified to prevent the donor from being cut by cas9.

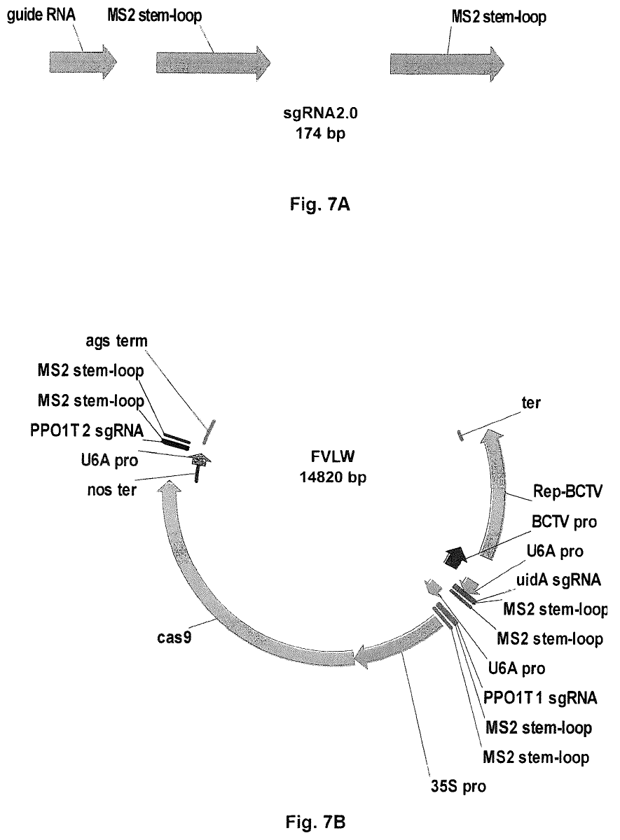

FIG. 7A shows the design of sgRNA for precise targeting of proteins to the double-stranded DNA break. Two stem-loops from bacteriophage MS2 (SEQ ID NO: 19) were introduced into the sgRNA.

FIG. 7B shows the FVLW vector containing cas9 (SEQ ID NO: 8) and Rep-BCTV (SEQ ID NO: 7) cassettes for expression in plants. The construct also has sgRNAs with guide for the uidA gene (SEQ ID NO: 10) and two additional sgRNAs with guides for targeting the tobacco PPOX1 gene (SEQ ID NOs: 10 and 11). The Arabidopsis U6A promoter (SEQ ID NO: 9) was used for expression of sgRNAs containing a gene-specific guide and sgRNAs with two MS2 loops (SEQ ID NO: 19).

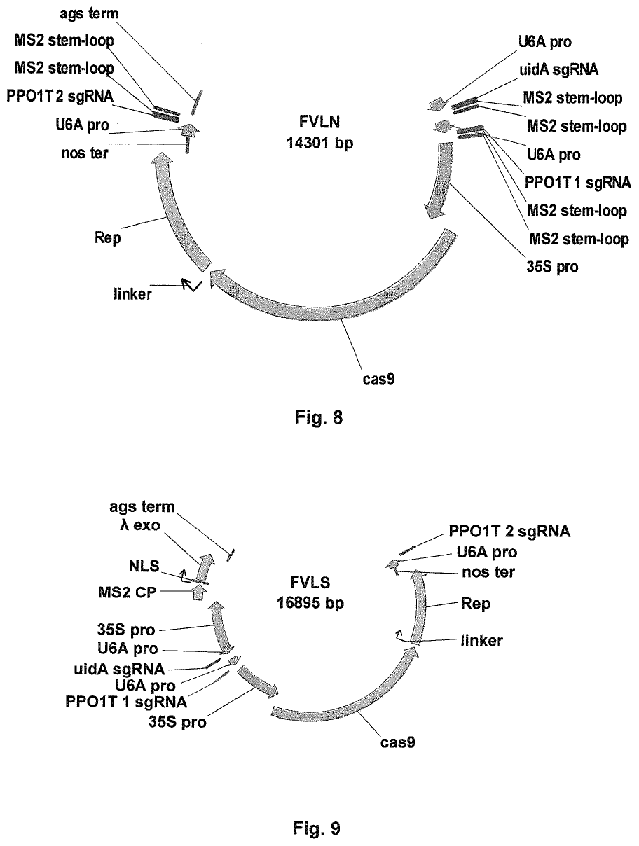

FIG. 8 shows the FVLN vector containing a cas9 gene (SEQ ID NO: 8) translationally fused to Rep-BCTV (SEQ ID NO: 7) yielding a cas9-B-rep fusion gene (SEQ ID NO: 14). The construct also has three sgRNAs with guides for uidA and tobacco PPOX1 genes (SEQ ID NOs: 9-11). The Arabidopsis U6A promoter (SEQ ID NO: 9) was used for expression of sgRNAs containing a gene-specific guide and sgRNAs with two MS2 loops (SEQ ID NO: 19).

FIG. 9 shows the FLVS vector containing a cassette with a cas9 gene (SEQ ID NO: 8) translationally fused to Rep-BCTV (SEQ ID NO: 7) yielding a cas9-B-rep fusion gene (SEQ ID NO: 14), and cassette with the MS2-exol fusion protein (SEQ ID NOs 19 and 15). The construct also has three sgRNAs with guides for uidA and tobacco PPOX1 genes (SEQ ID NOs: 9-11). The Arabidopsis U6A promoter (SEQ ID NO: 9) was used for expression of the sgRNAs containing gene-specific guides and sgRNAs with two MS2 loops (SEQ ID NO: 19).

FIG. 10 shows a selection donor vector for introduction of modifications into the desirable genome locus (SEQ ID NO: 29).

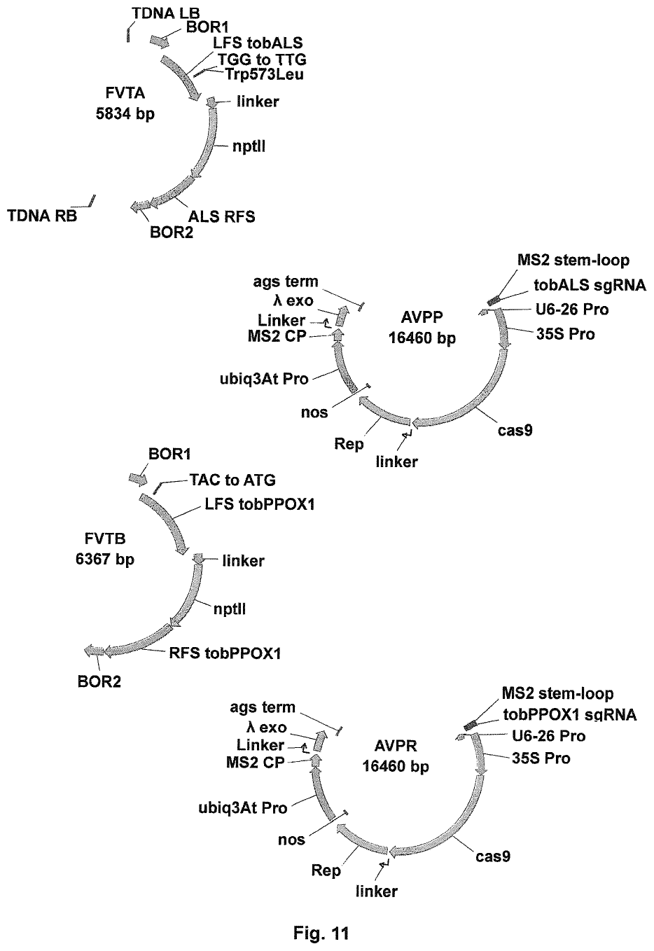

FIG. 11 shows vectors for insertion of nptII gene into tobacco ALS and PPOX1 loci (example 2).

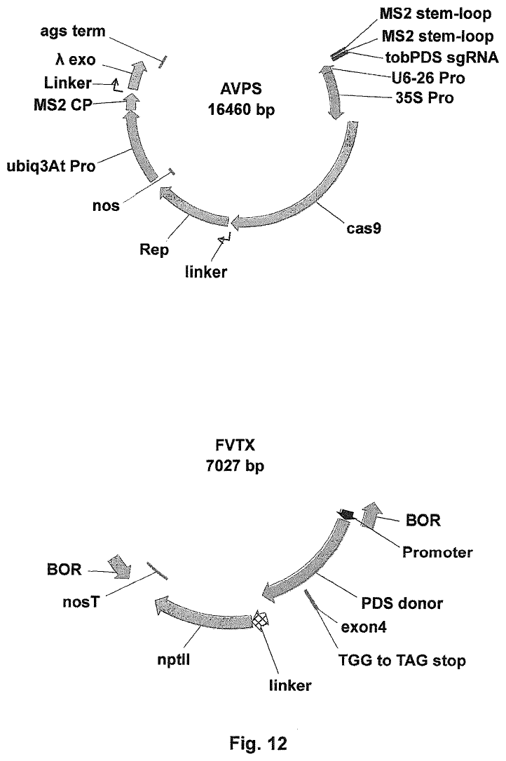

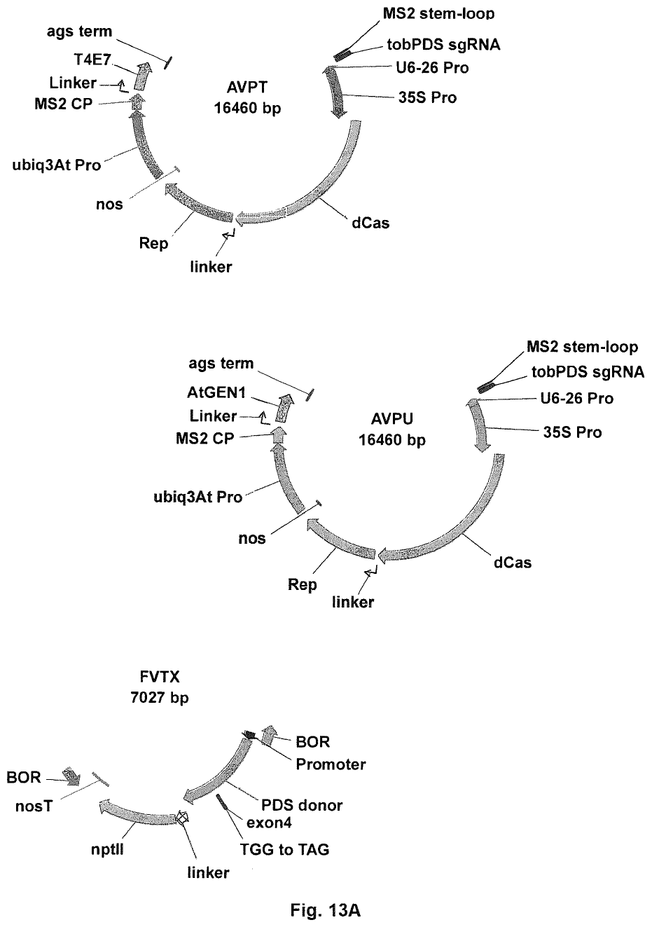

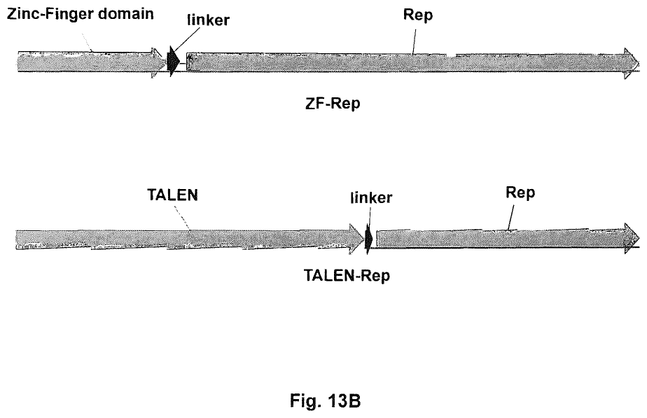

FIG. 12 shows vectors for introduction of knock out mutation in tobacco PDS gene (example 3).

FIG. 13A shows vectors for generation of knock out mutant in tobacco PDS gene using dead Cas9 in combination with Holliday junction resolvases (example 4).

FIG. 13B examples of other DNA-binding domains fused to Rep gene.

### Example 1 Insertion of 2 bp Into the Gene of Tobacco Genome

To assess efficiency of gene targeting in tobacco a set of constructs was prepared for targeting an exogenous uidA gene from E. coli.

Transformations were carried out by infiltration (see method below).

A two base pair deletion was introduced in the uidA gene from E. coli (SEQ ID NO: 2). The modified uidA gene was introduced into tobacco under the cauliflower mosaic virus (CMV) 35S promoter (SEQ ID NO: 1) with a nos terminator (SEQ ID NO: 3) (ALG 492, FIG. 5). The transgenic lines were assayed for GUS activities as described by McCabe et al., (Nature Biotechnology, 1988, 6, 923-926). No GUS activity was detected in transgenic lines due to the frame shift in the uidA gene open reading frame.

The tobacco plants carrying the mutated uidA gene were then co-transformed with a repair donor comprising SEQ ID NO: 4 as part of construct FVLR (FIG. 6) and a construct expressing (i) Cas9 (construct FVLW; FIG. 7b), (ii) Cas9-Rep fusion (construct FVLN; FIG. 8) (SEQ ID NO: 14) or (iii) Cas9-Rep fusion (construct FVLS; FIG. 9) (SEQ ID NO: 14) and an exo1-MS2 fusion protein designed to bind to the sgRNA that is in turn bound to the cas9. The plants subsequently generated were assessed for GUS activity Blue sectors colour confirms repair of the uidA gene in planta using our gene targeting system (FIG. 4). Partial blue colour indicates a transformation yielding a chimeric plant. A fully blue colour indicates a fully or substantially fully transformed plant. The results of these experiments are set out in Table 1.

TABLE 1

| | | | GT efficiency/ |
|---|---|---|---|
| Experiment | Chimeric plants | Blue plants | blue |
| (i) Donor BOR12 - PAM* | 2 out of 35 | 4 out of 35 | 11% |
| uidA sgRNA2.0 | | | |
| 35S-cas9-nos ter// PBCTV- | | | |
| Brep-ter | | | |
| (ii) Donor BOR12 - PAM* | 7 out of 59 | 19 out of 59 | 32% |
| uidA sgRNA2.0 | | | |
| 35S-cas9-Brep-nos ter | | | |
| (iii) Donor BOR12 - PAM* | 7 out of 59 | 29 out of 59 | 49% |
| uidA sgRNA2.0 | | | |
| 35S-cas9-Brep-nos ter// | | | |
| 35S-MS2CP-Exol-ags ter | | | |

These results demonstrate that gene editing mediated by the Cas9-Rep fusion (experiment (ii)) is significantly more efficient than for the control experiment using cas9 alone.

These results also demonstrate that gene editing mediated by the Cas9-Rep fusion and an exo1-MS2 fusion protein designed to bind to the Cas9-sgRNA complex (experiment (iii)) is yet more efficient than either the control experiment using cas9 alone or experiment (ii) using the Cas9-Rep fusion alone.

### Example 2. Insertion of DNA Fragments Into Desirable Locus of Tobacco Genome

Insertion of long DNA sequences into the double-stranded breaks represents a challenge for modification of genomes for different organisms. Here we present an improvement of the insertion efficiency into tobacco genome using a combination of molecules from the invention.

Two targets were chosen for the experiment, namely acetolactate synthase (ALS) and protoporphyrinogen oxydase 1 (PPOX1) genes. Two vectors were designed for insertion at the end of the ALS (SEQ ID NO: 30) and PPOX1 (SEQ ID NO: 31) genes using translational fusion of nptII gene (SEQ ID NO: 32) (FIG. 11). The left flanking sequences (LRF) were modified with specific mutation to generate chlorsulfuron herbicide resistance for ALS and oxyfluorfen herbicide resistance for PPOX1 genes and were translationally fused to nptII gene. The right flanking sequences (RFS) were represented with non-coding regions of the loci. The sgRNA was design for both ALS gene (SEQ ID NO: 33) and PPOX1 genes (SEQ ID NO: 34).

Tobacco plants were transformed using Agrobacterium-mediated method with constructs FTTA and AVPP for insertion in ALS locus, and FTTB and AVPR for PPOX1 locus (FIG. 11). Ten lines were generated for each transformation on kanamycin selection medium, and the generated lines were treated with corresponding herbicides. Two plants out of ten were resistant to chlorsulfuron (ALS locus) and 4 lines out of ten to oxyfluorfen (PPOX1 locus). PCR and sequencing analyses have confirmed insertion of nptII gene into designed loci. Thus, the invention allows introducing both mutation and insertion at the same time.

### Example 3. Generation of Knock Out Mutants in Tobacco Genome

CRISPR/cas9 system is widely used for generation of knock out mutations. However such mutations cannot be controllable as non-homologous end joining (NHEJ) will cause various insertions or deletions of nucleotides at the break site. Such insertion or deletion events are known as ‘indels’.

The invention provides a method for generation of stop codon in the desirable target and selection for the cells/clones/plants using selection donor vector. The selection donor vector can be also designed for both precise deletion and insertion to introduce knock out mutation, if necessary.

A tobacco phytoens desaturase (PDS) gene was chosen for introduction of premature stop codon into one of the gene exons, to cause regeneration of albino plants, as shown by Wang et al., 2010 (doi: 10.1018/j.envexpbot.2009.09.007).

The donor DNA fragment was designed with premature stop codon in the PDS exon (SEQ ID NO: 35) and introduced into the selection donor vector in translational frame with nptII gene (FIG. 10). The corresponding sgRNA was designed for generation of DSB in the locus (SEQ ID NO: 36).

Agrobacterium-mediated transformation of tobacco was performed with constructs FVTX and AVPS (FIG. 12), and plants were regenerated on kanamycin supplemented medium.

### Example 4. Generation of Knock Out Mutations in Tobacco Genome Without Introduction of Double-Stranded DNA Breaks Using the Selection Donor Vector and Holliday Junction Resolvases

We prepared mutated version of cas9-Rep (SEQ ID NO: 37), where both nuclease activities sites were eliminated resulting in so-called dead cas9 nuclease (dCas9-Rep). Although nuclease activities were eliminated, dCas9-Rep still binds to sgRNA and recognises the target. As Rep gene is fused to dCas9, the donor DNA molecule covalently linked to Rep is still tethered to the target and can be annealing with target forming Holliday junctions. Such annealing of donor DNA with target and formation of Holliday junction are suppressed by endogenous helicases. In order to facilitate rapid resolution of Holliday junctions at the target site after annealing of donor DNA, we have co-delivered AVPT vector with resolvase from bacteriophage T4 (T4 exonuclease VII (T4E7)) or AVPU vector with Arabidopsis AtGEN1 resolvase fused to MS2 coat protein to tether it to target site using MS2 stem-loops integrated into sgRNA (FIG. 13). The selection donor vector FVTX was designed to introduce stop codon to tobacco PDS gene as described in example 3. Both bacteriophage and Arabidopsis resolvases have facilitated recovery of mutated plants in combination with selection donor vector.

### Transformation Method-Infiltration

### Transformation of Tobacco Leaf Explants With Agrobacterium Strain AGLI

All items are autoclave-sterilised prior to use. Filter sterilize antibiotics to prevent fungal growth, keep antibiotics for plant tissue culture in separate box.

Sterilize plant material: take plants of about 9 cm high which have not started to flower. Cut leaves having a cuticle (4-6 leaves per construct, enough to cut 100 explants), dip in 70% Ethanol and immediately dip in 1% Na-hypochlorite (use a bottle of bleach that is <3 months old because the chlorine gas evaporates), hold leaves with forceps and stir in it for 20 min. Avoid damaging the cuticle otherwise bleach will enter the vascular system. Rinse briefly in sterile water 5-6 times and leave in water until ready to be cut.

Co-cultivation of Agrobacterium with tobacco explants: grow AGLI in LB or L broth with appropriate antibiotics overnight at 28-30° C., the next day re-suspend Agrobacterium in co-cultivation solution so that the final concentration has an OD₆ₒₒₙₘ of around 0.4-0.6. Place tobacco leaves in co-culture broth and cut squares of 1-1.5 cm×1-1.5 cm with a rounded sterile scalpel using a rolling action. Dip the leaf explants in the Agrobacterium solution with sterile forceps (stored in 100% ethanol, flamed and let to cool prior to touching the leaf tissue) blot on sterile Whatman paper and transfer on non-selective TSM plates (6 explants per plate; need to prepare about 15 plates per construct). Repeat this procedure for each construct, making sure that the scalpel and forceps are dipped in ethanol and flamed between each construct to prevent cross-contamination. Leave for 2 days only for AGLI (3-4 days for other Agrobacterium strains).

Transfer on selective TSM plates: use sterile flamed forceps to pick up and wash explants in 100 ml co-cultivation broth supplemented with Timentin 320 mg/l (one aliquot per construct), shake well, blot on sterile Whatman paper and place the washed explants on selective TSM plates supplemented with appropriate selective antibiotics and Timentin 320 mg/l to kill Agrobacterium.

Shoot regeneration: takes around 1 month to see shoots appear, explants should be transferred onto fresh plates every 10-14 days. Watch out for AGLI recurrent growth, if Timentin is not enough to kill Agrobacterium, add cefotaxime at 250 mg/l.

Root regeneration: Takes around 1 week. Shoots are cut from the explants and place in growth boxes containing TRM supplemented with the appropriate selective antibiotics and Timentin 320 mg/l+cefotaxime 250 mg/l to prevent Agrobacterium recurrent growth. Maintain plants in TRM boxes: sub them every two weeks until ready to be transferred into a glasshouse.

Adaptation to glasshouse conditions: soak peat pellets in sterile water until they swell to normal size and carefully plant one plant per pellet, incubate the plants under 100% humidity conditions in a propagator, gradually opening the little windows until plants adapt to normal atmosphere over several days.

Assay the transgenic plants for GUS activities as described by McCabe et al., (Nature Biotechnology, 1988, 6, 923-926).

### Reagents:

Co-culture: MS with vitamins and MES+0.1 mg/1 NAA+1 mg/1 BA+3% sucrose, pH 5.7
TSM: MS with vitamins and MES+0.1 mg/1 NAA+1 mg/1 BA+3% sucrose, pH 5.7, 0.2% gelrite
TRM: \hat{ } MS salts with vitamins and MES+0.5% sucrose, pH 5.7, 0.2% gelrite. Autoclave.

### Antibiotic Concentrations for Agrobacterium LB or L Cultures:

To grow AGLI carrying pGreen/pSOUP: Carbenicillin 100 mg/1, Tetracycline 5 mg/ml, Rifampicin 50 mg/ml, Kanamycin 50 mg/ml AGLI carrying pSOUP: Carbenicilin 100 mg/1, Tetracycline 5 mg/ml, Rifampicin 50 mg/ml. AGLI empty: Carbenicillin 100 mg/1, Rifampicin 50 mg/ml.

### Antibiotic Concentrations for Plant Culture:

Kanamycin: 300 mg/l (100 mg/1 if using benthamiana) Hygromycin: 30 mg/l (10 mg/1 if using benthamiana) PPT: 20 mg/l (2 mg/l if using benthamiana)
Timentin: 320 mg/l. It is used to kill agrobacterium, fairly-unstable make up small amount of stock, store in freezer for up to 1 month (after that the antibiotic is no longer efficient). Cefotaxime: 250 mg/l. Also used to kill agrobacterium, add to TS.

### Nucleotide Sequences

35S promoter
(SEQ ID NO: 1)
ggaattccaatcccacaaaaatctgagcttaacagcacagttgct
cctctcagagcagaatcgggtattcaacaccctcatatcaactac
tacgttgtgtataacggtccacatgccggtatatacgatgactgg
ggttgtacaaaggcggcaacaaacggcgttcccggagttgcacac
aagaaatttgccactattacagaggcaagagcagcagctgacgcg
tacacaacaagtcagcaaacagacaggttgaacttcatccccaaa
ggagaagctcaactcaagcccaagagctttgctaaggccctaaca
agcccaccaaagcaaaaagcccactggctcacgctaggaaccaaa
aggcccagcagtgatccagccccaaaagagatctcctttgccccg
gagattacaatggacgatttcctctatctttacgatctaggaagg
aagttcgaaggtgaagtagacgacactatgttcaccactgataat
gagaaggttagcctcttcaatttcagaaagaatgctgacccacag
atggttagagaggcctacgcagcaggtctcatcaagacgatctac
ccgagtaacaatctccaggagatcaaataccttcccaagaaggtt
aaagatgcagtcaaaagattcaggactaattgcatcaagaacaca
gagaaagacatatttctcaagatcagaagtactattccagtatgg
acgattcaaggcttgcttcataaaccaaggcaagtaatagagatt
ggagtctctaaaaaggtagttcctactgaatctaaggccatgcat
ggagtctaagattcaaatcgaggatctaacagaactcgccgtgaa
gactggcgaacagttcatacagagtcttttacgactcaatgacaa
gaagaaaatcttcgtcaacatggtggagcacgacactctggtcta
ctccaaaaatgtcaaagatacagtctcagaagaccaaagggctat
tgagacttttcaacaaaggataatttcgggaaacctcctcggatt
ccattgcccagctatctgtcacttcatcgaaaggacagtagaaaa
ggaaggtggctcctacaaatgccatcattgcgataaaggaaaggc
tatcattcaagatctctctgccgacagtggtcccaaagatggacc
cccacccacgaggagcatcgtggaaaaagaagacgttccaaccac
gtcttcaaagcaagtggattgatgtgacatctccactgacgtaag
ggatgacgcacaatcccactatccttcgcaagacccttcctctat
ataaggaagttcatttcatttggagaggacacgctcgagtataag
agctcatttttacaacaattaccaacaacaacaaacaacaaacaa
cattacaattacatttacaattatcgata
uidA gene with 2bp deletion
(SEQ ID NO: 2)
atgttacgtcctgtagaaaccccaacccgtgaaatcaaaaaactc
gacggcctgtgggcattcagtctggatcgcgaaaactgtggaatt
gatcagcgttggtgggaaagcgcgttacaagaaagccgggcaatt
gctgtgccaggcagttttaacgatcagttcgccgatgcagatatt
cgtaattatgcgggcaacgtctggtatcagcgcgaagtctttata
ccgaaaggttgggcaggccagcgtatcgtgctgcgtttcgatgcg
gtcactcattacggcaaagtgtgggtcaataatcaggaagtgatg
gagcatcagggcggctatacgccatttgaagccgatgtcacgccg
tatgttattgccgggaaaagtacgtatcaccgtttgtgtgaacaa
cgaactgaactggcagactatcccgccgggaatggtgattaccga
cgaaaacggcaagaaaaagcagtcttacttccatgatttctttaa
ctatgccgcgatccatcgcagcgtaatgctctacaccacgccgaa
cacctgggtggacgatatcaccgtggtgacgcatgtcgcgcaaga
ctgtaaccacgcgtctgttgactggcaggtggtggccaatggtga
tgtcagcgttgaactgcgtgatgcggatcaacaggtggttgcaac
tggacaaggcactagcgggactttgcaagtggtgaatccgcacct
ctggcaaccgggtgaaggttatctctatgaactgtgcgtcacagc
caaaagccagacagagtgtgatatctacccgcttcgcgtcggcat
ccggtcagtggcagtgaagggcgaacagttcctgattaaccacaa
accgttctactttactggctttggtcgtcatgaagatgcggactt
acgtggcaaaggattcgataacgtgctgatggtgcacgaccacgc
attaatggactggattggggccaactcctaccgtacctcgcatta
cccttacgctgaagagatgctcgactgggcagatgaacatggcat
cgtggtgattgatgaaactgctgctgtcggctttaacctctcttt
aggcattggtttcgaagcgggcaacaagccgaaagaactgtacag
cgaagaggcagtcaacggggaaactcagcaagcgcacttacaggc
gattaaagagctgatagcgcgtgacaaaaaccacccaagcgtggt
gatgtggagtattgccaacgaaccggatacccgtccgcaagtgca
cgggaatatttcgccactggcggaagcaacgcgtaaactcgaccc
gacgcgtccgatcacctgcgtcaatgtaatgttctgcgacgctca
caccgataccatcagcgatctctttgatgtgctgtgcctgaaccg
ttattacggatggtatgtccaaagcggcgatttggaaacggcaga
gaaggtactggaaaaagaacttctggcctggcaggagaaactgca
tcagccgattatcatcaccgaatacggcgtggatacgttagccgg
gctgcactcaatgtacaccgacatgtggagtgaagagtatcagtg
tgcatggctggatatgtatcaccgcgtctttgatcgcgtcagcgc
cgtcgtcggtgaacaggtatggaatttcgccgattttgcgacctc
gcaaggcatattgcgcgttggcggtaacaagaaagggatcttcac
tcgcgaccgcaaaccgaagtcggcggcttttctgctgcaaaaacg
ctggactggcatgaacttcggtgaaaaaccgcagcagggaggcaa
acaatga
nos terminator
(SEQ ID NO: 3)
gtcgaagcagatcgttcaaacatttggcaataaagtttcttaaga
ttgaatcctgttgccggtcttgcgatgattatcatataatttctg
ttgaattacgttaagcatgtaataattaacatgtaatgcatgacg
ttatttatgagatgggtttttatgattagagtcccgcaattatac
atttaatacgcgatagaaaacaaaatatagcgcgcaaactaggat
aaattatcgcgcgcggtgtcatctatgttactagatcgac
uidA donor
(SEQ ID NO: 4)
cgtcctgtagaaaccccaacccgtgaaatcaaaaaactcgacggc
ctgtgggcattcagtctggatcgcgaaaactgtggaattgatcag
cgttggtgggaaagcgcgttacaagaaagccgggcaattgctgtg
ccaggcagttttaacgatcagttcgccgatgcagatattcgtaat
tatgcgggcaacgtctggtatcagcgcgaagtctttataccgaaa
ggttgggcaggccagcgtatcgtgctgcgtttcgatgcggtcact
cattacggcaaagtgtgggtcaataatcaggaagtgatggagcat
cagggcggctatacgccatttgaagccgatgtcacgccgtatgtt
attgccgggaaaagtgtacgtatcactgtttgtgtgaacaacgaa
ctgaactggcagactatcccgccgggaatggtgattaccgacgaa
aacggcaagaaaaagcagtcttacttccatgatttctttaactat
gccggaatccatcgcagcgtaatgctctacaccacgccgaacacc
tgggtggacgatatcaccgtggtgacgcatgtcgcgcaagactgt
aaccacgcgtctgttgactggcaggtggtggccaatggtgatgtc
agcgttgaactgcgtgatgcggatcaacaggtggttgcaactgga
caaggcactagcgggactttgcaagtggtgaatccgcacctctgg
caaccgggtgaaggttatctctatgaactgtgcgtcacagccaaa
agccagacagagtgtgatatctacccgcttcgcgtcggcatccgg
tcagtggcagtgaagggcgaacagttcctgattaaccacaaaccg
ttctactttactggctttggtcgtcatgaagatgcggacttgcgt
ggcaaaggattcgataacgtgctgatggtgcacgaccacgcatta
atggactggattggggccaactcctaccgtacctcgcattaccct
tacgctgaagagatgctcgactgggcagatgaacatggcatcgtg
gtgattgatgaaactgctgctgtcggctttaacctctctttaggc
attggtttcgaagcgggcaacaagccgaaagaactgtacagcgaa
gaggcagtcaacggggaaactcagcaagcgcacttacaggcgatt
aaagagctgatagcgcgtgacaaaaaccacccaagcgtggtgatg
tggagtattgccaacgaaccggatacccgtccgcaaggtgcacgg
gaatatttcgcgccactggcggaagcaacgcgtaaactcgacccg
acgcgtccgatcacctgcgtcaatgtaatgttctgcgacgctcac
accgataccatcagcgatctctttgatgtgctgtgcctgaaccgt
tattacggatggtatgtccaaagcggcgatttggaaacggcagag
aaggtactggaaaaagaacttctggcctggcaggagaaactgcat
cagccgattatcatcaccga
tobacco PPOX1 donor
(SEQ ID NO: 5)
tgctgccggatgcaatcagtgcaaggtgcccttctcctttcccag
ttcaatgtctccaccccactcccacccccagtataaaaattcagg
ggggaaaaagagaagaaaaatatcaaaatttaaggactagttaaa
gttcagttttggcattttttctgcctaagtgcaatattatcaaag
gtgaaaagcgcaaaaaagctctaaagtctattggggctttaagcg
caaagcgcaaataaagcatgggctttaatgcagaaaggctcaaat
gaaaatgaaaagaaaaagaaaactacaaatatgtatgtatagtcc
aagactaatatctataagcatgaataccaaatatatggacaaaga
cattgaagaaactttattataaagtgaaatatcaattgtttagca
tcgcctcttcaggattacgcttattggcaaggaaaagtatgcttt
tagagccttgatgacaacgctgaagcgcccgctaagcgaggcaaa
gcgctcaacatgttttgagcctcgcttcagggcttaagcgttatc
aaagtgtgttctttggtgcccttgttcaagcagttctctagtatt
tttaccttggctccaccccactcccaccccctgtataaacgttta
gggggggaaaagaaagagaagaaaaattatcaaaagttaagaaag
ctaagttttggcttttttctgctaggtgtgttctttggtgctctt
gttcaagcagttctctagtatttttaccttggaaatgctaataag
ttgtcgccgtgtttccttagattgggaagcaaattaaaactatca
tggaagcttcttagcattactaagtcagaaaaagcaggatatcgc
ttgacatacgagacaccagaaggagtagtttctcttcaaagtcga
agcattgtcatgactgtgccatcctatgtagcaagcaacatatta
cgtcctctttcggtgtgtttccattactgtttgctccaaagtcgt
tattgctattgttcatttggcactctaatatagaaacttggataa
aaaaggggatcctttaatttttctttttcatttggactaggaaaa
gcggctcaaacacatgtataacagtcatatactgtgtataatatg
tgtatgcaatataatgtgcagagaaagacttatcggagagaagag
aattaaaaaaaagaacatttgtgtcacacggagaattaacatgta
catgcttaatgaggtcaagagcgcaaccgtatcccacccctcatg
aaagattgaacctgtatctcatgttattgtgagcttttttcatat
ataaatttatgagcaattttctttaaatgctttgaccttggattt
ttcgaaggttactacttctgataattgctttgcaagaaatgcggc
tcacttaaaatgggaaacgtttatctataggttgtggttcagctt
cttctgtttcttgctaaagcaagatgtctcctaaacttatattaa
actcaggttgccgcagcagatgcactttcaaatttctactatccc
ccagttggagcagtcacaatttcatatcctcaagaagctattcgt
gatgaacgtctggttgatggtgaactaaagggatttgggcagttg
catccacgtacacagggagtggaaacactaggtagctttctgttt
gaaggattcactctaattttaacttcaagaatccatattgaagat
ggaaaacttttattacgacaattgcaagatgcaactcactgaagt
agcattcctctattgcatctgcaggaataatatatagcgtatcag
tcttccctgaccatcattacctagaaattttgacagtaccatgca
aatcgaagatgaatgagttcagaacaataacatcaggatttgatt
catcttaaatataatacttgcgtttcatatgcaggaacgatatat
agttcatcactcttccctaaccgtgccccaaaaggtcgggtgcta
ctcttgaacatgattgcaggagcaaaaaatccggaaattttgtct
aaggtaaggcatttaaattgagcgcaacttcaagtcctgccggta
ttgatttatcataatgaatattcatgggctggccagttggggcat
cgcttgaaggttcctatggtaacaaacagaaaactgagaatgacc
ttatcccctgatctcttttggttgcaaatacgttaaccttgcagt
gatgaggagaatgttttcagtgttattttctctagattaatggcg
tagttggtttggattacaaaaccgtttaggcttatatgtgattag
agtctccaggtttttgatctagcagtaagagcaaaatgtgtgatg
ttttaaaaaaacagtcaagtgattacaaatactcagaatagtttc
accataaagaactaaagagctaagaagattgtggtttctctttcc
tctatgttcgactgcattttgtgggttttccatagcatgactttc
ttttatggtacttgtacgaaagcatttcgctgttattctttatgc
ttcagggcctagaataaattgctttgagatttgatccctcattct
tcttgcagacggagagccaacttgtggaagtagttgatcgtgacc
tcagaaaaatgcttatagaacccaaagcacaagatccccttgttg
tgggtgtgcgagtatggccac
viral origin of replication (BOR) from Beet
Curly Top Virus (BCTV)
(SEQ ID NO: 6)
tcctgtactccgatgacgtggcttagcatattaacatatctattg
gagtattggagtattatatatattagtacaactttcataagggcc
atccgttataatattaccggatggcccgaaaaaaatgggcaccca
atcaaaacgtgacacgtggaaggggactgttgaatgatgtgacgt
ttttgagcgggaaacttcctgaag
BCTV rep gene
(SEQ ID NO: 7)
atgcctcctactaaaagatttcgtattcaagcaaaaaacatattt
cttacatatcctcagtgttctctttcaaaagaagaagctcttgag
caaattcaaagaatacaactttcatctaataaaaaatatattaaa
attgccagagagctacacgaagatgggcaacctcatctccacgtc
ctgcttcaactcgaaggaaaagttcagatcacaaatatcagatta
ttcgacctggtatccccaaccaggtcagcacatttccatccaaac
attcagagagctaaatccagctccgacgtcaagtcctacgtagac
aaggacggagacacaattgaatggggagaattccagatcgacggt
agaagtgctagaggaggtcaacagacagctaacgactcatatgcc
aaggcgttaaacgcaacttctcttgaccaagcacttcaaatattg
aaggaagaacaaccaaaggattacttccttcaacatcacaatctt
ttgaacaatgctcaaaagatatttcagaggccacctgatccatgg
actccactatttcctctgtcctcattcacaaacgttcctgaggaa
atgcaagaatgggctgatgcatatttcggggttgatgccgctgcg
cggcctttaagatataatagtatcatagtagagggtgattcaaga
acagggaagactatgtgggctagatctttaggggcccacaattac
atcacagggcacttagattttagccctagaacgtattatgatgaa
gtggaatacaacgtcattgatgacgtagatcccacttacttaaag
atgaaacactggaaacaccttattggagcacaaaaggagtggcag
acaaacttaaagtatggaaaaccacgtgtcattaaaggtggtatc
ccctgcattatattatgcaatccaggacctgagagctcataccaa
caatttcttgaaaaaccagaaaatgaagcccttaagtcctggaca
ttacataattcaaccttctgcaaactccaaggtccgctctttaat
aaccaagcagcagcatcctcgcaaggtgactctaccctgtaactg
ccacttcacaatacaccatgaatgtaatagaggattttcgcacag
gggaacctattactctccatcaggcaacaaattccgtcgaattcg
agaatgtaccgaatccactgtatatgaaactcctatggttcgaga
gatacgggccaatctatcaactgaagatacaaatcagattcaact
acaacctccggagagcgttgaatcttcacaagtgctggatagagc
tgacgataactggatcgaacaggatattgactggaccccgtttct
tgaaggtcttgaaaaagagactagagatatacttggataatctag
gtttaatttgtattaataatgtaattagaggtttaaatcatgtcc
tgtatgaagaatttacttttgtatcaagtgtaattcagaaccaga
gtgttgcaatgaacttgtactaatttcattattaataataaatat
tattaataaaaatagcatctacaattgccaaataatgtggcatac
atattagtattatccgtattatcattaacaacaacatagagtaaa
gcattctccttcacgtcttcatacttccc
cas9 gene
(SEQ ID NO: 8)
atggacaagaagtacagcatcggcctggacatcggcacgaactcg
gtgggctgggcggtgatcacggacgagtacaaggtgccctccaag
aagttcaaggtgctgggcaacaccgaccgccactcgatcaagaag
aacctgatcggcgccctgctgttcgactccggcgagaccgccgag
gcgacgcgcctgaagcgcaccgcgcgtcgccgctacacgcgtcgc
aagaaccgcatctgctacctgcaggagatcttcagcaacgagatg
gccaaggtggacgactcgttcttccaccgcctggaggagtccttc
ctggtggaggaagacaagaagcacgagcgccaccccatcttcggc
aacatcgtggacgaggtggcctaccacgagaagtacccgacgatc
taccacctgcgcaagaagctggtggacagcaccgacaaggcggac
ctgcgcctgatctacctggccctggcgcacatgatcaagttccgc
ggccacttcctgatcgagggcgacctgaaccccgacaactcggac
gtggacaagctgttcatccagctggtgcagacctacaaccagctg
ttcgaggagaacccgatcaacgcctccggcgtggacgccaaggcg
atcctgagcgcgcgcctgtccaagagccgtcgcctggagaacctg
atcgcccagctgcccggcgagaagaagaacggcctgttcggcaac
ctgatcgcgctgtcgctgggcctgacgccgaacttcaagtccaac
ttcgacctggccgaggacgcgaagctgcagctgagcaaggacacc
tacgacgacgacctggacaacctgctggcccagatcggcgaccag
tacgcggacctgttcctggccgcgaagaacctgtcggacgccatc
ctgctgtccgacatcctgcgcgtgaacaccgagatcacgaaggcc
cccctgtcggcgtccatgatcaagcgctacgacgagcaccaccag
gacctgaccctgctgaaggcgctggtgcgccagcagctgccggag
aagtacaaggagatcttcttcgaccagagcaagaacggctacgcc
ggctacatcgacggcggcgcgtcgcaagaggagttctacaagttc
atcaagcccatcctggagaagatggacggcacggaggagctgctg
gtgaagctgaaccgcgaggacctgctgcgcaagcagcgcaccttc
gacaacggcagcatcccccaccagatccacctgggcgagctgcac
gccatcctgcgtcgccaagaggacttctacccgttcctgaaggac
aaccgcgagaagatcgagaagatcctgacgttccgcatcccctac
tacgtgggcccgctggcccgcggcaacagccgcttcgcgtggatg
acccgcaagtcggaggagaccatcacgccctggaacttcgaggaa
gtggtggacaagggcgccagcgcgcagtcgttcatcgagcgcatg
accaacttcgacaagaacctgcccaacgagaaggtgctgccgaag
cactccctgctgtacgagtacttcaccgtgtacaacgagctgacg
aaggtgaagtacgtgaccgagggcatgcgcaagcccgccttcctg
agcggcgagcagaagaaggcgatcgtggacctgctgttcaagacc
aaccgcaaggtgacggtgaagcagctgaaagaggactacttcaag
aagatcgagtgcttcgacagcgtggagatctcgggcgtggaggac
cgcttcaacgccagcctgggcacctaccacgacctgctgaagatc
atcaaggacaaggacttcctggacaacgaggagaacgaggacatc
ctggaggacatcgtgctgaccctgacgctgttcgaggaccgcgag
atgatcgaggagcgcctgaagacgtacgcccacctgttcgacgac
aaggtgatgaagcagctgaagcgtcgccgctacaccggctggggc
cgcctgagccgcaagctgatcaacggcatccgcgacaagcagtcc
ggcaagaccatcctggacttcctgaagagcgacggcttcgcgaac
cgcaacttcatgcagctgatccacgacgactcgctgaccttcaaa
gaggacatccagaaggcccaggtgtcgggccagggcgactccctg
cacgagcacatcgccaacctggcgggctcccccgcgatcaagaag
ggcatcctgcagaccgtgaaggtggtggacgagctggtgaaggtg
atgggccgccacaagccggagaacatcgtgatcgagatggcccgc
gagaaccagaccacgcagaagggccagaagaacagccgcgagcgc
atgaagcgcatcgaggaaggcatcaaggagctgggctcgcagatc
ctgaaggagcaccccgtggagaacacccagctgcagaacgagaag
ctgtacctgtactacctgcagaacggccgcgacatgtacgtggac
caggagctggacatcaaccgcctgtccgactacgacgtggaccac
atcgtgccccagagcttcctgaaggacgactcgatcgacaacaag
gtgctgacccgcagcgacaagaaccgcggcaagagcgacaacgtg
ccgtcggaggaagtggtgaagaagatgaagaactactggcgccag
ctgctgaacgccaagctgatcacgcagcgcaagttcgacaacctg
accaaggccgagcgcggtggcctgtcggagctggacaaggcgggc
ttcatcaagcgccagctggtggagacccgccagatcacgaagcac
gtggcgcagatcctggactcccgcatgaacacgaagtacgacgag
aacgacaagctgatccgcgaggtgaaggtgatcaccctgaagtcc
aagctggtcagcgacttccgcaaggacttccagttctacaaggtg
cgcgagatcaacaactaccaccacgcccacgacgcgtacctgaac
gccgtggtgggcaccgcgctgatcaagaagtaccccaagctggag
agcgagttcgtgtacggcgactacaaggtgtacgacgtgcgcaag
atgatcgccaagtcggagcaggagatcggcaaggccaccgcgaag
tacttcttctactccaacatcatgaacttcttcaagaccgagatc
acgctggccaacggcgagatccgcaagcgcccgctgatcgagacc
aacggcgagacgggcgagatcgtgtgggacaagggccgcgacttc
gcgaccgtgcgcaaggtgctgagcatgccccaggtgaacatcgtg
aagaagaccgaggtgcagacgggcggcttctccaaggagagcatc
ctgccgaagcgcaactcggacaagctgatcgcccgcaagaaggac
tgggaccccaagaagtacggcggcttcgactccccgaccgtggcc
tacagcgtgctggtggtggcgaaggtggagaagggcaagtccaag
aagctgaagagcgtgaaggagctgctgggcatcaccatcatggag
cgcagctcgttcgagaagaaccccatcgacttcctggaggccaag
ggctacaaagaggtgaagaaggacctgatcatcaagctgccgaag
tactcgctgttcgagctggagaacggccgcaagcgcatgctggcc
tccgcgggcgagctgcagaagggcaacgagctggccctgcccagc
aagtacgtgaacttcctgtacctggcgtcccactacgagaagctg
aagggctcgccggaggacaacgagcagaagcagctgttcgtggag
cagcacaagcactacctggacgagatcatcgagcagatctcggag
ttctccaagcgcgtgatcctggccgacgcgaacctggacaaggtg
ctgagcgcctacaacaagcaccgcgacaagcccatccgcgagcag
gcggagaacatcatccacctgttcaccctgacgaacctgggcgcc
ccggccgcgttcaagtacttcgacaccacgatcgaccgcaagcgc
tacacctccacgaaagaggtgctggacgcgaccctgatccaccag
agcatcaccggcctgtacgagacgcgcatcgacctgagccagctg
ggcggcgactcccgcgcggacccgaagaagaagcgcaaggtgtaa
Arabidopsis U6A promoter
(SEQ ID NO: 9)
catcttcattcttaagatatgaagataatcttcaaaaggcccctg
ggaatctgaaagaagagaagcaggcccatttatatgggaaagaac
aatagtatttcttatataggcccatttaagttgaaaacaatcttc
aaaagtcccacatcgcttagataagaaaacgaagctgagtttata
tacagctagagtcgaagtagt
sgRNA2.0 with MS2 aptamer hairpins for uidA gene
(SEQ ID NO: 10)
cagttcgttgttcacacaagttttagagctaggccaacatgagga
tcacccatgtctgcagggcctagcaagttaaaataaggctagtcc
gttatcaacttggccaacatgaggatcacccatgtctgcagggcc
aagtggcaccgagtcggtgcttttttttttt
sgRNA2.0-1 for tobacco PPOX1 gene
(SEQ ID NO: 11)
attactaagtcagaaaagttttagagctaggccaacatgaggatc
acccatgtctgcagggcctagcaagttaaaataaggctagtccgt
tatcaacttggccaacatgaggatcacccatgtctgcagggccaa
gtggcaccgagtcggtgcttttttttttt
sgRNA2.0-2 for tobacco PPOX1 gene
(SEQ ID NO: 12)
tgctactcttgaactacatggttttagagctaggccaacatgagg
atcacccatgtctgcagggcctagcaagttaaaataaggctagtc
cgttatcaacttggccaacatgaggatcacccatgtctgcagggc
caagtggcaccgagtcggtgcttttttttttt
ags terminator
(SEQ ID NO: 13)
gaattaacagaggtggatggacagacccgttcttacaccggactg
ggcgcgggataggatattcagattgggatgggattgagcttaaag
ccggcgctgagaccatgctcaaggtaggcaatgtcctcagcgtcg
agcccggcatctatgtcgagggcattggtggagcgcgcttcgggg
ataccgtgcttgtaactgagaccggatatgaggccctcactccgc
ttgatcttggcaaagatatttgacgcatttattagtatgtgttaa
ttttcatttgcagtgcagtattttctattcgatctttatgtaatt
cgttacaattaataaatattcaaatcagattattgactgtcattt
gtatcaaatcgtgtttaatggatatttttattataatattgatga
t
cas9-B-rep fusion gene
(SEQ ID NO: 14)
atggacaagaagtacagcatcggcctggacatcggcacgaactcg
gtgggctgggcggtgatcacggacgagtacaaggtgccctccaag
aagttcaaggtgctgggcaacaccgaccgccactcgatcaagaag
aacctgatcggcgccctgctgttcgactccggcgagaccgccgag
gcgacgcgcctgaagcgcaccgcgcgtcgccgctacacgcgtcgc
aagaaccgcatctgctacctgcaggagatcttcagcaacgagatg
gccaaggtggacgactcgttcttccaccgcctggaggagtccttc
ctggtggaggaagacaagaagcacgagcgccaccccatcttcggc
aacatcgtggacgaggtggcctaccacgagaagtacccgacgatc
taccacctgcgcaagaagctggtggacagcaccgacaaggcggac
ctgcgcctgatctacctggccctggcgcacatgatcaagttccgc
ggccacttcctgatcgagggcgacctgaaccccgacaactcggac
gtggacaagctgttcatccagctggtgcagacctacaaccagctg
ttcgaggagaacccgatcaacgcctccggcgtggacgccaaggcg
atcctgagcgcgcgcctgtccaagagccgtcgcctggagaacctg
atcgcccagctgcccggcgagaagaagaacggcctgttcggcaac
ctgatcgcgctgtcgctgggcctgacgccgaacttcaagtccaac
ttcgacctggccgaggacgcgaagctgcagctgagcaaggacacc
tacgacgacgacctggacaacctgctggcccagatcggcgaccag
tacgcggacctgttcctggccgcgaagaacctgtcggacgccatc
ctgctgtccgacatcctgcgcgtgaacaccgagatcacgaaggcc
cccctgtcggcgtccatgatcaagcgctacgacgagcaccaccag
gacctgaccctgctgaaggcgctggtgcgccagcagctgccggag
aagtacaaggagatcttcttcgaccagagcaagaacggctacgcc
ggctacatcgacggcggcgcgtcgcaagaggagttctacaagttc
atcaagcccatcctggagaagatggacggcacggaggagctgctg
gtgaagctgaaccgcgaggacctgctgcgcaagcagcgcaccttc
gacaacggcagcatcccccaccagatccacctgggcgagctgcac
gccatcctgcgtcgccaagaggacttctacccgttcctgaaggac
aaccgcgagaagatcgagaagatcctgacgttccgcatcccctac
tacgtgggcccgctggcccgcggcaacagccgcttcgcgtggatg
acccgcaagtcggaggagaccatcacgccctggaacttcgaggaa
gtggtggacaagggcgccagcgcgcagtcgttcatcgagcgcatg
accaacttcgacaagaacctgcccaacgagaaggtgctgccgaag
cactccctgctgtacgagtacttcaccgtgtacaacgagctgacg
aaggtgaagtacgtgaccgagggcatgcgcaagcccgccttcctg
agcggcgagcagaagaaggcgatcgtggacctgctgttcaagacc
aaccgcaaggtgacggtgaagcagctgaaagaggactacttcaag
aagatcgagtgcttcgacagcgtggagatctcgggcgtggaggac
cgcttcaacgccagcctgggcacctaccacgacctgctgaagatc
atcaaggacaaggacttcctggacaacgaggagaacgaggacatc
ctggaggacatcgtgctgaccctgacgctgttcgaggaccgcgag
atgatcgaggagcgcctgaagacgtacgcccacctgttcgacgac
aaggtgatgaagcagctgaagcgtcgccgctacaccggctggggc
cgcctgagccgcaagctgatcaacggcatccgcgacaagcagtcc
ggcaagaccatcctggacttcctgaagagcgacggcttcgcgaac
cgcaacttcatgcagctgatccacgacgactcgctgaccttcaaa
gaggacatccagaaggcccaggtgtcgggccagggcgactccctg
cacgagcacatcgccaacctggcgggctcccccgcgatcaagaag
ggcatcctgcagaccgtgaaggtggtggacgagctggtgaaggtg
atgggccgccacaagccggagaacatcgtgatcgagatggcccgc
gagaaccagaccacgcagaagggccagaagaacagccgcgagcgc
atgaagcgcatcgaggaaggcatcaaggagctgggctcgcagatc
ctgaaggagcaccccgtggagaacacccagctgcagaacgagaag
ctttacctgtactacctgcagaacggccgcgacatgtacgtggac
caggagctggacatcaaccgcctgtccgactacgacgtggaccac
atcgtgccccagagcttcctgaaggacgactcgatcgacaacaag
gtgctgacccgcagcgacaagaaccgcggcaagagcgacaacgtg
ccgtcggaggaagtggtgaagaagatgaagaactactggcgccag
ctgctgaacgccaagctgatcacgcagcgcaagttcgacaacctg
accaaggccgagcgcggtggcctgtcggagctggacaaggcgggc
ttcatcaagcgccagctggtggagacccgccagatcacgaagcac
gtggcgcagatcctggactcccgcatgaacacgaagtacgacgag
aacgacaagctgatccgcgaggtgaaggtgatcaccctgaagtcc
aagctggtcagcgacttccgcaaggacttccagttctacaaggtg
cgcgagatcaacaactaccaccacgcccacgacgcgtacctgaac
gccgtggtgggcaccgcgctgatcaagaagtaccccaagctggag
agcgagttcgtgtacggcgactacaaggtgtacgacgtgcgcaag
atgatcgccaagtcggagcaggagatcggcaaggccaccgcgaag
tacttcttctactccaacatcatgaacttcttcaagaccgagatc
acgctggccaacggcgagatccgcaagcgcccgctgatcgagacc
aacggcgagacgggcgagatcgtgtgggacaagggccgcgacttc
gcgaccgtgcgcaaggtgctgagcatgccccaggtgaacatcgtg
aagaagaccgaggtgcagacgggcggcttctccaaggagagcatc
ctgccgaagcgcaactcggacaagctgatcgcccgcaagaaggac
tgggaccccaagaagtacggcggcttcgactccccgaccgtggcc
tacagcgtgctggtggtggcgaaggtggagaagggcaagtccaag
aagctgaagagcgtgaaggagctgctgggcatcaccatcatggag
cgcagctcgttcgagaagaaccccatcgacttcctggaggccaag
ggctacaaagaggtgaagaaggacctgatcatcaagctgccgaag
tactcgctgttcgagctggagaacggccgcaagcgcatgctggcc
tccgcgggcgagctgcagaagggcaacgagctggccctgcccagc
aagtacgtgaacttcctgtacctggcgtcccactacgagaagctg
aagggctcgccggaggacaacgagcagaagcagctgttcgtggag
cagcacaagcactacctggacgagatcatcgagcagatctcggag
ttctccaagcgcgtgatcctggccgacgcgaacctggacaaggtg
ctgagcgcctacaacaagcaccgcgacaagcccatccgcgagcag
gcggagaacatcatccacctgttcaccctgacgaacctgggcgcc
ccggccgcgttcaagtacttcgacaccacgatcgaccgcaagcgc
tacacctccacgaaagaggtgctggacgcgaccctgatccaccag
agcatcaccggcctgtacgagacgcgcatcgacctgagccagctg
ggcggcgactcccgcgcggacccgaagaagaagcgcaaggtgagc
gctggaggaggtggaagcggaggaggaggaagcggaggaggaggt
agcggatccatgcctcctactaaaagatttcgtattcaagcaaaa
aacatatttcttacatatcctcagtgttctctttcaaaagaagaa
gctcttgagcaaattcaaagaatacaactttcatctaataaaaaa
tatattaaaattgccagagagctacacgaagatgggcaacctcat
ctccacgtcctgcttcaactcgaaggaaaagttcagatcacaaat
atcagattattcgacctggtatccccaaccaggtaattttcatct
ttgtttggccttccaagtgctttttttgctgtttacgggtggaac
ttcagtaaaaatgggatcaaaacatcatatggcataaataaattt
taagaatggcgaactcggggttaccgaatatggcttcctttttca
gtgtttcttagtccattgtacttatgagattgcaggtcagcacat
ttccatccaaacattcagagagctaaatccagctccgacgtcaag
tcctacgtagacaaggacggagacacaattgaatggggagaattc
cagatcgacggtagaagtgctagaggaggtcaacagacagctaac
gactcatatgccaaggcgttaaacgcaacttctcttgaccaagca
cttcaaatattgaaggaagaacaaccaaaggattacttccttcaa
catcacaatcttttgaacaatgctcaaaagatatttcagaggcca
cctgatccatggactccactatttcctctgtcctcattcacaaac
gttcctgaggaaatgcaagaatgggctgatgcatatttcggggtt
gatgccgctgcgcggcctttaagatataatagtatcatagtagag
ggtgattcaagaacagggaagactatgtgggctagatctttaggg
gcccacaattacatcacagggcacttagattttagccctagaacg
tattatgatgaagtggaatacaacgtcattgatgacgtagatccc
acttacttaaagatgaaacactggaaacaccttattggagcacaa
aaggagtggcagacaaacttaaagtatggaaaaccacgtgtcatt
aaaggtggtatcccctgcattatattatgcaatccaggacctgag
agcacataccaacaatttcttgaaaaaccagaaaatgaagccctt
aagtcctggacattacataattcaaccttctgcaaactccaaggt
ccgctctttaataaccaagcagcagcatcctcgcaaggtgactct
accctgtaactgccacttcacaatacaccatgaatgtaatagagg
attttcgcacaggggaacctattactctccatcaggcaacaaatt
ccgtcgaattcgagaatgtaccgaatccactgtatatgaaactcc
tatggttcgagagatacgggccaatctatcaactgaagatacaaa
tcagattcaactacaacctccggagagcgttgaatcttcacaagt
gctggatagagctgacgataactggatcgaacaggatattgactg
gaccccgtttcttgaaggtaagaaatcttttcccatcttgaagtc
acctcaaaccgaacgttaggaaattccaaaatgttttgatagtag
tctacttagtttcaagttttgggtttgtgtatactttcactaata
atatgcgtggaaacattgcaggtcttgaaaaagagactagagata
tacttggataatctaggtttaatttgtattaataatgtaattaga
ggtttaaatcatgtcctgtatgaagaatttacttttgtatcaagt
gtaattcagaaccagagtgttgcaatgaacttgtactaa
exoI gene from bacteriophage λ
(SEQ ID NO: 15)
atgacaccggacattatcctgcagcgtaccgggatcgatgtgaga
gctgtcgaacagggggatgatgcgtggcacaaattacggctcggc
gtcatcaccgcttcagaagttcacaacgtgatagcaaaaccccgc
tccggaaagaagtggcctgacatgaaaatgtcctacttccacacc
ctgcttgctgaggtttgcaccggtgtggctccggaagttaacgct
aaagcactggcctggggaaaacagtacgagaacgacgccagaacc
ctgtttgaattcacttccggcgtgaatgttactgaatccccgatc
atctatcgcgacgaaagtatgcgtaccgcctgctctcccgatggt
ttatgcagtgacggcaacggccttgaactgaaatgcccgtttacc
tcccgggatttcatgaagttccggctcggtggtttcgaggccata
aagtcagcttacatggcccaggtgcagtacagcatgtgggtgacg
cgaaaaaatgcctggtactttgccaactatgacccgcgtatgaag
cgtgaaggcctgcattatgtcgtgattgagcgggatgaaaagtac
atggcgagttttgacgagatcgtgccggagttcatcgaaaaaatg
gacgaggcactggctgaaattggttttgtatttggggagcaatgg
cgatga
sgRNA comprising multiple stem-loops
(SEQ ID NO: 16)
gttttagagctaggccaacatgaggatcacccatgtctgcagggc
ctagcaagttaaaataaggctagtccgttatcaactttaaggagt
ttatatggaaacccttaaagtggcaccgagtcggtgctaccgccg
acaacgcggttttttttttt
triple fusion of E.coli recA gene
(SEQ ID NO: 17)
atggacgagaacaagaagcgcgccctggccgcggccctgggacag
atcgaacgccaattcggcaaaggcgcggtcatgcgcatgggcgac
catgagcgccaggcgatcccggccatctccaccggctccctgggt
ctggacatcgccctcggcatcggcggcctgcccaagggccggatc
gtcgagatctacggtccggaatcctcgggcaagaccaccctgacc
ctctcggtgatcgccgaggcccagaaacagggcgccacctgtgcc
ttcgtcgacgccgagcacgcgctcgatcccgactatgccggcaag
ctgggcgtcaacgtcgacgacctgctggtctcccagccggacacc
ggcgagcaggccctggaaatcaccgacatgctggtgcgctccaac
gcggtcgacgtgatcatcgtcgactccgtggccgcgctggtaccc
aaggccgagatcgaaggcgagatgggcgacgcccacgtcggcctg
caggcacgcctgatgtcccaggcgctgcgcaagatcaccggcaat
atcaagaacgccaactgcctggtcatcttcatcaaccagatccgc
atgaagatcggcgtcatgttcggcaacccggaaaccaccaccggc
ggtaacgcactgaagttctacgcctcggtccgcctggacatccgt
cgtaccggcgcggtgaaggaaggcgacgaggtggtgggtagcgaa
acccgcgtcaaggtggtgaagaacaaggtttccccgccgttccgc
caggccgagttccagatcctctacggtaagggcatctaccgtacc
ggcgagatcatcgatctgggcgtgcaattgggcctggtcgagaag
tccggcgcctggtacagctaccagggcagcaagatcggccagggc
aaggcgaacgccgccaagtacctggaagacaatccggaaatcggt
tcggtactggagaagaccattcgcgaccagttgctggccaagagc
ggcccggtgaaggccgacgccgaagaagtggctgacgccgaagcc
gattcagagctcggagaaggtcaaggacagggacaaggtccagga
cgaggatacgcatataagcttgacgagaacaagaagcgcgccctg
gccgcggccctgggacagatcgaacgccaattcggcaaaggcgcg
gtcatgcgcatgggcgaccatgagcgccaggcgatcccggccatc
tccaccggctccctgggtctggacatcgccctcggcatcggcggc
ctgcccaagggccggatcgtcgagatctacggtccggaatcctcg
ggcaagaccaccctgaccctctcggtgatcgccgaggcccagaaa
cagggcgccacctgtgccttcgtcgacgccgagcacgcgctcgat
cccgactatgccggcaagctgggcgtcaacgtcgacgacctgctg
gtctcccagccggacaccggcgagcaggccctggaaatcaccgac
atgctggtgcgctccaacgcggtcgacgtgatcatcgtcgactcc
gtggccgcgctggtacccaaggccgagatcgaaggcgagatgggc
gacgcccacgtcggcctgcaggcacgcctgatgtcccaggcgctg
cgcaagatcaccggcaatatcaagaacgccaactgcctggtcatc
ttcatcaaccagatccgcatgaagatcggcgtcatgttcggcaac
ccggaaaccaccaccggcggtaacgcactgaagttctacgcctcg
gtccgcctggacatccgtcgtaccggcgcggtgaaggaaggcgac
gaggtggtgggtagcgaaacccgcgtcaaggtggtgaagaacaag
gtttccccgccgttccgccaggccgagttccagatcctctacggt
aagggcatctaccgtaccggcgagatcatcgatctgggcgtgcaa
ttgggcctggtcgagaagtccggcgcctggtacagctaccagggc
agcaagatcggccagggcaaggcgaacgccgccaagtacctggaa
gacaatccggaaatcggttcggtactggagaagaccattcgcgac
cagttgctggccaagagcggcccggtgaaggccgacgccgaagaa
gtggctgacgccgaagccgattcaggatccggagaaggtcaagga
cagggacaaggtccaggacgaggatacgcatatgcatgcgacgag
aacaagaagcgcgccctggccgcggccctgggacagatcgaacgc
caattcggcaaaggcgcggtcatgcgcatgggcgaccatgagcgc
caggcgatcccggccatctccaccggctccctgggtctggacatc
gccctcggcatcggcggcctgcccaagggccggatcgtcgagatc
tacggtccggaatcctcgggcaagaccaccctgaccctctcggtg
atcgccgaggcccagaaacagggcgccacctgtgccttcgtcgac
gccgagcacgcgctcgatcccgactatgccggcaagctgggcgtc
aacgtcgacgacctgctggtctcccagccggacaccggcgagcag
gccctggaaatcaccgacatgctggtgcgctccaacgcggtcgac
gtgatcatcgtcgactccgtggccgcgctggtacccaaggccgag
atcgaaggcgagatgggcgacgcccacgtcggcctgcaggcacgc
ctgatgtcccaggcgctgcgcaagatcaccggcaatatcaagaac
gccaactgcctggtcatcttcatcaaccagatccgcatgaagatc
ggcgtcatgttcggcaacccggaaaccaccaccggcggtaacgca
ctgaagttctacgcctcggtccgcctggacatccgtcgtaccggc
gcggtgaaggaaggcgacgaggtggtgggtagcgaaacccgcgtc
aaggtggtgaagaacaaggtttccccgccgttccgccaggccgag
ttccagatcctctacggtaagggcatctaccgtaccggcgagatc
atcgatctgggcgtgcaattgggcctggtcgagaagtccggcgcc
tggtacagctaccagggcagcaagatcggccagggcaaggcgaac
gccgccaagtacctggaagacaatccggaaatcggttcggtactg
gagaagaccattcgcgaccagttgctggccaagagcggcccggtg
aaggccgacgccgaagaagtggctgacgccgaagccgattaa
triple fusion of Arabidopsis rad51 gene
(SEQ ID NO: 18)
atgacgacgatggagcagcgtagaaaccagaatgctgtccaacaa
caagacgatgaagaaacccagcacggacctttccctgtcgaacag
cttcaggcagcaggtattgcttctgttgatgtaaagaagcttagg
gatgctggtctctgtactgttgaaggtgttgcttatactccgagg
aaggatctcttgcagattaaaggaattagtgatgccaaggttgac
aagattgtagaagcagcttcaaagctagttcctctggggttcaca
agtgcgagccagctccatgctcagagacaggaaattattcagatt
acctctggatcacgggagctcgataaagttctagaaggaggtatt
gaaactggttccatcacagagttatatggtgagttccgctctgga
aagactcagctgtgccatacactgtgtgtgacttgtcaacttccc
atggatcaaggaggtggagagggaaaggccatgtacattgatgct
gagggaacattcaggccacaaagactcttacagatagctgacagg
tttggattaaatggagctgatgtactagaaaacgttgcctatgcg
agggcgtataatacagatcatcagtcaaggcttttgcttgaagca
gcatcaatgatgattgaaacaaggtttgctctcctgattgtcgat
agtgctaccgctctctacagaacagatttctctggaaggggagag
ctttcggctcgacaaatgcatcttgcaaagttcttgagaagtctt
cagaagttagcagatgagtttggtgtggctgttgttataacaaac
caagtagttgcgcaagtagatggttcagctctttttgctggtccc
caatttaagccgattggtgggaatatcatggctcatgccaccaca
acaaggttggcgttgaggaaaggaagagcagaggagagaatctgt
aaagtgataagctcgccatgtttgccagaagcggaagctcgattt
caaatatctacagaaggtgtaacagattgcaaggatgagctcgga
gaaggtcaaggacagggacaaggtccaggacgaggatacgcatat
aagcttatgacgacgatggagcagcgtagaaaccagaatgctgtc
caacaacaagacgatgaagaaacccagcacggacctttccctgtc
gaacagcttcaggcagcaggtattgcttctgttgatgtaaagaag
cttagggatgctggtctctgtactgttgaaggtgttgcttatact
ccgaggaaggatctcttgcagattaaaggaattagtgatgccaag
gttgacaagattgtagaagcagcttcaaagctagttcctctgggg
ttcacaagtgcgagccagctccatgctcagagacaggaaattatt
cagattacctctggatcacgggagctcgataaagttctagaagga
ggtattgaaactggttccatcacagagttatatggtgagttccgc
tctggaaagactcagctgtgccatacactgtgtgtgacttgtcaa
cttcccatggatcaaggaggtggagagggaaaggccatgtacatt
gatgctgagggaacattcaggccacaaagactcttacagatagct
gacaggtttggattaaatggagctgatgtactagaaaacgttgcc
tatgcgagggcgtataatacagatcatcagtcaaggcttttgctt
gaagcagcatcaatgatgattgaaacaaggtttgctctcctgatt
gtcgatagtgctaccgctctctacagaacagatttctctggaagg
ggagagctttcggctcgacaaatgcatcttgcaaagttcttgaga
agtcttcagaagttagcagatgagtttggtgtggctgttgttata
acaaaccaagtagttgcgcaagtagatggttcagctctttttgct
ggtccccaatttaagccgattggtgggaatatcatggctcatgcc
accacaacaaggttggcgttgaggaaaggaagagcagaggagaga
atctgtaaagtgataagctcgccatgtttgccagaagcggaagct
cgatttcaaatatctacagaaggtgtaacagattgcaaggatgga
tccggagaaggtcaaggacagggacaaggtccaggacgaggatac
gcatatgcatgcatgacgacgatggagcagcgtagaaaccagaat
gctgtccaacaacaagacgatgaagaaacccagcacggacctttc
cctgtcgaacagcttcaggcagcaggtattgcttctgttgatgta
aagaagcttagggatgctggtctctgtactgttgaaggtgttgct
tatactccgaggaaggatctcttgcagattaaaggaattagtgat
gccaaggttgacaagattgtagaagcagcttcaaagctagttcct
ctggggttcacaagtgcgagccagctccatgctcagagacaggaa
attattcagattacctctggatcacgggagctcgataaagttcta
gaaggaggtattgaaactggttccatcacagagttatatggtgag
ttccgctctggaaagactcagctgtgccatacactgtgtgtgact
tgtcaacttcccatggatcaaggaggtggagagggaaaggccatg
tacattgatgctgagggaacattcaggccacaaagactcttacag
atagctgacaggtttggattaaatggagctgatgtactagaaaac
gttgcctatgcgagggcgtataatacagatcatcagtcaaggctt
ttgcttgaagcagcatcaatgatgattgaaacaaggtttgctctc
ctgattgtcgatagtgctaccgctctctacagaacagatttctct
ggaaggggagagctttcggctcgacaaatgcatcttgcaaagttc
ttgagaagtcttcagaagttagcagatgagtttggtgtggctgtt
gttataacaaaccaagtagttgcgcaagtagatggttcagctctt
tttgctggtccccaatttaagccgattggtgggaatatcatggct
catgccaccacaacaaggttggcgttgaggaaaggaagagcagag
gagagaatctgtaaagtgataagctcgccatgtttgccagaagcg
gaagctcgatttcaaatatctacagaaggtgtaacagattgcaag
gattaactagtg
MS2-derived stem-loop for binding
(SEQ ID NO: 19)
ggccaacatgaggatcacccatgtctgcagggcc
PP7-derived stem-loop for binding
(SEQ ID NO: 20)
taaggagtttatatggaaaccctta
P22-derived stem-loop for binding B-box
(SEQ ID NO: 21)
accgccgacaacgcggt
MS2 bacteriophage coat protein
(SEQ ID NO: 22)
atggcttcaaactttactcagttcgtgctcgtggacaatggtggg
acaggggatgtgacagtggctccttctaatttcgctaatgggggg
cagagtggatcagctccaactcacggagccaggcctacaaggtga
catgcagcgtcaggcagtctagtgcccagaagagaaagtatacca
tcaaggtggaggtccccaaagtggctacccagacagtgggcggag
tcgaactgcctgtcgccgcttggagatcctacctgaacatggaac
tcactatcccaattttcgctaccaattctgactgtgaactcatcg
tgaaggcaatgcaggggctcctcaaagacggcaatcctatccctt
ccgccatcgccgctaactcaggca
PP7 bacteriophage coat protein
(SEQ ID NO: 23)
atgtccaaaaccatcgttctttcggtcggcgaggctactcgcact
ctgactgagatccagtccaccgcagaccgtcagatcttcgaagag
aaggtcgggcctctggtgggtcggctgcgcctcacggcttcgctc
cgtcaaaacggagccaagaccgcgtatcgagtcaacctaaaactg
gatcaggcggacgtcgttgattgctccaccagcgtctgcggcgag
cttccgaaagtgcgctacactcaggtatggtcgcacgacgtgaca
atcgttgcgaatagcaccgaggcctcgcgcaaatcgttgtacgat
ttgaccaagtccctcgtcgcgacctcgcaggtcgaagatcttgtc
gtcaaccttgtgccgctgggccgttaa
P22 bacteriophage coat protein
(SEQ ID NO: 24)
atgacggttatcacctacgggaagtcaacgtttgcaggcaatgct
aaaactcgccgtcatgagcggcgcagaaagctagccatagagcgc
gacaccatctgcaatatcatcgattcaatttttggctgcgatgct
cctgatgcttctcaggaagttaaagccaaaagaattgaccgtgtc
accaaagccatttcgcttgccggaacgcgtcagaaggaagttgaa
ggaggatctgtacttcttccaggcgtagcactttacgcggctggt
catcgtaagagcaaacaaataacagcgaggtaa
MSH2 dominant-negative allele gene sequence
(SEQ ID NO: 25)
atggctgggttaaggcaggatcttagacagcatctgaagcgaatc
tcagatgttgagaggcttttgcgcagtctcgagagaagaagaggt
gggttacagcacattattaaactctatcaggtactttccgcactt
caatctgcttctctcaatgttaacaaaattgcattttcattgtcc
taaatgtgtttatgcaactctgaagttataggtatgttattaagt
tcattactaattaagtcttcatcttttctctgcagtcagctataa
ggcttcccttcatcaaaacagctatgcaacagtacaccggagaat
tcgcatcactcatcagcgagaggtacctgaaaaagcttgaggctt
tatcagatcaagatcaccttggaaagttcatcgatttggttgagt
gctctgtagatcttgaccagctagaaaatggagaatacatgatat
cttcaaactacgacaccaaattggcatctctgaaagatcagaaag
aattgctggagcagcagattcacgaattgcacaaaaagacagcga
tagaacttgatcttcaggtcgacaaggctcttaaacttgacaaag
cagcgcaatttgggcatgtcttcaggatcacgaagaaggaagagc
caaagatcaggaagaagctgacgacacagtttatagtgctggaga
ctcgcaaagacggagtgaagttcacaaacacaaagctaaaaaaac
tgggcgaccagtaccaaagtgttgtggatgattataggagctgtc
aaaaggagctcgttgatcgtgtagttgagactgttaccagcttct
ctgaggtatgtttagttattcatattaagcattggactgttacag
aattggttgtttaaaatcatagtaaactatatgtggaatttatat
gtatattgtatggttataggtatttgaggacttagctgggttact
ttctgaaatggatgttttgttaagctttgctgatttggctgccag
ttgccctactccatactgtaggccagaaatcacctctttggttag
tacaatctcaagttgattattttgttctgaaaatgaatagttttt
tctttccaagtttatgacataatgttgagagcacggttaataaat
tgtaggatgctggagatattgtactagaaggaagcagacatccat
gtgtagaagctcaagattgggtgaatttcataccaaatgattgca
gactcgtaagtattgaatgtggtaaataaactgagacgtctttgt
ttttcttgtttcccttttgacttgaacaaatacttgtttgccctt
tactgttctttgaaatcagatgagagggaagagttggtttcaaat
agtaacagggcctaacatgggagataagtccactttcatccgcca
ggtatgatgatttcctctagttcagttttgcttcatagacgtatg
actaaagtcggtttccggccattataaatcccaggttggtgtgat
tgtgctgatggctcaagttggttcctttgttccttgtgataaagc
atcaatttccataagagactgcatctttgcccgtgtaggagcagg
cgattgccaagtgagtttaagtttagccctcaatgaacgaaaaac
tgctgatatcctgaacacccttattccaactttttttcctttggt
gtgttagctgcgtggagtgtcaacttttatgcaagaaatgcttga
aaccgcatcgatattgaaaggcgctactgataagtcactgataat
tatcgatgaacttggtcgtggaacatcaacttatgatggttttgg
ttagtttctctgcaatttctcttctttcatttggatgtttttagt
aagttttctattatatattcatttttatggtcatatgtgagattt
cagtgctcttgacatcatcgtggtgaatatatcaggtttagcttg
ggctatatgtgagcatctggttcaagtgaaaagagcaccaactct
gtttgctactcacttccatgaacttactgccttggctcaagcaaa
ctctgaggtctctggtaacactgttggtgtggcaaacttccatgt
cagcgctcacattgacactgaaagccgcaaactcaccatgcttta
caaggtctggtttataaattaaaaaattgctgatctgttgcagtt
aaaagtgtctctgtttttatgtttaatctaaattacttatttgat
tttcttacaaagatgaaattgaaattaattttgtgtggtgtgttg
tttgtctggttaggttgaaccaggggcctgtgaccagagctttgg
gattcatgtggcggaatttgccaacttccctgaaagcgtcgtggc
cctcgcaagagagaaagctgcagagctggaagatttctctccctc
ctcgatgataatcaacaatgaggtcttgattcatttccccctttg
tttttggttgatgatggaatcattctatcattcacccattctgca
gtttatgctatattattataaatctatgtgacaaagatttaattc
tcgtattgttgtttgcaggagagtgggaagagaaagagcagagaa
gatgatccagatgaagtatcaagaggggcagagcgagctcacaag
tttctgaaagagtttgcagcgatgccacttgataaaatggagctt
aaagattcacttcaacgggtacgtgagatgaaagatgagctagag
aaagatgctgcagactgccactggctcaggcagtttctgtgaaga
acccctga
Bacteriophage T4 endonuclease VII, T4E7
(SEQ ID NO: 26)
Atgttattgactggcaaattatacaaagaagaaaaacagaaattt
tatgatgcacaaaacggtaaatgcttaatttgccaacgagaacta
aatcctgatgttcaagctaatcacctcgaccatgaccatgaatta
aatggaccaaaagcaggaaaggtgcgtggattgctttgtaatcta
tgcaatgctgcagaaggtcaaatgaagcataaatttaatcgttct
ggcttaaagggacaaggtgttgattatcttgaatggttagaaaat
ttacttacttatttaaaatccgattacacccaaaataatattcac
cctaactttgttggagataaatcaaaggaattttctcgtttagga
aaagaggaaatgatggccgagatgcttcaaagaggatttgaatat
aatgaatctgacaccaaaacacaattaatagcttcattcaagaag
cagcttagaaagagtttaaaatga
Yeast CCE1 resolvase,
(SEQ ID NO: 27)
atgtcgacagcacagaaagctaagatattgcaactcatcgattcc
tgctgccaaaatgcaaaaagcacacaactgaaatctttatcattt
gttattggagcagtaaatggcacgacgaaagaagctaaaagaacc
tacattcaagaacagtgtgaatttttggagaagttacgacaacaa
aagataagagagggaagaattaacatattgtctatggatgctggt
gtttctaactttgctttctctaagatgcaattgctcaataatgat
ccgctccctaaagtactagactggcaaaagataaatctagaggag
aaattttttcaaaacctcaaaaagttaagcttgaatcctgctgaa
acttctgagcttgtatttaaccttacggagtatttatttgaatct
atgccgataccagatatgtttacaattgaaaggcaacgtaccaga
actatgtcttcgaggcatattttagacccaattttaaaagtgaat
attctcgaacagattcttttctctaacttggaaaataaaatgaag
tatacgaataaaataccgaatacgtccaagttgaggtatatggta
tgttcgtccgatccacatcggatgacttcatattggtgcattcca
agagaagagacaccgaccagttcaaaaaagttaaaatctaacaaa
catagcaaagattctcgaataaagctagtgaaaaaaatactttca
acctcaatactagaaggtaattcaactagttctacaaaactggtc
gagttcataggagtttggaataataggataagaaatgcccttacc
aaaaaaaaaagtttcaagctatgtgatatactagagatccaagat
aattcgggggtgagaaaagatgacgatttggcagattcattcctc
cattgtttgtcttggatggagtggttaaaaaattatgaaagtatt
actgaactcttgaattcaaaaacactggttaaaacacagttcgga
caggtgtttgaattttgtgaaaataaggtacaaaagctgaaattt
ttgcagaacacttacaacaatgactaa
Arabidopsis AtGEN1 resolvase,
(SEQ ID NO: 28)
atgggtgtgggaggcaatttctgggatttgctgagaccatatgct
cagcaacaaggctttgattttctcagaaacaaacgagtcgctgtt
gatctctccttctggatcgttcagcatgaaaccgctgttaagggt
ttcgtccttaaacctcacctccgactcactttcttccgtactatc
aacctcttctcaaagtttggagcgtacccggtttttgtggttgat
ggaacaccatcacctttgaaatctcaggcgagaatctccaggttt
ttccgttcttctggaattgatacttgtaatctacctgtgattaaa
gatggtgtctcggttgagagaaacaagctgttttctgaatgggtt
agggaatgtgtggagctactcgaattgctcggtattccggtgctg
aaagctaatggtgaggctgaagctctctgtgcacagttaaacagc
caaggttttgtggatgcttgcattactcctgatagtgatgctttc
ctttttggtgctatgtgcgtgatcaaagacatcaagcctaattca
agagaaccttttgaatgctaccatatgtcacatatcgagtctggc
ctcggtttgaagcggaaacacttgattgctatttctctattggtg
ggaaacgattatgattcaggcggtgttcttgggattggtgtggat
aaagcactgcgcattgttcgtgagttttctgaagaccaagtactt
gaaagactacaggacattggaaatgggttgcaacctgcagttcct
ggtggaatcaaatccggggatgatggtgaagaattccgctcagag
atgaaaaaaagatctcctcactgttcccgttgtggacacctgggc
agcaagagaactcattttaagtcctcttgtgagcactgcggttgt
gatagtggttgcattaaaaaaccattagggtttagatgtgaatgc
tccttttgttccaaggatcgagatttaagggaacaaaagaaaacc
aatgattggtggatcaaagtctgcgataagattgctctagcgcca
gagtttcccaacagaaagattattgaactttatctatccgatggt
ttgatgacaggagatggatcgtcaatgtcttggggaactcctgat
actggaatgctagtggatctcatggttttcaaactgcactgggac
ccatcttatgttagaaaaatgttgcttccgatgctgtcgaccatt
tatctgagagaaaaggcaagaaacaacacaggatacgctttgttg
tgtgatcaatacgaatttcattcaatcaagtgcataaaaactaga
tatgggcatcagtcctttgtaataaggtggagaaaacccaaatct
acaagtggttatagtcatagtcacagcgagccagaagaatcaatt
gttgtattggaagaagaagaagagtctgttgatccgttggatggt
ttaaatgaacctcaggtgcaaaatgataatggtgactgcttcttg
ctaactgatgaatgcataggacttgttcagtctgctttccctgat
gaaacagagcattttctacatgagaagaaactgagagagtcgaaa
aagaagaatgtttctgaagaagaaacagcaacaccaagagcaaca
acaatgggtgtacaaagaagcattaccgatttctaccgttcagcg
aagaaagcagcagcaggtcaaagtatagagacaggcgggagttca
aaagcttctgcggaaaagaagagacaggcaacttctactagtagt
agtaaccttacaaagtcggtcaggcgtcgtctcttgtttggatag
Selection donor vector for introduction of
premature stop codon into the tobacco PDS
gene
(SEQ ID NO: 29)
cttcaggaagtttcccgctcaaaaacgtcacatcattcaacagtc
cccttccacgtgtcacgttttgattgggtgcccatttttttcggg
ccatccggtaatattataacggatggcccttatgaaagttgtacta
atatatataatactccaatactccaatagatatgttaatatgctaa
gccacgtcatcggagtacaaacatggaattcctggctgatgctggt
cacaaaccgatattgctggaggcaagagatgtcctaggtggaaagg
tgaagaatatccatgctttcctttaattttattcctttttctttt
gtgtccttccctattgatagtcccttttcaggaaggcttctattt
gttttgtttaaaatcatttttcatactctttaaacattcagttgc
tcaaacaattgcaagggtgttcactattcctatttttgactgtct
tcctttctctcagtttagttttattcctctctctctctctctctc
tatttttggaggaaatagatctgtcctaaaaatttccagctttac
tactaatagtgttaattgtcgagaaaatagtacagcatattaggt
aaaagatatggaaagtatattattattattattattattattatt
attattattattattattattattattattattattattattatt
ctctattattttaagattgagtcaattttacctgtcctgttggtt
gcatttctcatataaacattcttttctgtgagatgctatgtgaat
tagctgatgtttttggtatagagcactatgttagtcagttttatc
ttactgaagcagtcaccaagaatctagttgtataggctaaaagat
tgaattagcattaatctttatgtgttctgcacctgaatacttata
cctaccttttaggtagctgcatagaaagatgatgatagagattgg
tatgagactgggttgcacatattctgtaagtttgactcctcaaga
atgcatactttaatcttctagtacaacagtttctttcaagatctc
ttttgtccattaatcagatagctatccctgtttgtcttttgcaaa
tagccaatatgtcagtcgatctgtattctgccttgcctatctttt
tttatctgttaatttcgtatggtgactcatacaagttggtgcatc
tcctttaagttggggcttacccaaatatgcagaacttgtttggag
aactagggataaacgatcggttgcagtggaaggaacattcaatga
tatttgcgatgcctaacaagccagggggatcccgcagggaggcaa
acaatgatatcacaactctcctgacgcgtcatcgtcggctacagc
ctcgggaattgctacctagctcgagcaagatccaaggagatataa
caatggcttcctcctggattgaacaagatggattgcacgcaggtt
ctccggccgcttgggtggagaggctattcggctatgactgggcac
aacagacaatcggctgctctgatgccgccgtgttccggctgtcag
cgcaggggcgcccggttctttttgtcaagaccgacctgtccggtg
ccctgaatgaactccaagacgaggcagcgcggctatcgtggctgg
ccacgacgggcgttccttgcgcagctgtgctcgacgttgtcactg
aagcgggaagggactggctgctattgggcgaagtgccggggcagg
atctcctgtcatctcaccttgctcctgccgagaaagtatccatca
tggctgatgcaatgcggcggctgcatacgcttgatccggctacct
gcccattcgaccaccaagcgaaacatcgcatcgagcgagcacgta
ctcggatggaagccggtcttgtcgatcaggatgatctggacgaag
agcatcaggggctcgcgccagccgaactgttcgccaggctcaagg
cgcggatgcccgacggcgaggatctcgtcgtgacccacggcgatg
cctgcttgccgaatatcatggtggaaaatggccgcttttctggat
tcatcgactgtggccggctgggtgtggcggaccgctatcaggaca
tagcgttggctacccgtgatattgctgaagagcttggcggcgaat
gggctgaccgcttcctcgtgctttacggtatcgccgctcccgatt
cgcagcgcatcgccttctatcgccttcttgacgagttcttctgaa
ctagtgatcgttcaaacatttggcaataaagtttcttaagattga
atcctgttgccggtcttgcgatgattatcatataatttctgttga
attacgttaagcatgtaataattaacatgtaatgcatgacgttat
ttatgagatgggttttttgattagagtcccgcaattatacattta
atacgcgatagaaaacaaaatatagcgcgcaaactaggataaatt
atcgcgcgcggtgtcatctatgttactagatcgacccgggcttca
ggaagtttcccgctcaaaaacgtcacatcattcaacagtcccctt
ccacgtgtcacgttttgattgggtgcccatttttttcgggccatc
cggtaatattataacggatggcccttatgaaagttgtactaatat
atataatactccaatactccaatagatatgttaatatgctaagcc
acgtcatcggagtaca
BOR1-ALS LFS-nptII-ALS RFS BOR2
(SEQ ID NO: 30)
tcctgtactccgatgacgtggcttagcatattaacatatctattg
gagtattggagtattatatatattagtacaactttcataagggcc
atccgttataatattaccggatggcccgaaaaaaatgggcaccca
atcaaaacgtgacacgtggaaggggactgttgaatgatgtgacgt
ttttgagcgggaaacttcctgaaggcgcgccggagagtaaggaag
gtaaactgaagttggatttttctgcttggaggcaggagttgacgg
tgcagaaagtgaagtacccgttgaattttaaaacttttggtgatg
ctattcctccgcaatatgctatccaggttctagatgagttaacta
atgggagtgctattataagtaccggtgttgggcagcaccagatgt
gggctgctcaatattataagtacagaaagccacgccaatggttga
catctggtggattaggagcgatgggatttggtttgcccgctgcta
ttggtgcggctgttggaagacctgatgaagttgtggttgacattg
atggtgatggcagtttcatcatgaatgtgcaggagctagcaacta
ttaaggtggagaatctcccagttaagattatgttactgaataatc
aacacttgggaatggtggttcaattggaggatcggttctataagg
ctaacagagcacacacatacctggggaatccttctaatgaggcgg
agatctttcctaatatgttgaaatttgcagaggcttgtggcgtac
ctgctgcgagagtgacacacagggatgatcttagagcggctattc
aaaagatgttagacactcctgggccatacttgttggatgtgattg
tacctcatcaggaacatgttctacctatgattcccagtggcgggg
ctttcaaagatgtgatcacagagggtgacagaagttcctatggat
cccgcagggaggcaaacaatgatatcacaactctcctgacgcgtc
atcgtcggctacagcctcgggaattgctacctagctcgagcaaga
tccaaggagatataacaatggcttcctcctggattgaacaagatg
gattgcacgcaggttctccggccgcttgggtggagaggctattcg
gctatgactgggcacaacagacaatcggctgctctgatgccgccg
tgttccggctgtcagcgcaggggcgcccggttctttttgtcaaga
ccgacctgtccggtgccctgaatgaactccaagacgaggcagcgc
ggctatcgtggctggccacgacgggcgttccttgcgcagctgtgc
tcgacgttgtcactgaagcgggaagggactggctgctattgggcg
aagtgccggggcaggatctcctgtcatctcaccttgctcctgccg
agaaagtatccatcatggctgatgcaatgcggcggctgcatacgc
ttgatccggctacctgcccattcgaccaccaagcgaaacatcgca
tcgagcgagcacgtactcggatggaagccggtcttgtcgatcagg
atgatctggacgaagagcatcaggggctcgcgccagccgaactgt
tcgccaggctcaaggcgcggatgcccgacggcgaggatctcgtcg
tgacccacggcgatgcctgcttgccgaatatcatggtggaaaatg
gccgcttttctggattcatcgactgtggccggctgggtgtggcgg
accgctatcaggacatagcgttggctacccgtgatattgctgaag
agcttggcggcgaatgggctgaccgcttcctcgtgctttacggta
tcgccgctcccgattcgcagcgcatcgccttctatcgccttcttg
acgagttcttctgaactagtgtttgagaagctacagagctagttc
taggccttgtattatctaaaataaacttctattaaaccaaaaatg
ttatgtctattagtttgttattagtttttccgtggctttgctcat
tgtcagtgttgtactattaagtagttgatatttatgtttgcttta
agttttgcatcatctcgctttggttttgaatgtgaaggatttcag
caatgtttcattctctattcgcaacatccagtcggtatccggagc
tctatgtagtatgtctggagattaatttctagtggagtagtttag
tgcgataaagttagcttgttccacatttttatttcgtaacctggg
tcagattggaacttcctctttaggttggatgcaatccctatttgg
gctttctcttaatttcattattgaaattgttggcttttaatctga
gcaagttgatttgcagctttctctcttgagtcctagcgagcaata
cgttatctctgtctcctatttcttagtggataatcttatgatgga
aatctgtggagataggaaagcggccgctcctgtactccgatgacg
tggcttagcatattaacatatctattggagtattggagtattata
tatattagtacaactttcataagggccatccgttataatattacc
ggatggcccgaaaaaaatgggcacccaatcaaaacgtgacacgtg
gaaggggactgttgaatgatgtgacgtttttgagcgggaaacttc
ctgaagccg
BOR1-PPOX1 LFS-nptll-PPOX1 RFS BOR2
(SEQ ID NO: 31)
tcctgtactccgatgacgtggcttagcatattaacatatctattg
gagtattggagtattatatatattagtacaactttcataagggcc
atccgttataatattaccggatggcccgaaaaaaatgggcaccca
atcaaaacgtgacacgtggaaggggactgttgaatgatgtgacgt
ttttgagcgggaaacttcctgaagggcgcgccacaataacatcag
gatttgattcatcttaaatataatacttgcgtttcatatgcagga
acgatatatagttcatcactcttccctaaccgtgccccaaaaggt
cgggtgctactcttgaacatgattggaggagcaaaaaatccggaa
attttgtctaaggtaaggcatttaaattgagcgcaacttcaagtc
ctgccggtattgatttatcataatgaatattcatgggctggccag
ttggggcatcgcttgaaggttcctatggtaacaaacagaaaactg
agaatgaccttatcccctgatctcttttggttgcaaatacgttaa
ccttgcagtgatgaggagaatgttttcagtgttattttctctaga
ttaatggcgtagttggtttggattacaaaaccgtttaggcttata
tgtgattagagtctccaggtttttgatctagcagtaagagcaaaa
tgtgtgatgttttaaaaaaacagtcaagtgattacaaatactcag
aatagtttcaccataaagaactaaagagctaagaagattgtggtt
tctctttcctctatgttcgactgcattttgtgggttttccatagc
atgactttcttttatggtacttgtacgaaagcatttcgctgttat
tctttatgcttcagggcctagaataaattgctttgagatttgatc
cctcattcttcttgcagacggagagccaacttgtggaagtagttg
atcgtgacctcagaaaaatgcttatagaacccaaagcacaagatc
cccttgttgtgggtgtgcgagtatggccacaagctatcccacagt
ttttggttggtcatctgggtacgctaagtactgcaaaagctgcta
tgagtgataatgggcttgaagggctgtttcttgggggtaattatg
tgtcaggtgtagcattggggaggtgtgttgaaggagcttatgaag
ttgcatctgaggtaacaggatttctgtctcggtatgcttacaaag
gatcccgcagggaggcaaacaatgatatcacaactctcctgacgc
gtcatcgtcggctacagcctcgggaattgctacctagctcgagca
agatccaaggagatataacaatggcttcctcctggattgaacaag
atggattgcacgcaggttctccggccgcttgggtggagaggctat
tcggctatgactgggcacaacagacaatcggctgctctgatgccg
ccgtgttccggctgtcagcgcaggggcgcccggttctttttgtca
agaccgacctgtccggtgccctgaatgaactccaagacgaggcag
cgcggctatcgtggctggccacgacgggcgttccttgcgcagctg
tgctcgacgttgtcactgaagcgggaagggactggctgctattgg
gcgaagtgccggggcaggatctcctgtcatctcaccttgctcctg
ccgagaaagtatccatcatggctgatgcaatgcggcggctgcata
cgcttgatccggctacctgcccattcgaccaccaagcgaaacatc
gcatcgagcgagcacgtactcggatggaagccggtcttgtcgatc
aggatgatctggacgaagagcatcaggggctcgcgccagccgaac
tgttcgccaggctcaaggcgcggatgcccgacggcgaggatctcg
tcgtgacccacggcgatgcctgcttgccgaatatcatggtggaaa
atggccgcttttctggattcatcgactgtggccggctgggtgtgg
cggaccgctatcaggacatagcgttggctacccgtgatattgctg
aagagcttggcggcgaatgggctgaccgcttcctcgtgctttacg
gtatcgccgctcccgattcgcagcgcatcgccttctatcgccttc
ttgacgagttcttctgaactagtaacctgtctgggggtactgcta
ggtccaaaccttgttagtaatacgatcatgccttgggaatattgg
catgtgcctaaaagttttgctcgttagagttattttagccttggt
aaatgatttgtacttgatatcagtcgttttctttgagataaaatg
ttcctgttcaggaaaatataatgtatatcaattttaaacacttga
atgttgaagatcattttttcccctcagcttacccataaatgtgaa
aggtcctttgcttctgcatggtgagactgccgatatattttctcc
aacttcctatggttaaatatggtttgccttgtcattctttgtttt
ctttgggagattatttattccacgaccagaagtaagggagtatac
cacattgattcaagggctgatacttgtggcaacaaagactaactg
tgcaagggtaacagtgagagtatatttattactcacttctaataa
agagcaaagttaagggaattcgatgatttaggagcaaattaggcc
ttttcccttgagttaataaagagatgctattaaaattatacttct
ttgtatttaaatttgatataggtaatatgttctgtcatacaacat
ttattaggtgtaattcaatagtacttttaaaaaataaagtttttt
aacatgaaatcaaaaatagatcaggactgctcttgtatagatcta
gtctagttaaaataaataactgcataagtgtttgcccacactaat
catataggcacccaaaactctcctcctttttcgtccccaaacgtc
tcctccctcgctgcggccgctcctgtactccgatgacgtggctta
gcatattaacatatctattggagtattggagtattatatatatta
gtacaactttcataagggccatccgttataatattaccggatggc
ccgaaaaaaatgggcacccaatcaaaacgtgacacgtggaagggg
actgttgaatgatgtgacgtttttgagcgggaaacttcctgaagc
cg
nptII gene
(SEQ ID NO: 32)
atggaacaagatggattgcacgcaggttctccggccgcttgggtg
gagaggctattcggctatgactgggcacaacagacaatcggctgc
tctgatgccgccgtgttccggctgtcagcgcaggggcgcccggtt
ctttttgtcaagaccgacctgtccggtgccctgaatgaactccaa
gacgaggcagcgcggctatcgtggctggccacgacgggcgttcct
tgcgcagctgtgctcgacgttgtcactgaagcgggaagggactgg
ctgctattgggcgaagtgccggggcaggatctcctgtcatctcac
cttgctcctgccgagaaagtatccatcatggctgatgcaatgcgg
cggctgcatacgcttgatccggctacctgcccattcgaccaccaa
gcgaaacatcgcatcgagcgagcacgtactcggatggaagccggt
cttgtcgatcaggatgatctggacgaagagcatcaggggctcgcg
ccagccgaactgttcgccaggctcaaggcgcggatgcccgacggc
gaggatctcgtcgtgacccacggcgatgcctgcttgccgaatatc
atggtggaaaatggccgcttttctggattcatcgactgtggccgg
ctgggtgtggcggaccgctatcaggacatagcgttggctacccgt
gatattgctgaagagcttggcggcgaatgggctgaccgcttcctc
gtgctttacggtatcgccgctcccgattcgcagcgcatcgccttc
tatcgccttcttgacgagttcttctg
tobacco ALS sgRNA
(SEQ ID NO: 33)
gatgtgatcacagagggtgacgttttagagctaggccaacatgag
gatcacccatgtctgcagggcctagcaagttaaaataaggctagt
ccgttatcaacttggccaacatgaggatcacccatgtctgcaggg
ccaagtggcaccgagtcggtgctttttttttttgcggccatcttg
ctgaaaaa
tobacco PPOX1 sgRNA
(SEQ ID NO: 34)
ggctgcatggaaagatgatgagttttagagctaggccaacatgag
gatcacccatgtctgcagggcctagcaagttaaaataaggctagt
ccgttatcaacttggccaacatgaggatcacccatgtctgcaggg
ccaagtggcaccgagtcggtgcttttttttttt
Tobacco PDS donor for knock out mutagenesis
(SEQ ID NO: 35)
Ctggctgatgctggtcacaaaccgatattgctggaggcaagagat
gtcctaggtggaaaggtgaagaatatccatgctttcctttaattt
tattcctttttcttttgtgtccttccctattgatagtcccttttc
aggaaggcttctatttgttttgtttaaaatcatttttcatactct
ttaaacattcagttgctcaaacaattgcaagggtgttcactattc
ctatttttgactgtcttcctttctctcagtttagttttattcctc
tctctctctctctctctatttttggaggaaatagatctgtcctaa
aaatttccagctttactactaatagtgttaattgtcgagaaaata
gtacagcatattaggtaaaagatatggaaagtatattattattat
tattattattattattattattattattattattattattattat
tattattattattattctctattattttaagattgagtcaatttt
acctgtcctgttggttgcatttctcatataaacattcttttctgt
gagatgctatgtgaattagctgatgtttttggtatagagcactat
gttagtcagttttatcttactgaagcagtcaccaagaatctagtt
gtataggctaaaagattgaattagcattaatctttatgtgttctg
cacctgaatacttatacctaccttttaggtagctgcatagaaaga
tgatgatagagattggtatgagactgggttgcacatattctgtaa
gtttgactcctcaagaatgcatactttaatcttctagtacaacag
tttctttcaagatctcttttgtccattaatcagatagctatccct
gtttgtcttttgcaaatagccaatatgtcagtcgatctgtattct
gccttgcctatctttttttatctgttaatttcgtatggtgactca
tacaagttggtgcatctcctttaagttggggcttacccaaatatg
cagaacttgtttggagaactagggataaacgatcggttgcagtgg
aaggaacattcaatgatatttgcgatgcctaacaagccaggg
sgRNA for knock out mutagenesis of tobacco PDS gene
(SEQ ID NO: 36)
ggctgcatggaaagatgatgagttttagagctaggccaacatgag
gatcacccatgtctgcagggcctagcaagttaaaataaggctagt
ccgttatcaacttggccaacatgaggatcacccatgtctgcaggg
ccaagtggcaccgagtcggtgcttttttttttt
The mutated cas9 gene to generate dead
nuclease (dCas9),
Two mutations of Asp to Ala and His to Ala are
indicated in blue capital letters
(SEQ ID NO: 37)
atggacaagaagtacagcatcggcctgGCCatcggcacgaactcg
gtgggctgggcggtgatcacggacgagtacaaggtgccctccaag
aagttcaaggtgctgggcaacaccgaccgccactcgatcaagaag
aacctgatcggcgccctgctgttcgactccggcgagaccgccgag
gcgacgcgcctgaagcgcaccgcgcgtcgccgctacacgcgtcgc
aagaaccgcatctgctacctgcaggagatcttcagcaacgagatg
gccaaggtggacgactcgttcttccaccgcctggaggagtccttc
ctggtggaggaagacaagaagcacgagcgccaccccatcttcggc
aacatcgtggacgaggtggcctaccacgagaagtacccgacgatc
taccacctgcgcaagaagctggtggacagcaccgacaaggcggac
ctgcgcctgatctacctggccctggcgcacatgatcaagttccgc
ggccacttcctgatcgagggcgacctgaaccccgacaactcggac
gtggacaagctgttcatccagctggtgcagacctacaaccagctg
ttcgaggagaacccgatcaacgcctccggcgtggacgccaaggcg
atcctgagcgcgcgcctgtccaagagccgtcgcctggagaacctg
atcgcccagctgcccggcgagaagaagaacggcctgttcggcaac
ctgatcgcgctgtcgctgggcctgacgccgaacttcaagtccaac
ttcgacctggccgaggacgcgaagctgcagctgagcaaggacacc
tacgacgacgacctggacaacctgctggcccagatcggcgaccag
tacgcggacctgttcctggccgcgaagaacctgtcggacgccatc
ctgctgtccgacatcctgcgcgtgaacaccgagatcacgaaggcc
cccctgtcggcgtccatgatcaagcgctacgacgagcaccaccag
gacctgaccctgctgaaggcgctggtgcgccagcagctgccggag
aagtacaaggagatcttcttcgaccagagcaagaacggctacgcc
ggctacatcgacggggcgcgtcgcaagaggagttctacaagttca
tcaagcccatcctggagaagatggacggcacggaggagctgctgg
tgaagctgaaccgcgaggacctgctgcgcaagcagcgcaccttcg
acaacggcagcatcccccaccagatccacctgggcgagctgcacg
ccatcctgcgtcgccaagaggacttctacccgttcctgaaggaca
accgcgagaagatcgagaagatcctgacgttccgcatcccctact
acgtgggcccgctggcccgcggcaacagccgcttcgcgtggatga
cccgcaagtcggaggagaccatcacgccctggaacttcgaggaag
tggtggacaagggcgccagcgcgcagtcgttcatcgagcgcatga
ccaacttcgacaagaacctgcccaacgagaaggtgctgccgaagc
actccctgctgtacgagtacttcaccgtgtacaacgagctgacga
aggtgaagtacgtgaccgagggcatgcgcaagcccgccttcctga
gcggcgagcagaagaaggcgatcgtggacctgctgttcaagacca
accgcaaggtgacggtgaagcagctgaaagaggactacttcaaga
agatcgagtgcttcgacagcgtggagatctcgggcgtggaggacc
gcttcaacgccagcctgggcacctaccacgacctgctgaagatca
tcaaggacaaggacttcctggacaacgaggagaacgaggacatcc
tggaggacatcgtgctgaccctgacgctgttcgaggaccgcgaga
tgatcgaggagcgcctgaagacgtacgcccacctgttcgacgaca
aggtgatgaagcagctgaagcgtcgccgctacaccggctggggcc
gcctgagccgcaagctgatcaacggcatccgcgacaagcagtccg
gcaagaccatcctggacttcctgaagagcgacggcttcgcgaacc
gcaacttcatgcagctgatccacgacgactcgctgaccttcaaag
aggacatccagaaggcccaggtgtcgggccagggcgactccctgc
acgagcacatcgccaacctgggggctcccccgcgatcaagaaggg
catcctgcagaccgtgaaggtggtggacgagctggtgaaggtgat
gggccgccacaagccggagaacatcgtgatcgagatggcccgcga
gaaccagaccacgcagaagggccagaagaacagccgcgagcgcat
gaagcgcatcgaggaaggcatcaaggagctgggctcgcagatcct
gaaggagcaccccgtggagaacacccagctgcagaacgagaagct
ttacctgtactacctgcagaacggccgcgacatgtacgtggacca
ggagctggacatcaaccgcctgtccgactacgacgtggacGCCat
cgtgccccagagcttcctgaaggacgactcgatcgacaacaaggt
gctgacccgcagcgacaagaaccgcggcaagagcgacaacgtgcc
gtcggaggaagtggtgaagaagatgaagaactactggcgccagct
gctgaacgccaagctgatcacgcagcgcaagttcgacaacctgac
caaggccgagcgcggtggcctgtcggagctggacaaggcgggctt
catcaagcgccagctggtggagacccgccagatcacgaagcacgt
ggcgcagatcctggactcccgcatgaacacgaagtacgacgagaa
cgacaagctgatccgcgaggtgaaggtgatcaccctgaagtccaa
gctggtcagcgacttccgcaaggacttccagttctacaaggtgcg
cgagatcaacaactaccaccacgcccacgacgcgtacctgaacgc
cgtggtgggcaccgcgctgatcaagaagtaccccaagctggagag
cgagttcgtgtacggcgactacaaggtgtacgacgtgcgcaagat
gatcgccaagtcggagcaggagatcggcaaggccaccgcgaagta
cttcttctactccaacatcatgaacttcttcaagaccgagatcac
gctggccaacggcgagatccgcaagcgcccgctgatcgagaccaa
cggcgagacgggcgagatcgtgtgggacaagggccgcgacttcgc
gaccgtgcgcaaggtgctgagcatgccccaggtgaacatcgtgaa
gaagaccgaggtgcagacgggcggcttctccaaggagagcatcct
gccgaagcgcaactcggacaagctgatcgcccgcaagaaggactg
ggaccccaagaagtacggcggcttcgactccccgaccgtggccta
cagcgtgctggtggtggcgaaggtggagaagggcaagtccaagaa
gctgaagagcgtgaaggagctgctgggcatcaccatcatggagcg
cagctcgttcgagaagaaccccatcgacttcctggaggccaaggg
ctacaaagaggtgaagaaggacctgatcatcaagctgccgaagta
ctcgctgttcgagctggagaacggccgcaagcgcatgctggcctc
cgcgggcgagctgcagaagggcaacgagctggccctgcccagcaa
gtacgtgaacttcctgtacctggcgtcccactacgagaagctgaa
gggctcgccggaggacaacgagcagaagcagctgttcgtggagca
gcacaagcactacctggacgagatcatcgagcagatctcggagtt
ctccaagcgcgtgatcctggccgacgcgaacctggacaaggtgct
gagcgcctacaacaagcaccgcgacaagcccatccgcgagcaggc
ggagaacatcatccacctgttcaccctgacgaacctgggcgcccc
ggccgcgttcaagtacttcgacaccacgatcgaccgcaagcgcta
cacctccacgaaagaggtgctggacgcgaccctgatccaccagag
catcaccggcctgtacgagacgcgcatcgacctgagccagctggg
cggcgactcccgcgcggacccgaagaagaagcgcaaggtgagcgc
tggaggaggtggaagcggaggaggaggaagcggaggaggaggtag
c

1. A nucleic acid encoding a first fusion protein comprising a bacteriophage coat protein and a binding domain for an origin of replication.

2. A nucleic acid according to claim 1, wherein the fusion protein comprises a bacteriophage coat protein and a component of the replication initiation complex or replication complex.

3. A nucleic acid according to claim 1, wherein the fusion protein comprises a bacteriophage MS2 coat protein and a binding domain for an origin of replication.

4. A nucleic acid composition comprising a nucleic according to claim 1, further comprising a nucleic acid encoding an endonuclease.

5. A nucleic acid composition comprising a nucleic acid according to claim 1, further comprising a nucleic acid encoding an endonuclease wherein the endonuclease cleaves a target nucleic acid molecule in a sequence specific manner.

6. A nucleic acid composition comprising a nucleic acid according to claim 5, further comprising a nucleic acid encoding an endonuclease, wherein the endonuclease is an RNA-guided endonuclease.

7. A nucleic acid composition comprising a nucleic acid according to claim 6, further comprising a nucleic acid encoding an endonuclease, wherein the endonuclease is Cas9.

8. A nucleic acid composition comprising a nucleic acid according to claim 1, further comprising a nucleic acid encoding a second fusion protein comprising a 5′ to 3′ DNA exonuclease domain and an RNA binding domain.

9. A nucleic acid composition comprising a nucleic acid according to claim 1, further comprising a nucleic acid encoding a third fusion protein comprising a recombination inducing domain and an RNA binding domain.

10. A nucleic acid composition comprising a nucleic acid according to claim 1 further comprising a nucleic acid encoding a fourth fusion protein comprising a domain comprising an inhibitor of the mismatch repair pathway and an RNA binding domain.

11. The nucleic acid composition according to claim 10, wherein the inhibitor of the mismatch repair pathway is an inhibitor of MSH2 or MSH6.

12. The nucleic acid composition according to claim 10, wherein the inhibitor of the mismatch repair pathway comprises a dominant negative allele of MSH2 or MSH6.

13. A nucleic acid composition comprising a nucleic acid according to claim 1 further comprising a nucleic acid encoding a fifth fusion protein comprising a Holliday junction resolvase domain and an RNA binding domain.

14. A method of modifying the genome of a non-animal organism or cell comprising:
a. expressing in the cell a nucleic acid encoding a first fusion protein comprising a bacteriophage coat protein and a binding domain for an origin of replication or introducing the first fusion protein into the cell; and
b. expressing in the cell or introducing into the cell a donor nucleic acid molecule comprising an origin of replication.

15. A method according to claim 14, wherein the donor nucleic acid molecule comprises:
a. a donor nucleic acid sequence;
b. flanking nucleic acid sequences located 5′ and 3′ to the donor nucleic acid sequence;
c. an origin of replication 5′ to the 5′ flanking nucleotide sequence, and
d. a replication terminator 3′ to the 3′ flanking nucleotide sequence.

16. A method according to claim 14 for modifying a genome via homology-directed repair (HDR), comprising the step of introducing a double strand break into the genome in the presence of the donor nucleic acid molecule, wherein the donor nucleic acid molecule comprises a donor nucleic acid sequence as a template for modifying the genome, or partial or complete integration into the genome.

17. A method according to claim 14, further comprising the steps of:
expressing in the cell or introducing into the cell a sequence specific guide RNA to direct cleavage by an RNA-guided endonuclease to the specific sequence.

18. A method according to claim 14, wherein the binding domain for an origin of replication of the first fusion protein binds to the origin of replication of the donor nucleic acid.

19. A method according to claim 14, further comprising the steps of:
a. expressing in the cell a nucleic acid encoding an endonuclease, or introducing the endonuclease into the cell;
b. expressing in the cell a nucleic acid encoding a second fusion protein comprising a second fusion protein comprising a 5′ to 3′ DNA exonuclease domain and an RNA binding domain, or introducing the second fusion protein into the cell; and
c. expressing in the cell a nucleic acid encoding a fourth fusion protein comprising a domain comprising an inhibitor of the mismatch repair pathway and an RNA binding domain, or introducing the second fusion protein into the cell.
